SEQUENCE LISTING (38 sequences)

SEQ ID NO: 1 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 2 - protein, 401 aa, Homo sapiens
MNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKAQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 3 - protein, 117 aa, Homo sapiens
PQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGD

SEQ ID NO: 4 - protein, 139 aa, Homo sapiens
CPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSC

SEQ ID NO: 5 - protein, 174 aa, Homo sapiens
IAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKP

SEQ ID NO: 6 - protein, 173 aa, Homo sapiens
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQK

SEQ ID NO: 7 - DNA, 1047 bp, Artificial Sequence
  CDS (1)...(1047): pYhRANKb ORF region (Figure 6)
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaagcttctgcaggctagtggtggtggtggttctggtggtggtggttctggtggtggtggttctgctagcatgactggtggacagcaaatgggtcgggatctgtacgacgatgacgataaggtaccaattggcggtcgtactatcgctcctccatgtactagtgaaaaacactatgaacatttgggcagatgttgtaataagtgtgaacctggtaaatacatgtctagtaagtgtactaccactagtgacagtgtttgtttgccttgtggcccagatgaatatttggattcttggaacgaagaagataagtgtttgctacacaaagtttgtgacaccggtaaggctctagttgccgttgtcgctggcaacagtactaccccacgtagatgtgcatgcactgccggttatcactggtctcaagactgtgaatgttgtcgtagaaatactgaatgtgccccaggcttgggcgctcaacaccctttgcaattgaataaggatactgtctgtaaaccatgtttggccggttatttcagtgacgccttcagttctaccgacaaatgtcgtccatggactaattgtaccttcctaggtaaaagagtcgaacatcacggcactgaaaagagtgacgccgtttgttctagttctctgccggccagaaaaccacctaacgaaccacatgtttatttgcctggtctcgagtctagagggcccttcgaaggtaagcctatccctaaccctctcctcggtctcgattctacgcgtaccggtcatcatcaccatcaccat

SEQ ID NO: 8 - protein, 349 aa, Artificial Sequence
  polypeptide encoded by SEQ ID NO:7
MQLLRCFSIFSVIASVLAQELTTICEQIPSPTLESTPYSLSTTTILANGKAMQGVFEYYKSVTFVSNCGSHPSTTSKGSPINTQYVFKLLQASGGGGSGGGGSGGGGSASMTGGQQMGRDLYDDDDKVPIGGRTIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLESRGPFEGKPIPNPLLGLDSTRTGHHHHHH

SEQ ID NO: 9 - DNA, 1044 bp, Artificial Sequence
  CDS (1)...(1044): pYhRANKbE ORF region for production of soluble hRANK on the surface of yeast cells (Figure 7)
atgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaagcttctgcaggctagtggtggtggtggttctggtggtggtggttctggtggtggtggttctgctagcatgactggtggacagcaaatgggtcgggatctgtacgacgatgacgataaggtaccaattggcggtcgtactatcgctcctccatgtactagtgaaaaacactatgaacatttgggcagatgttgtaataagtgtgaacctggtaaatacatgtctagtaagtgtactaccactagtgacagtgtttgtttgccttgtggcccagatgaatatttggattcttggaacgaagaagataagtgtttgctacacaaagtttgtgacaccggtaaggctctagttgccgttgtcgctggcaacagtactaccccacgtagatgtgcatgcactgccggttatcactggtctcaagactgtgaatgttgtcgtagaaatactgaatgtgccccaggcttgggcgctcaacaccctttgcaattgaataaggatactgtctgtaaaccatgtttggccggttatttcagtgacgccttcagttctaccgacaaatgtcgtccatggactaattgtaccttcctaggtaaaagagtcgaacatcacggcactgaaaagagtgacgccgtttgttctagttctctgccggccagaaaaccacctaacgaaccacatgtttatttgcctggtctcgagtctagagggcccttcgaaggtgcgccggtgccgtatccggatccgctggaaccgcgtcgtaccggtcatcatcaccatcaccat

SEQ ID NO: 10 - protein, 348 aa, Artificial Sequence
  soluble hRANK polypeptide encoded by SEQ ID NO:9
MQLLRCFSIFSVIASVLAQELTTICEQIPSPTLESTPYSLSTTTILANGKAMQGVFEYYKSVTFVSNCGSHPSTTSKGSPINTQYVFKLLQASGGGGSGGGGSGGGGSASMTGGQQMGRDLYDDDDKVPIGGRTIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLESRGPFEGAPVPYPDPLEPRRTGHHHHHH

SEQ ID NO: 11 - DNA, 600 bp, Artificial Sequence
  CDS (1)...(600): RANKL sequence for production in baculovirus (Figure 8)
atgaataagttattgtgttgtgccttggtcttcttagacatctctatcaagtggactacccaagaaactagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagat

SEQ ID NO: 12 - protein, 200 aa, Artificial Sequence
  RANKL polypeptide encoded by SEQ ID NO:11
MNKLLCCALVFLDISIKWTTQETRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 13 - DNA, 798 bp, Artificial Sequence
  CDS (1)...(798): RANKL coding sequence for production in yeast (Figure 9)
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagat

SEQ ID NO: 14 - protein, 266 aa, Artificial Sequence [sequence omitted]

SEQ ID NO: 15 - DNA, 600 bp, Artificial Sequence
  CDS (2)...(559): RANKL coding sequence for production in E. coli (Figure 10)
catggactataaggatgacgacgataagcgtgcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctgggtgtt

SEQ ID NO: 16 - protein, 186 aa, Artificial Sequence
  RankL polypeptide encoded by SEQ ID NO:15
MDYKDDDDKRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 17 - DNA, 519 bp, Artificial Sequence
  CDS (1)...(519): coding sequence for OPG-TNFr like part codon optimised for yeast  (Figure 11)
gaaacttttccacctaagtatttgcattacgatgaagaaactagtcaccaattgctatgtgataagtgtccacctggaacctacctaaagcaacactgtactgccaagtggaagaccgtttgtgctccttgtcctgaccattactatactgattcttggcacacctctgatgaatgtttgtattgtagtccagtctgtaaagagctccaatatgttaaacaagaatgtaatagaacccacaatagagtctgtgaatgtaaagaaggcagatatttggaaattgagttctgtttgaaacaccgttcttgtccacctggttttggtgtcgttcaagctggcactcctgaacgtaacaccgtttgtaagcgttgtcctgacggcttcttttctaatgaaacctctagtaaggccccatgtagaaaacacaccaactgttctgtcttcggcttgctactgacccaaaaaggtaatgctacccacgataacatatgtagtggtaattctgaatctacccaaaaa

SEQ ID NO: 18 - protein, 173 aa, Artificial Sequence; identical to SEQ ID NO: 6
  OPG-TNFR like part encoded by SEQ ID NO:17

SEQ ID NO: 19 - DNA, 1275 bp, Artificial Sequence
  CDS (1)...(1275): ORF region of pcOPGbFc for production of OPG-Fc in mammalian cells (Figure 12)
atgaataagctgctgtgctgcgccctggtgttcctggacatcagcatcaagtggaccacacaggaaacttttccacctaagtatttgcattacgatgaagaaactagtcaccaattgctatgtgataagtgtccacctggaacctacctaaagcaacactgtactgccaagtggaagaccgtttgtgctccttgtcctgaccattactatactgattcttggcacacctctgatgaatgtttgtattgtagtccagtctgtaaagagctccaatatgttaaacaagaatgtaatagaacccacaatagagtctgtgaatgtaaagaaggcagatatttggaaattgagttctgtttgaaacaccgttcttgtccacctggttttggtgtcgttcaagctggcactcctgaacgtaacaccgtttgtaagcgttgtcctgacggcttcttttctaatgaaacctctagtaaggccccatgtagaaaacacaccaactgttctgtcttcggcttgctactgacccaaaaaggtaatgctacccacgataacatatgtagtggtaattctgaatctacccaaaaactcgagaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 20 - protein, 425 aa, Artificial Sequence
  OPG-Fc polypeptide encoded by SEQ ID NO:19
MNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKLEKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - DNA, 552 bp, Artificial Sequence
  CDS (1)...(552): cDNA encoding human RANK-TNFr like part, codon optimised for expression in yeast (Figure 13)
atcgctcctccatgtactagtgaaaaacactatgaacatttgggcagatgttgtaataagtgtgaacctggtaaatacatgtctagtaagtgtactaccactagtgacagtgtttgtttgccttgtggcccagatgaatatttggattcttggaacgaagaagataagtgtttgctacacaaagtttgtgacaccggtaaggctctagttgccgttgtcgctggcaacagtactaccccacgtagatgtgcatgcactgccggttatcactggtctcaagactgtgaatgttgtcgtagaaatactgaatgtgccccaggcttgggcgctcaacaccctttgcaattgaataaggatactgtctgtaaaccatgtttggccggttatttcagtgacgccttcagttctaccgacaaatgtcgtccatggactaattgtaccttcctaggtaaaagagtcgaacatcacggcactgaaaagagtgacgccgtttgttctagttctctgccggccagaaaaccacctaacgaaccacatgtttatttgcctggt

SEQ ID NO: 22 - protein, 184 aa, Artificial Sequence
  RANK-TNFr like part encoded by SEQ ID NO:21
IAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPG

SEQ ID NO: 23 - DNA, 1400 bp, Artificial Sequence
  CDS (17)...(1348): ORF region of pchRANKFc for production of hRANK-Fc in mammalian cells  (Figure 14)
gctggctagcgccgccatggcccccagagccagacggagacggcctctgtttgccctgctgctgctgtgcgccctgctggcccggctgcaggtggccctgcagatcgcccctccctgcacctccgagaagcactatgagcacctgggccggtgctgcaataagtgcgagcctggcaagtacatgagctccaagtgcaccacaacctccgactccgtgtgcctgccttgcggccccgatgagtatctggatagctggaacgaggaggataagtgcctgctgcacaaggtgtgcgacacaggcaaggccctggtggccgtggtggccggcaactccaccacacccagacggtgcgcctgcaccgccggctatcactggagccaggactgcgagtgctgcagacggaataccgagtgcgcccccggcctgggcgcccagcaccctctgcagctgaataaggataccgtgtgcaagccctgcctggccggctatttctccgacgccttctccagcacagacaagtgcagaccctggaccaattgcacattcctgggcaagcgggtggagcaccacggcaccgagaagtccgacgccgtgtgcagctccagcctgcccgcccggaagccccctaacgagccccacgtgtatctgcctggcctcgagaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttgatctagagggcccgtttaaacccgctgatcagcctcgactgtgccttcta

SEQ ID NO: SEQ ID N  O 24 - protein, 444 aa, Artificial Sequence
  hRANK-Fc protein encoded by SEQ ID NO:23
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLEKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - DNA, 1100 bp, Artificial Sequence
  CDS (35)...(1048): ORF region of pYhOPGb for production of soluble hOPG on surface of yeast cells  (Figure 15)
taagctaattctacttcatacattttcaattaagatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaagcttctgcaggctagtggtggtggtggttctggtggtggtggttctggtggtggtggttctgctagcatgactggtggacagcaaatgggtcgggatctgtacgacgatgacgataaggtaccaattggcggtcgtactgaaacttttccacctaagtatttgcattacgatgaagaaactagtcaccaattgctatgtgataagtgtccacctggaacctacctaaagcaacactgtactgccaagtggaagaccgtttgtgctccttgtcctgaccattactatactgattcttggcacacctctgatgaatgtttgtattgtagtccagtctgtaaagagctccaatatgttaaacaagaatgtaatagaacccacaatagagtctgtgaatgtaaagaaggcagatatttggaaattgagttctgtttgaaacaccgttcttgtccacctggttttggtgtcgttcaagctggcactcctgaacgtaacaccgtttgtaagcgttgtcctgacggcttcttttctaatgaaacctctagtaaggccccatgtagaaaacacaccaactgttctgtcttcggcttgctactgacccaaaaaggtaatgctacccacgataacatatgtagtggtaattctgaatctacccaaaaactcgagtctagagggcccttcgaaggtaagcctatccctaaccctctcctcggtctcgattctacgcgtaccggtcatcatcaccatcaccattgagtttaaacccgctgatctgataacaacagtgtagatgtaacaaaatcga

SEQ ID NO: 26 - protein, 338 aa, Artificial Sequence
  soluble hOPG polypeptide encoded by SEQ ID NO:25
MQLLRCFSIFSVIASVLAQELTTICEQIPSPTLESTPYSLSTTTILANGKAMQGVFEYYKSVTFVSNCGSHPSTTSKGSPINTQYVFKLLQASGGGGSGGGGSGGGGSASMTGGQQMGRDLYDDDDKVPIGGRTETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKLESRGPFEGKPIPNPLLGLDSTRTGHHHHHH

SEQ ID NO: 27 - protein, 6 aa, Artificial Sequence
  peptide tag
HHHHHH

SEQ ID NO: 28 - protein, 8 aa, Artificial Sequence
  peptide tag
MKHHHHHH

SEQ ID NO: 29 - protein, 10 aa, Artificial Sequence
  peptide tag
MKHHAHHQHH

SEQ ID NO: 30 - protein, 14 aa, Artificial Sequence
  peptide tag
MKHQHQHQHQHQHQ

SEQ ID NO: 31 - protein, 15 aa, Artificial Sequence
  peptide tag
MKHQHQHQHQHQHQQ

SEQ ID NO: 32 - protein, 10 aa, Artificial Sequence
  peptide tag
EQKLISEEDL

SEQ ID NO: 33 - protein, 8 aa, Artificial Sequence
  peptide tag
DYKDDDDK

SEQ ID NO: 34 - protein, 9 aa, Artificial Sequence
  peptide tag
YPYDVPDYA

SEQ ID NO: 35 - protein, 5 aa, Artificial Sequence
  linker peptide
GGGGS

SEQ ID NO: 36 - DNA, 51 bp, Artificial Sequence
  doped oligonucleotide
  misc_feature (20)...(22): NNN = 90% Tri-ATG / 10% Tri-CTG
  misc_feature (23)...(25): NNN = 90% Tri-TCT / 10% Tri-AAA
  misc_feature (26)...(28): NNN = 90% Tri-TCT / 10% Tri-CAG
  misc_feature (29)...(31): NNN = 90% Tri-AAA / 10% Tri-CAT
gtgtgaacctggtaaatacnnnnnnnnnnnntgtactaccactagtgacag

SEQ ID NO: 37 - DNA, 20 bp, Artificial Sequence
  primer
acgataaggtaccaatcgct

SEQ ID NO: 38 - DNA, 20 bp, Artificial Sequence
  primer
aatcgagaccgaggagaggg

(19) United States
(12) Patent Application Publication
(10) Pub. No.: US 2004/0132971 A1
(43) Pub. Date: Jul. 8, 2004

(54) Rank ligand-binding polypeptides

(76) Inventors: Jesper Mortensen Haaning, Birkeroed (DK); Torben Halkier, Solroed (DK)

Correspondence Address: MAXYGEN, INC., INTELLECTUAL PROPERTY DEPARTMENT, 515 GALVESTON DRIVE, RED WOOD CITY, CA, 94063, US

(21) Appl. No.: 10/467,243

(22) PCT Filed: Feb. 8, 2002
(86) PCT No.: PCT/DK02/00090

(30) Foreign Application Priority Data
Mar. 23, 2001 (DK) PA 2001 00498
Feb. 9, 2001 (DK) PA 2001 00214

(51) Int. Cl. C07K 14/705

(52) U.S. Cl. 530/350; 530/395

(57) ABSTRACT

The present invention relates to a polypeptide having an amino acid sequence that differs from and is at least 70% identical to the amino acid sequence of hRANK, and which has a binding affinity to RANKL that is at least as high as the binding affinity of hRANK to RANKL, as determined by the functional competition assay described herein.

### FIELD OF THE INVENTION

The present invention relates to novel polypeptides that are capable of binding to and antagonizing RANK ligand (RANKL), thereby reducing osteoclastogenesis and bone resorption, as well as nucleotide sequences encoding the antagonist polypeptides, methods for producing the antagonist polypeptides, and use of such antagonist polypeptides in therapy and for the manufacture of a medicament.

### BACKGROUND OF THE INVENTION

Osteoporosis is a systemic skeletal disease characterized by low bone mineral and micro-architectural deterioration of bone tissue, with a consequent increase in bone fragility and susceptibility to fracture. More than 20,000,000 people in the US, Europe and Japan are currently estimated to suffer from osteoporosis, primarily women, and this number is expected to increase significantly in the future along with the increased number of elderly persons. It is estimated that 50% of all women and 20% of all men will suffer an osteoporosis-related fracture at some point in their life, while 300,000 Americans per year will fracture a hip due to osteoporosis. Of those persons suffering hip fractures, 20% will not survive the first year, 50% will never walk independently again, and 25% will require institutional care. The cost of osteoporotic fractures per year in the US amounted to $13 billion in 1995. Thus, in terms of both patient suffering and economic costs, osteoporosis is a major and growing problem.

Osteoporotic patients can be categorized in four groups (for women aged 50+): 16% no risk, 50% low risk—osteopenia (often treated with hormone replacement therapy (HRT)), 14% osteoporotic (bone mineral density below threshold) with no fractures (treatment: HRT, calcitonin), and 16% osteoporotic with one or more fractures. For patients in the latter category, who require bone rebuilding therapy, there is at present no effective, commercially available treatment.

Another sometimes serious, but less widespread, bone disease is Paget's disease of bone, which is estimated to affect up to 1,000,000 people in the UK (http:www.paget.org.uk). Paget's disease of bone is a chronic skeletal disorder which results in enlarged or deformed bones in one or more regions of the skeleton. The deformed bone has an irregular structure and is consequently weaker, making it more prone to fracture than normal bone. Although only a relatively small proportion of patients with Paget's disease of bone suffer from serious symptoms, there is currently no effective treatment for this disease.

At the molecular level, there are three key protein players in osteoclastogenesis, i.e. the process by which osteoclast cells, which are active in bone resorption and hence in the regulation of bone degradation, develop and mature. These three proteins are:

RANKL (receptor activator of NF-kappaB ligand (Anderson, et al., (1997) Nature 390, 175-9)), also known as OPGL (osteoprotegerin ligand (Lacey et al., (1998) Cell 93, 165-76)), TRANCE (tumor necrosis factor-related activation-induced cytokine (Wong et al., (1997) J. Biol. Chem. 272, 25190-4)), and ODF (osteoclast differentiation factor (Yasuda, et al., (1998) PNAS 95, 3597-3602)),

OPG (osteoprotegerin (Simonet, et al., (1997) Cell 89, 309-19)), also known as OCIF (osteoclastogenesis inhibitory factor (Tsuda et al., (1997) Biochem. Biophys. Res. Commun. 234, 137-42)), and

RANK (receptor activator of NF-kappaB (Anderson, et al., (1997) Nature 390, 175-9)).

Briefly, the interactions of the three key proteins are as follows. RANKL is synthesized by osteoblasts/bone marrow stromal cells, where it is found on the cell surface. Binding of RANKL to its receptor, RANK, which is found on the surface of osteoclast precursor cells, activates a signaling pathway that leads to formation of mature osteoclasts and thus bone resorption. OPG acts as a soluble decoy receptor that binds RANKL, thereby inhibiting the binding of RANKL to RANK and thus inhibiting the formation of mature osteoclasts. For a review of the mechanisms involved in osteoclastogenesis and bone resorption, see Aubin et al., Medscape Women's Health 5(2), March/April 2000 (www.medscape.com).

For the treatment of osteoporosis, various forms of treatment are available, albeit none of the currently available treatments are fully safe and effective. These include hormone replacement therapy (HRT), estrogen replacement therapy (ERT), bisphosphonates such as alendronate sodium (Fosamax®) and risedronate sodium (Actonel®), selective estrogen receptor modulators (SERMs) such as raloxifene (Evista®), and calcitonin (Miacalcin®) (a naturally occurring hormone involved in calcium regulation and bone metabolism).

Various side effects have been reported for all of the current osteoporosis treatments and may include e.g. nausea, bloating, breast tenderness, high blood pressure and blood clots (ERT/HRT); abdominal or musculoskeletal pain, nausea, heartburn, or irritation of the esophagus (alendronate sodium); and allergic reaction, flushing of the face and hands, urinary frequency, nausea, and skin rash (calcitonin). Further, a possible relationship between estrogen use and breast cancer has been suggested. See e.g. www.nof.org/patientinfo/medications.htm regarding current osteoporosis medications.

Thus, although several different types of medications are available for the treatment of osteoporosis and other bone diseases, there is a large and unmet need for new medications that can provide an effective and long-lasting treatment of such diseases with a minimum of side effects.

### BRIEF DISCLOSURE OF THE INVENTION

The present invention addresses the problems discussed above and provides novel polypeptides and polypeptide conjugates suitable for use in the treatment of osteoporosis and other bone diseases.

The object of the present invention is to provide improved soluble osteodegeneration inhibitors by improving one or both of the following characteristics of RANK or OPG:

Firstly, the invention aims at improving the binding characteristics of the compounds RANK (residues [30-36]-[196-220] of Genbank acc. No. AF018253)) or OPG (residues [21-27]-[185-201] of Genbank acc. No. U94332) to RANKL, and secondly, the invention aims at improving the in vivo biological activity of the compounds by increasing the half-life, reducing the immunogenicity, increasing the physical size of the compounds, physically shielding the compounds from binding to other protein compounds in the human body, and/or producing the compounds as a dimer.

A first aspect of the invention relates to a polypeptide having an amino acid sequence that differs from and is least about 70% identical to the amino acid sequence of human RANK (hRANK), and which has a binding affinity to RANKL that is at least as high as the binding affinity of hRANK to RANKL, e.g. as determined by the functional competition assay described herein.

In one embodiment the polypeptide has an increased binding affinity to RANKL compared to the binding affinity of hRANK in the functional competition assay. In another embodiment the polypeptide has an amino acid sequence that is at least about 75% identical to the amino acid sequence of hRANK, e.g. at least about 80%, 85%, 90% or 95%. In a further embodiment the polypeptide has at least one non-polypeptide moiety bound to an attachment group of the polypeptide. In a further embodiment the non-polypeptide moiety is selected from the group consisting of polymer molecules, oligosaccharide moieties, lipophilic compounds and organic derivatizing agents. In a further embodiment the non-polypeptide moiety is a PEG molecule. In a further embodiment the polypeptide has an increased functional in vivo half-life and/or serum half-life compared to hRANK.

A second aspect of the invention relates to a polypeptide having an amino acid sequence that differs from and is least about 70% identical to the amino acid sequence of human OPG (hOPG), and which has a binding affinity to RANKL that is at least as high as the binding affinity of hOPG to RANKL, e.g. as determined by the functional competition assay described herein.

In one embodiment the polypeptide has an increased binding affinity to RANKL compared to the binding affinity of hOPG in the functional competition assay. In another embodiment the polypeptide has an amino acid sequence that is at least about 75% identical to the amino acid sequence of hOPG, e.g. at least about 80%, 85%, 90% or 95%. In a further embodiment the polypeptide has at least one non-polypeptide moiety bound to an attachment group of the polypeptide. In a further embodiment the non-polypeptide moiety is selected from the group consisting of polymer molecules, oligosaccharide moieties, lipophilic compounds and organic derivatizing agents. In a further embodiment the non-polypeptide moiety is a PEG molecule. In a further embodiment the polypeptide has an increased functional in vivo half-life and/or serum half-life compared to hOPG.

A third aspect of the invention relates to a polypeptide having an amino acid sequence that is at least 40% identical to the amino acid sequence of hRANK and at least 40% identical to the amino acid sequence of hOPG, and which has a binding affinity to RANKL at least as high as the binding affinity of hRANK and hOPG to RANKL, e.g. as determined by the functional competition assay described herein.

In one embodiment the polypeptide has an increased binding affinity to RANKL compared to the binding affinity of hRANK and hOPG in the functional competition assay. In another embodiment the polypeptide has an amino acid sequence that is at least about 45% identical to the amino acid sequence of hRANK and/or hOPG, e.g. at least about 50%, 55%, 60%, 65%, 70%, 75% or 80%. In a further embodiment the polypeptide has at least one non-polypeptide moiety bound to an attachment group of the polypeptide. In a further embodiment the non-polypeptide moiety is selected from the group consisting of polymer molecules, oligosaccharide moieties, lipophilic compounds and organic derivatizing agents. In a further embodiment the non-polypeptide moiety is a PEG molecule.

A fourth aspect of the invention relates to a chimeric polypeptide comprising a RANK backbone wherein at least one amino acid residue of the RANK backbone has been substituted with the corresponding amino acid residue from an OPG polypeptide as determined by a sequence alignment.

In one embodiment at least 2, preferably at least 3, e.g. at least 4, 5, 6, 7, 8, 9 or 10, such as up to about 15 or 20 amino acid residues of the RANK backbone have been substituted with the corresponding amino acid residues from the OPG polypeptide. In another embodiment at least one amino acid residue substitution is in the TNF receptor-like domain, preferably in a ligand binding domain. In a further embodiment the RANK backbone is hRANK. In a further embodiment the chimeric polypeptide has an improved binding affinity to RANKL compared to the binding affinity of hRANK to RANKL, eg. as determined by the functional competition assay described herein. In a further embodiment the chimeric polypeptide has at least one non-polypeptide moiety bound to an attachment group of the polypeptide.

A fifth further aspect of the invention relates to a chimeric polypeptide comprising an OPG backbone wherein at least one amino acid residue of the OPG backbone has been substituted with the corresponding amino acid residue from a RANK polypeptide as determined by a sequence alignment.

In one embodiment at least 2, preferably at least 3, e.g. at least 4, 5, 6, 7, 8, 9 or 10, such as up to about 15 or 20 amino acid residues of the OPG backbone have been substituted with the corresponding amino acid residues from the RANK polypeptide. In another embodiment at least one amino acid residue substitution is in the TNFR-like domain, preferably in a ligand binding domain. In a further embodiment the OPG backbone is hOPG. In a further embodiment the chimeric polypeptide has an improved binding affinity to RANKL compared to the binding affinity of hOPG to RANKL, eg. as determined by the functional competition assay described herein. In a further embodiment the chimeric polypeptide has at least one non-polypeptide moiety bound to an attachment group of the polypeptide.

A sixth aspect of the invention relates to a method for obtaining a nucleic acid encoding a recombinant polypeptide having RANKL binding activity, the method comprising:

(a) creating a library of recombinant polynucleotides encoding one or more recombinant RANK polypeptides; and

(b) screening the library to identify a recombinant polynucleotide encoding a recombinant polypeptide with a binding affinity to RANKL at least as high as the binding affinity of hRANK to RANKL.

In one embodiment the method comprises selecting at least one recombinant polynucleotide encoding a recombinant polypeptide with a binding affinity to RANKL higher than the binding affinity of hRANK to RANKL. In another embodiment the library is created by subjecting a plurality of parental polynucleotides to site-directed or random mutagenesis to produce at least one recombinant RANK polynucleotide encoding said improved recombinant polypeptide. In a further embodiment the library is created by shuffling a plurality of parental polynucleotides to produce at least one recombinant RANK polynucleotide encoding said improved recombinant polypeptide. In a further embodiment the parental polynucleotides are homologous. In a further embodiment the parental polynucleotides are shuffled in a plurality of cells selected from prokaryotes and eukaryotes, e.g. in eukaryotic cells selected from bacteria, yeast, fungi and mammalian cells. In a further embodiment the method further comprises:

(c) recombining at least one distinct or improved recombinant polynucleotide with a further polynucleotide encoding a polypeptide with RANKL binding affinity, which further polynucleotide is identical to or different from one or more of said plurality of parental polynucleotides, to produce a library of recombinant polynucleotides;

(d) screening said library to identify at least one further distinct or improved recombinant polynucleotide encoding a RANKL binding polypeptide that exhibits a further improvement or distinct property compared to a polypeptide encoded by said plurality of parental polynucleotides; and, optionally,

(e) repeating (c) and (d) until said resulting further distinct or improved recombinant polynucleotide shows an additionally distinct or improved property. In a further embodiment the recombinant polynucleotides are present in one or more cells selected from bacterial, yeast, fungal and mammalian cells, and said method comprises:

pooling multiple separate polynucleotides;

screening said resulting pooled polynucleotides to identify an improved recombinant polynucleotide encoding a polypeptide that exhibits an improved binding affinity to RANKL compared to a polypeptide encoded by a non-recombinant activity polynucleotide; and

cloning said improved recombinant nucleic acid. In a further embodiment the method further comprises transducing said improved polynucleotide into a member selected from a prokaryote and a eukaryote. In a further embodiment the shuffling of a plurality of parental polynucleotides comprises at least one shuffling technique selected from family gene shuffling, individual gene shuffling and in silico shuffling.

A seventh aspect of the invention relates to a library of recombinant polynucleotides encoding at least one polypeptide with binding affinity to RANKL, wherein said library is made by the method for obtaining a nucleic acid encoding a recombinant polypeptide having RANKL binding activity.

In one embodiment of the library the polypeptides encoded by said recombinant polynucleotides are displayed on the surface of phage, bacteria cells, yeast cells or mammalian cells. An eighth aspect of the invention relates to a nucleic acid encoding a polypeptide with binding affinity to RANKL, wherein said nucleic acid is prepared by the method for obtaining a nucleic acid encoding a recombinant polypeptide having RANKL binding activity.

A ninth aspect of the invention relates to a nucleic acid shuffling mixture, comprising: at least three homologous DNAs, each of which is derived from a polynucleotide encoding a polypeptide selected from a parent RANK polypeptide, a polypeptide fragment having RANKL binding affinity, and combinations thereof.

In one embodiment the at least three homologous DNAs are present in cell culture or in vitro.

A further aspect of the invention relates to a polypeptide having RANKL binding affinity encoded by a nucleic acid produced by the method for obtaining a nucleic acid encoding a recombinant polypeptide having RANKL binding activity.

A further aspect of the invention relates to a method for obtaining a nucleic acid encoding a recombinant polypeptide having RANKL binding activity, the method comprising:

(a) creating a library of recombinant polynucleotides encoding one or more recombinant OPG polypeptides; and

(b) screening the library to identify a recombinant polynucleotide encoding a recombinant polypeptide with a binding affinity to RANKL at least as high as the binding affinity of hOPG to RANKL.

In one embodiment the method comprises selecting at least one recombinant polynucleotide encoding a recombinant polypeptide with a binding affinity to RANKL higher than the binding affinity of hOPG to RANKL. In a further embodiment the library is created by subjecting a plurality of parental polynucleotides to site-directed or random mutagenesis to produce at least one recombinant OPG polynucleotide encoding said improved recombinant polypeptide. In a further embodiment the library is created by shuffling a plurality of parental polynucleotides to produce at least one recombinant OPG polynucleotide encoding said improved recombinant polypeptide. In a further embodiment the parental polynucleotides are homologous. In a further embodiment the parental polynucleotides are shuffled in a plurality of cells selected from prokaryotes and eukaryotes, e.g. in eukaryotic cells selected from bacteria, yeast, fungi and mammalian cells. In a further embodiment the method further comprises:

(c) recombining at least one distinct or improved recombinant polynucleotide with a further polynucleotide encoding a polypeptide with RANKL binding affinity, which further polynucleotide is identical to or different from one or more of said plurality of parental polynucleotides, to produce a library of recombinant polynucleotides;

(d) screening said library to identify at least one further distinct or improved recombinant polynucleotide encoding a RANKL binding polypeptide that exhibits a further improvement or distinct property compared to a polypeptide encoded by said plurality of parental polynucleotides; and, optionally,

(e) repeating (c) and (d) until said resulting further distinct or improved recombinant polynucleotide shows an additionally distinct or improved property. In a further embodiment the recombinant polynucleotides are present in one or more cells selected from bacterial, yeast, fungal and mammalian cells, and said method comprises:

pooling multiple separate polynucleotides;

screening said resulting pooled polynucleotides to identify an improved recombinant polynucleotide encoding a polypeptide that exhibits an improved binding affinity to RANKL compared to a polypeptide encoded by a non-recombinant activity polynucleotide; and

cloning said improved recombinant nucleic acid. In a further embodiment the method further comprises transducing said improved polynucleotide into a member selected from a prokaryote and a eukaryote. In a further embodiment the shuffling of a plurality of parental polynucleotides comprises at least one shuffling technique selected from family gene shuffling, individual gene shuffling and in silico shuffling.

A further aspect of the invention relates to a library of recombinant polynucleotides encoding at least one polypeptide with binding affinity to RANKL, wherein said library is made by the method for obtaining a nucleic acid encoding a recombinant polypeptide having RANKL binding activity.

In one embodiment the polypeptides encoded by said recombinant polynucleotides are displayed on the surface of phage, bacteria cells, yeast cells or mammalian cells.

A further aspect of the invention relates to a nucleic acid encoding a polypeptide with binding affinity to RANKL, wherein said nucleic acid is prepared by the method for obtaining a nucleic acid encoding a recombinant polypeptide having RANKL binding activity.

A further aspect of the invention relates to a nucleic acid shuffling mixture, comprising: at least three homologous DNAs, each of which is derived from a polynucleotide encoding a polypeptide selected from a parent OPG polypeptide, a polypeptide fragment having RANKL binding affinity, and combinations thereof.

In one embodiment of the nucleic acid shuffling mixture the at least three homologous DNAs are present in cell culture or in vitro.

A further aspect of the invention relates to a polypeptide having RANKL binding affinity encoded by a nucleic acid produced by the method for obtaining a nucleic acid encoding a recombinant polypeptide having RANKL binding activity.

A further aspect of the invention relates to a polypeptide conjugate exhibiting RANKL-binding activity, comprising a RANK polypeptide that differs from wild-type human RANK in that at least one amino acid residue acid residue comprising an attachment group for a non-polypeptide moiety has been introduced or removed, and having at least one non-polypeptide moiety bound to an attachment group of the polypeptide.

In one embodiment the RANK polypeptide is a RANK variant of the first aspect or fourth aspect or encoded by a nucleic acid produced by the method of the sixth aspect.

A further aspect of the invention relates to a polypeptide conjugate exhibiting RANKL-binding activity, comprising an OPG polypeptide that differs from wild-type human OPG in that at least one amino acid residue acid residue comprising an attachment group for a non-polypeptide moiety has been introduced or removed, and having at least one non-polypeptide moiety bound to an attachment group of the polypeptide.

A further aspect of the invention relates to a polypeptide conjugate exhibiting RANKL-binding activity, comprising an OPG polypeptide that differs from wild-type human OPG in that at least one amino acid residue acid residue comprising an attachment group for a non-polypeptide moiety has been introduced or removed, and having at least one non-polypeptide moiety bound to an attachment group of the polypeptide.

In one embodiment the OPG polypeptide is an OPG variant of the second aspect or fifth aspect or encoded by a nucleic acid produced by the method of the sixth aspect.

A further aspect of the invention relates to an oligomeric fusion protein comprising at least two RANK monomers, at least two OPG monomers, or at least one RANK monomer and at least one OPG monomer, wherein at least one monomer of the fusion protein is a RANK and/or OPG variant of the above aspects or encoded by a nucleic acid produced by the method of the above aspects.

In one embodiment of the fusion protein the monomers are joined by a peptide bond or a peptide linker, or by a PEG molecule. In a further embodiment of the fusion protein comprising at least one RANKL-binding monomeric fusion protein, the monomeric fusion protein is produced as a protein fused in frame with an immunoglobulin Fc polypeptide or a GCN4 leucine zipper. A further aspect of the invention relates to a composition comprising a polypeptide according to any of the above polypeptide aspects or encoded by a nucleic acid produced by the method of any of the above method aspects, and at least one pharmaceutically acceptable carrier or excipient.

A further aspect of the invention relates to use of a polypeptide according to any of the above polypeptide aspects or encoded by a nucleic acid produced by the method of any of the above method aspects, or a composition of the above composition aspect, as a pharmaceutical.

A further aspect of the invention relates to use of a polypeptide according to any of the above polypeptide aspects or encoded by a nucleic acid produced by the method of any of the above method aspects, or a composition of the above composition aspect, for the preparation of a medicament for the prevention or treatment of osteoporosis or other bone diseases or other diseases associated with binding of RANKL to the RANK receptor.

A further aspect of the invention relates to a method for preventing or treating osteoporosis or other bone diseases or other diseases associated with binding of RANKL to the RANK receptor, the method comprising administering to a patient in need thereof an effective amount of a polypeptide according to any of the above polypeptide aspects or encoded by a nucleic acid produced by the method of any of the above method aspects, or a composition of the above composition aspect.

A further aspect of the invention relates to an expression vector comprising a nucleic acid produced by the method of any of the above method aspects.

A further aspect of the invention relates to a host cell comprising an expression vector according to the above expression vector aspect.

A further aspect of the invention relates to a method for producing a polypeptide having binding affinity to RANKL, comprising culturing a host cell according to the above host cell aspect under conditions conducive for expression of the polypeptide, and recovering the polypeptide.

In one embodiment of the method a) the polypeptide comprises at least one N- or O-glycosylation site and the host cell is a eukaryotic host cell capable of in vivo glycosylation, and/or b) the polypeptide is subjected to conjugation to a non-polypeptide moiety in vitro.

A further aspect of the invention relates to a chimeric polypeptide comprising a RANK backbone wherein at least one amino acid residue of the RANK backbone has been substituted with the corresponding amino acid residue from an OPG polypeptide as determined by a sequence alignment, comprising all or part of at least one TNF receptor-like domain of OPG as defined in FIG. 4B.

In one embodiment the part comprises at least one ligand binding subsequence of OPG comprising at least three amino acid residues as defined in FIG. 4B.

A further aspect of the invention relates to a chimeric polypeptide comprising an OPG backbone wherein at least one amino acid residue of the OPG backbone has been substituted with the corresponding amino acid residue from a RANK polypeptide as determined by a sequence alignment, comprising all or part of at least one TNF receptor-like domain of RANK as defined in FIG. 4B.

In one embodiment the part comprises at least one ligand binding subsequence of RANK comprising at least three amino acid residues as defined in FIG. 4B.

A further aspect of the invention relates to a method for obtaining a nucleic acid encoding a recombinant polypeptide having a desired RANKL binding activity, the method comprising:

(a) providing a polynucleotide encoding a recombinant chimeric polypeptide comprising at least one ligand binding sequence from an OPG domain and at least one ligand binding sequence from a RANK domain;

(b) subjecting said polynucleotide to mutagenesis to create a library of recombinant polynucleotides encoding one or more recombinant chimeric polypeptides; and

(c) screening the library to identify a recombinant polynucleotide encoding a recombinant polypeptide with a desired binding affinity to RANKL.

In one embodiment the recombinant chimeric polypeptide in (a) comprises at least one OPG domain and at least one RANK domain. In a further embodiment the mutagenesis is performed using at least one of site-directed mutagenesis, random mutagenesis and shuffling. Additional aspects and preferred embodiments of the invention will be apparent from the detailed disclosure below, including the claims, figures and sequences.

### BRIEF DESCRIPTION OF FIGURES

FIG. 1 shows Wild type full length RANK amino acid sequence. The cysteine rich RANKL binding domain is underlined.

FIG. 2 shows Wild type full length OPG amino acid sequence. The cysteine rich RANKL binding domain is underlined.

FIG. 3 shows a sequence alignment of Death Receptor 5 and TNF receptor gp55. The underlined amino acid residues are defined as being directly involved in ligand binding.

FIG. 4A shows a sequence alignment of OPG, RANK, Death Receptor 5, and TNF receptor gp55. The underlined amino acid residue stretches are defined as being directly involved in ligand binding.

FIG. 4B shows a sequence alignment of the TNFR-like domains of OPG and RANK. The amino acid positions are numbered above the alignment. Hashmarks indicate predicted domain boundaries. The predicted ligand binding residues are underlined.

FIG. 5 shows FACS analysis of OPG-displaying yeast.

FIG. 6 shows sequence 1: PYhRANKb.

FIG. 7 shows sequence 2: pYhRANKbE—For production of soluble hRANK on the surface of yeast cells (open reading frame only).

FIG. 8 shows sequence 3: RANKL for production in baculovirus (only the ORF is shown).

FIG. 9 shows sequence 4: RANKL for production in yeast (cerevisiae or Pichia pastoris).

FIG. 10 shows sequence 5: RANKL for production in E. coli.

FIG. 11 shows sequence 6: cDNA sequence encoding human OPG—TNFr like part. Codon optimised.

FIG. 12 shows sequence 7: pcOPGbFc—for production of OPG-Fc from mammalian cells (ORF only).

FIG. 13 shows sequence 8: cDNA sequence encoding human RANK-TNFr like part.

FIG. 14 shows sequence 9: pchRANKFc—For production of hRANK-Fc fusion protein from mammalian cells.

FIG. 15 shows sequence 10: pYhOPGb—For production of “soluble” hOPG on the surface of yeast cells.

### DETAILED DESCRIPTION OF THE INVENTION

The inventors have provided OPG variants with improved K_{d} in relation to wild-type hOPG. In particular, they have discovered that the amino acid residues T71, K108, R111, and T154 located in the four cysteine-rich TNF receptor-like domains of human OPG are involved in the binding to RANKL. In OPG, these four domains are found within residues 22-194 of hOPG. The substitution of one or more of the amino acid residues selected from T71, K108, R111, and T154 of hOPG with a different amino acid resulted in several instances in OPG variants with improved K_{d} in relation to wild-type hOPG.

Accordingly, in a further aspect the invention relates to a polypeptide having an amino acid sequence that is least about 70% identical to the amino acid sequence of hOPG(22-194) and wherein one or more of the amino acid residues selected from T71, K108, R111, and T154 have been substituted with a different amino acid residue.

In a still further aspect the invention relates to a polypeptide comprising the amino acid sequence hOPG(22-194) wherein one or more of the amino acid residues selected from T71, K108, R111, and T154 have been substituted with a different amino acid residue.

Thus, the polypeptide may comprise the full length of hOPG (shown in FIG. 2) or fragments, or variants thereof, wherein one or more of the amino acid residues selected from T71, K108, R111, and T154 have been substituted with a different amino acid residue.

hOPG(22-194) is intended to indicate the amino acid residues of position 22 to 194 of human OPG, thus having the sequence: etfppkylhydeetshqllcdkcppgtylkqhctakwktvcapcpdhy tdswhtsdeclycspvckelqyvkqecnrthnrvceckegryleiefclkhrscppgfgvvqagtperntvckrcpdgffsnetsska pcrkhtncsvfgllltqkgnathdnicsgnsestqk.

In one embodiment T71 has been substituted with A. In another embodiment K108 has been substituted with N. In a further embodiment R111 has been substituted with W. In a further embodiment T154 has been substituted with L. In a further embodiment T71 has been substituted with A and K108 has been substituted with N. In a further embodiment K108 has been substituted with N and R111 has been substituted with W.

In a still further embodiment the polypeptide comprises T71A,K108N-hOPG(22-194).

In a still further embodiment the polypeptide comprises R111W-hOPG(22-194).

In a still further embodiment the polypeptide comprises K108M,R111W-hOPG(22-194).

In a still further embodiment the polypeptide comprises T154L-hOPG(22-194).

In a still further embodiment the polypeptide is selected from the group comprising T71A,K108N-hOPG(22-194), R111W-hOPG(22-194), K108M,R111W-hOPG(22-194), and T154L-hOPG(22-194).

Definitions

In the context of the present application and invention the following definitions apply:

The term “RANK and/or OPG variant” (or “RANK- and/or OPG-related polypeptide”) is intended to indicate a polypeptide variant as described herein which is a variant of RANK or OPG, or which is a shuffled variant based on shuffling of both RANK and OPG or another chimeric variant based on RANK and OPG, as described in detail below.

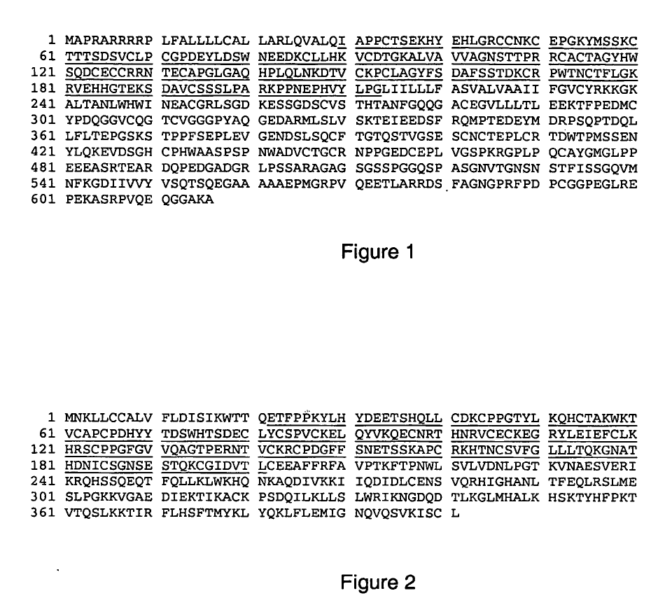

The RANK and/or OPG polypeptides used as parent polypeptides may be human RANK (hRANK) or human OPG (hOPG), and/or they may be homologous polypeptides. The amino acid sequence of hRANK is published in Anderson, et al., (1997) Nature 390, 175-9 and is shown in FIG. 1. The amino acid sequence of hOPG is published in Simonet, et al., (1997) Cell 89, 309-19 and is shown in FIG. 2.

As used herein, sequences that are judged to be derived by descent from a common ancestor comprise a “homologous gene family”, and the mutagenesis techniques described herein such as DNA shuffling can be used to accelerate the evolution of these gene families. Furthermore, many distinct protein sequences are consistent with similar protein folds, and such families of sequences can be said to comprise “structurally homologous” gene families. The TNF-receptor superfamily of structures, which include the ligand binding domains of both RANK and OPG, are such a family. The term “homologous” is intended to include homologous gene families, including homologous genes of related species, and structurally homologous gene families.

The term “conjugate” (or interchangeably “conjugated polypeptide”) is intended to indicate a heterogeneous (in the sense of composite or chimeric) molecule formed by the covalent attachment of a RANK and/or OPG variant to one or more “non-polypeptide moieties”. The term “covalent attachment” means that the polypeptide and the non-polypeptide moiety are either directly covalently joined to one another, or else are indirectly covalently joined to one another through an intervening moiety or moieties, such as a bridge, spacer, or linkage moiety or moieties using an attachment group present in the polypeptide. Preferably, the conjugate is soluble at relevant concentrations and conditions, i.e. soluble in physiological fluids such as blood. Examples of conjugated polypeptides of the invention include glycosylated and/or PEGylated polypeptides. The term “non-conjugated polypeptide” may be used about the polypeptide part of the conjugate.

The term “non-polypeptide moiety”, which may also be termed a “macromolecular moiety” or “macromolecule”, is intended to indicate a molecule that is capable of conjugating to an attachment group of the polypeptide of the invention. Preferred examples of such a molecule include polymer molecules, oligosaccharide moieties, lipophilic compounds, and organic derivatizing agents. When used in the context of a conjugate of the invention it will be understood that the non-polypeptide moiety is linked to the polypeptide part of the conjugate through an attachment group of the polypeptide.

The term “polymer molecule” is defined as a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is intended to cover carbohydrate molecules attached by in vitro glycosylation, i.e. a synthetic glycosylation performed in vitro normally involving covalently linking a carbohydrate molecule to an attachment group of the polypeptide, optionally using a cross-linking agent. Carbohydrate molecules attached by in vivo glycosylation, such as N- or O-glycosylation (as further described below)) are referred to herein as “an oligosaccharide moiety”. Except where the number of non-polypeptide moieties, such as polymer molecule(s) or oligosaccharide moieties in the conjugate is expressly indicated, every reference to “a non-polypeptide moiety” contained in a conjugate or otherwise used in the present invention shall be a reference to one or more non-polypeptide moieties, such as polymer molecules or oligosaccharide moieties, in the conjugate.

The term “attachment group” is intended to indicate an amino acid residue group of the polypeptide capable of coupling to the relevant non-polypeptide moiety. For instance, for polymer conjugation to PEG, a frequently used attachment group is the ε-amino group of lysine or the N-terminal amino group. Other polymer attachment groups include a free carboxylic acid group (e.g. that of the C-terminal amino acid residue or of an aspartic acid or glutamic acid residue), suitably activated carbonyl groups, oxidized carbohydrate moieties and mercapto groups. Useful attachment groups and their matching non-peptide moieties are apparent from the table below.

| | | | Conjugation | |
|---|---|---|---|---|
| Attachment | | Examples of non- | method/- | |
| group | Amino acid | peptide moiety | Activated PEG | Reference |
| —NH₂ | N-terminal, | Polymer, e.g. PEG, | mPEG-SPA | Shearwater Inc. |
| | Lys | with amide or imine | Tresylated mPEG | Delgado et al, critical |
| | | group | | reviews in Therapeutic |
| | | | | Drug Carrier Systems |
| | | | | 9(3, 4): 249-304 |
| | | | | (1992) |
| —COOH | C-term, | Polymer, e.g. PEG, | mPEG-Hz | Shearwater Inc. |
| | Asp, Glu | with ester or amide | | |
| | | group | | |
| | | Oligosaccharide | In vitro coupling | |
| | | moiety | | |
| —SH | Cys | Polymer, e.g. PEG, | PEG- | Shearwater Inc. |
| | | with disulfide, | vinylsulphone | Delgado et al, critical |
| | | maleimide or vinyl | PEG-maleimide | reviews in Therapeutic |
| | | sulfone group | | Drug Carrier Systems |
| | | Oligosaccharide | In vitro coupling | 9(3, 4): 249-304 |
| | | moiety | | (1992) |
| —OH | Ser, Thr, | Oligosaccharide | In vivo O-linked | |
| | OH—, Lys | moiety | glycosylation | |
| | | PEG with ester, | | |
| | | ether, carbamate, | | |
| | | carbonate | | |
| —CONH₂ | Asn as part | Oligosaccharide | In vivo N- | |
| | of an N- | moiety | glycosylation | |
| | glycosylation | Polymer, e.g. PEG | | |
| | site | | | |
| Aromatic | Phe, Tyr, | Oligosaccharide | In vitro coupling | |
| residue | Trp | moiety | | |
| —CONH₂ | Gln | Oligosaccharide | In vitro coupling | Yan and Wold, Bio- |
| | | moiety | | chemistry, 1984, Jul |
| | | | | 31; 23(16): 3759-65 |
| Aldehyde | Oxidized | Polymer, e.g. PEG, | PEGylation | Andresz et al., 1978, |
| Ketone | oligosaccharide | PEG-hydrazide | | Makromol. Chem. |
| | | | | 179: 301, WO |
| | | | | 92/16555, WO |
| | | | | 00/23114 |
| Guanidino | Arg | Oligosaccharide | In vitro coupling | Lundblad and Noyes, |
| | | moiety | | Chimical Reagents |
| | | | | for Protein Modification, |
| | | | | CRC Press Inc., |
| | | | | Florida, USA |
| Imidazole | His | Oligosaccharide | In vitro coupling | As for guanidine |
| ring | | moiety | | |

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue which may or may not be identical to X′ and which preferably is different from proline, N is asparagine, and S/T/C is either serine, threonine or cysteine, preferably serine or threonine, and most preferably threonine). Although the asparagine residue of the N-glycosylation site is where the oligosaccharide moiety is attached during glycosylation, such attachment cannot be achieved unless the other amino acid residues of the N-glycosylation site are present. Accordingly, when the non-peptide moiety is an oligosaccharide moiety and the conjugation is to be achieved by N-glycosylation, the term “amino acid residue comprising an attachment group for the non-peptide moiety” as used in connection with alterations of the amino acid sequence of the polypeptide of interest is to be understood as meaning that one or more amino acid residues constituting an N-glycosylation site are to be altered in such a manner that either a functional N-glycosylation site is introduced into the amino acid sequence or removed from said sequence.

In the present application, amino acid names and atom names (e.g. CA, CB, CD, CG, SG, NZ, N, O, C, etc.) are used as defined by the Protein DataBank (PDB) (www.pdb.org) which are based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). CA is sometimes referred to as Cα, CB as Cβ. The term “amino acid residue” is intended to indicate any amino acid residue, and in particular an amino acid residue selected from among the 20 naturally occurring amino acid residues, i.e. contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terminology used for identifying amino acid positions/substitutions is illustrated as follows: C133 indicates position 133 occupied by a cysteine residue in a given amino acid sequence. C133S indicates that the cysteine residue of position 133 has been replaced with a serine. Multiple substitutions are indicated with a “+”, e.g. K38R+R181K means an amino acid sequence which comprises a substitution of an lysine residue in position 38 with an arginine and a substitution of the arginine residue in position 181 with a lysine residue. An indication such as T/S as used about a given substitution herein, e.g. A103T/S, means either a T or an S residue.

The term “nucleotide sequence” or “polynucleotide sequence” is intended to indicate a polymer of two or more nucleotides or a character string representing a nucleotide sequence. The nucleotide sequence may be of genomic, cDNA, RNA, semisynthetic, synthetic origin, or any combination thereof. Either the given nucleic acid or the complementary nucleic acid can be determined from any specified polynucleotide sequence.

Similarly, an “amino acid sequence” is a polymer of amino acids (a protein, polypeptide, etc.) or a character string representing an amino acid polymer, depending on context.

A nucleic acid, protein or other component is “isolated” when it is partially or completely separated from components with which it is normally associated (other proteins, nucleic acids, cells, synthetic reagents, etc.). A nucleic acid or polypeptide is “recombinant” when it is artificial or engineered, or derived from an artificial or engineered protein or nucleic acid.

A “subsequence” or “fragment” is any portion of an entire sequence, up to and including the complete sequence.

A vector is a composition for facilitating cell transduction by a selected nucleic acid, or expression of the nucleic acid in the cell. Vectors include, e.g., plasmids, cosmids, viruses, YACs, bacteria, poly-lysine, etc.

“Substantially an entire length of a polynucleotide or amino acid sequence” refers to at least 70%, generally at least 80%, or typically 90% or more of a sequence.

The term “polymerase chain reaction” or “PCR” generally refers to a method for amplification of a desired nucleotide sequence in vitro as described, for example, in U.S. Pat. No. 4,683,195. In general, the PCR method involves repeated cycles of primer extension synthesis, using oligonucleotide primers capable of hybridising preferentially to a template nucleic acid.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The terms “homology” and “identity” as used in connection with amino acid sequences are used in their conventional meanings. Amino acid sequence homology/identity is conveniently determined from aligned sequences, using e.g. the ClustalW program or from the PFAM families database version 4.0 (http://pfam.wustl.edu/)(Nucleic Acids Res. Jan. 1, 1999; 27(1):260-2) by use of GENEDOC version 2.5 (Nicholas, K. B., Nicholas H. B. Jr., and Deerfield, D. W. II. 1997 GeneDoc: Analysis and Visualization of Genetic Variation, EMB-NEW.NEWS 4:14; Nicholas, K. B. and Nicholas H. B. Jr. 1997 GeneDoc: Analysis and Visualization of Genetic Variation).

The term “introduce” refers to introduction of an amino acid residue comprising an attachment group for a non-polypeptide moiety, either by substitution of an existing amino acid residue or by insertion of an additional amino acid residue. The term “remove” refers to removal of an amino acid residue comprising an attachment group for a non-polypeptide moiety, either by substitution of the amino acid residue to be removed by another amino acid residue or by deletion (without substitution) of the amino acid residue to be removed.

When substitutions are performed in relation to a parent RANK or OPG polypeptide, they may be “conservative substitutions”, in other words substitutions performed within groups of amino acids with similar characteristics, e.g. small amino acids, acidic amino acids, polar amino acids, basic amino acids, hydrophobic amino acids and aromatic amino acids. Conservative substitutions may in particular be chosen from among the conservative substitution groups listed in the table below.

Conservative Substitution Groups:

| | | | | |
|---|---|---|---|---|
| 1 | Alanine (A) | Glycine (G) | Serine (S) | Threonine |
| | | | | (T) |
| 2 | Aspartic acid (D) | Glutamic acid (E) | | |
| 3 | Asparagine (N) | Glutamine (Q) | | |
| 4 | Arginine (R) | Histidine (H) | Lysine (K) | |
| 5 | Isoleucine (I) | Leucine (L) | Methionine (M) | Valine |
| | | | | (V) |
| 6 | Phenylalanine (F) | Tyrosine (Y) | Tryptophan (W) | |

The term “functional in vivo half-life” is used in its normal meaning, i.e. the time at which 50% of the biological activity of the polypeptide or conjugate is still present in the body/target organ, or the time at which the activity of the polypeptide or conjugate is 50% of the initial value. As an alternative to determining functional in vivo half-life, “serum half-life” may be determined, i.e. the time in which 50% of the polypeptide or conjugate molecules circulate in the plasma or bloodstream prior to being cleared. Alternative terms to serum half-life include “plasma half-life”, “circulating half-life”, “serum clearance”, “plasma clearance” and “clearance half-life”. The polypeptide or conjugate is cleared by the action of one or more of the reticuloendothelial systems (RES), kidney, spleen or liver, by receptor-mediated degradation, or by specific or non-specific proteolysis, in particular by the action of receptor-mediated clearance and renal clearance. Normally, clearance depends on size (relative to the cutoff for glomerular filtration), charge, attached carbohydrate chains, and the presence of cellular receptors for the protein. The functionality to be retained in the present context is normally binding to RANKL. The functional in vivo half-life or the serum half-life may be determined by methods known in the art.

The term “increased” as used about the functional in vivo half-life or serum half-life is used to indicate that the relevant half-life of the conjugate or polypeptide is statistically significantly increased relative to that of a reference molecule, such as a non-conjugated hRANK or hOPG, as determined under comparable conditions. For instance, the relevant half-life may increased by at least about 25%, such as by at least about 50%, e.g. by at least about 100%, 200%, 500% or 1000%.

Introduction and Removal of Attachment Groups

In a preferred embodiment, the RANK and/or OPG variants of the invention are conjugated to at least one non-polypeptide moiety. By removing and/or introducing one or more amino acid residues comprising an attachment group for the non-polypeptide moiety, it is possible to specifically adapt the polypeptide so as to make the molecule more susceptible to conjugation to the non-polypeptide moiety of choice, to optimize the conjugation pattern (e.g. to ensure an optimal distribution of non-polypeptide moieties on the surface of the polypeptide and to ensure that only the attachment groups intended to be conjugated are present in the molecule) and thereby obtain a conjugate molecule which has RANKL binding activity and in addition one or more additional advantageous properties, in particular increased functional in vivo half-life and/or reduced clearance.

It will be understood that RANK and/or OPG variants according to the invention having introduced and/or removed amino acid residues comprising an attachment site may be produced by any one or more of the mutagenesis methods described herein, and similarly that variants produced by any one or more of the mutagenesis methods described herein may be conjugated to any one or more of the non-polypeptide moieties described in the following. Mutagenesis of the parent polypeptides, where applicable using more than one mutagenesis method and/or more than one round of a single mutagenesis method, may thus be performed with the aim of improving the binding affinity to RANKL, or altering the attachment sites for non-polypeptide moieties, or both.

When an attachment group for a non-polypeptide moiety is to be introduced into or removed from a RANK or OPG polypeptide by e.g. site-directed mutagenesis to produce a RANK or OPG variant in accordance with the invention, polypeptide positions that are suitable candidates for modification may be selected as follows:

The position is preferably located at the surface of the polypeptide, and more preferably occupied by an amino acid residue which has more than 25% of its side chain exposed to the solvent, preferably more than 50% of its side chain exposed to the solvent. Such positions are identified for RANK and OPG in the Examples section below.

In order to determine an optimal distribution of attachment groups, the distance between amino acid residues located at the surface of the polypeptide is calculated on the basis of a 3D structure of the polypeptide. More specifically, the distance between the CB's of the amino acid residues comprising such attachment groups, or the distance between the functional group (NZ for lysine, CG for aspartic acid, CD for glutamic acid, SG for cysteine) of one and the CB of another amino acid residue comprising an attachment group are determined. In case of glycine, CA is used instead of CB. In the polypeptides of the invention, any of said distances is preferably more than 8 Å, in particular more than 10 Å in order to avoid or reduce heterogeneous conjugation and to provide a uniform distribution of attachment groups, e.g. with the aim of epitope shielding. Further, residues that are close in sequence to each other, i.e. separated by less than three residues in the primary sequence, are potential targets for mutagenesis.

Furthermore, attachment groups located at or near the RANKL binding site of the polypeptides may advantageously be removed, preferably by substitution of the amino acid residue comprising such group.

For either introduction or removal of an attachment group, preferred substitutions include conservative substitutions or mutation to a residue in an equivalent position in a homologous sequence, e.g. a similar sequence from the TNF-receptor superfamily of structures, based on a sequence alignment.

A still further generally applicable approach for modifying a RANK or OPG polypeptide is to shield and thereby destroy or otherwise inactivate an epitope present in the parent polypeptide by conjugation to a non-polypeptide moiety. Epitopes of human RANK or OPG may be identified by use of methods known in the art, also known as epitope mapping, see e.g. Romagnoli et al., J. Biol Chem., 1999, 380(5):553-9, DeLisser H M, Methods Mol Biol, 1999, 96:11-20, Van de Water et al., Clin Immunol Immunopathol, 1997, 85(3):229-35, Saint-Remy J M, Toxicology, 1997, 119(1):77-81, and Lane D P and Stephen C W, Curr Opin Immunol, 1993, 5(2):268-71. One method is to establish a phage display library expressing random oligopeptides of e.g. 9 amino acid residues. IgG1 antibodies from specific antisera towards human RANK or OPG are purified by immunoprecipitation and the reactive phages are identified by immunoblotting. By sequencing the DNA of the purified reactive phages, the sequence of the oligopeptide can be determined followed by localization of the sequence on the 3D-structure of the polypeptide. Alternatively, epitopes can be identified according to the method described in U.S. Pat. No. 5,041,376. The thereby identified region on the structure constitutes an epitope that then can be selected as a target region for introduction of an attachment group for the non-polypeptide moiety. One or more epitopes are preferably shielded by a non-polypeptide moiety according to the present invention. Accordingly, in one embodiment, the polypeptide of the invention has at least one shielded epitope as compared to wild type human RANK or OPG. This may be done by introduction of an attachment group for a non-polypeptide moiety into a position located in the vicinity of (i.e. within 4 amino acid residues in the primary sequence or within about 10 Å in the tertiary sequence) of a given epitope. The 10 Å distance is measured between CB's (CA's in case of glycine).

Non-polypeptide Moiety of the Conjugate of the Invention

As indicated above, the non-polypeptide moiety of the conjugate of the invention is preferably selected from the group consisting of a polymer molecule, a lipophilic compound, an oligosaccharide moiety (e.g. by way of in vivo glycosylation) and an organic derivatizing agent. All of these agents may confer desirable properties to the polypeptide part of the conjugate, in particular increased functional in vivo half-life and/or increased serum half-life. The polypeptide part of the conjugate is often conjugated to only one type of non-polypeptide moiety, but may also be conjugated to two or more different types of non-polypeptide moieties, e.g. to a polymer molecule and an oligosaccharide moiety, to a lipophilic group and an oligosaccharide moiety, to an organic derivatizing agent and an oligosaccharide moiety, to a lipophilic group and a polymer molecule, etc. The conjugation to two or more different non-polypeptide moieties may be done simultaneously or sequentially.

Methods for Preparing a Conjugate of the Invention

In the following sections “Conjugation to a polymer molecule”, “Conjugation to an oligosaccharide moiety”, “Conjugation to a lipophilic compound” and “Conjugation to an organic derivatizing agent” conjugation to specific types of non-polypeptide moieties is described. In general, a polypeptide conjugate according to the invention may be produced by culturing an appropriate host cell under conditions conducive for the expression of the polypeptide, and recovering the polypeptide, wherein a) the polypeptide comprises at least one N- or O-glycosylation site and the host cell is a eukaryotic host cell capable of in vivo glycosylation, and/or b) the polypeptide is subjected to conjugation to a non-polypeptide moiety in vitro.

Conjugation to a Polymer Molecule

The polymer molecule to be coupled to the polypeptide may be any suitable polymer molecule, such as a natural or synthetic homo-polymer or heteropolymer, typically with a molecular weight in the range of about 300-100,000 Da, such as about 500-20,000 Da, more preferably in the range of about 1000-15,000 Da, even more preferably in the range of about 2000-12,000 Da, such as about 3000-10,000. When used about polymer molecules herein, the word “about” indicates an approximate average molecular weight and reflects the fact that there will normally be a certain molecular weight distribution in a given polymer preparation.

Examples of homo-polymers include a polyol (i.e. poly-OH), a polyamine (i.e. poly-NH₂) and a polycarboxylic acid (i.e. poly-COOH). A hetero-polymer is a polymer which comprises different coupling groups, such as a hydroxyl group and an amine group.

Examples of suitable polymer molecules include polymer molecules selected from the group consisting of polyalkylene oxide (PAO), including polyalkylene glycol (PAG), such as linear or branched polyethylene glycol (PEG) and polypropylene glycol (PPG), polyvinyl alcohol (PVA), poly-carboxylate, poly-(vinylpyrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule, since it has only few reactive groups capable of cross-linking compared to polysaccharides such as dextran. In particular, monofunctional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

A general method for directed conjugation of polypeptides is disclosed in WO 01/04287. PEGylated conjugates of the present invention can be prepared using this general method, which includes the following basic steps:

(a) introduction/removal of attachment groups by modification of the DNA sequence;

(b) expression of the resulting modified protein;

(c) conjugation to PEG; and

(d) screening for active or improved conjugates.

To effect covalent attachment of the polymer molecule(s) to the polypeptide, the hydroxyl end groups of the polymer molecule are provided in activated form, i.e. with reactive functional groups. Suitable activated polymer molecules are commercially available, e.g. from Shearwater Polymers, Inc., Huntsville, Ala., USA, or from PolyMASC Pharmaceuticals plc, UK. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

For PEGylation to a lysine residue, preferred activated PEG molecules suitable for conjugation include SS-PEG, NPC-PEG, aldehyde-PEG, mPEG-SPA, MPEG-SCM, mPEG-BTC from Shearwater Polymers, Inc, SC-PEG from Enzon, Inc., tresylated MPEG as described in U.S. Pat. No. 5,880,255, and oxycarbonyl-oxy-N-dicarboxyimide-PEG (U.S. Pat. No. 5,122,614).

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate). The PEGylation may be directed towards conjugation to all available attachment groups on the polypeptide (i.e. such attachment groups that are exposed at the surface of the polypeptide) or may be directed towards one or more specific attachment groups, e.g. the N-terminal amino group (U.S. Pat. No. 5,985,265). Furthermore, the conjugation may be achieved in one step or in a stepwise manner (e.g. as described in WO 99/55377).

It will be understood that the PEGylation is designed so as to produce the optimal molecule with respect to the number of PEG molecules attached, the size and form of such molecules (e.g. whether they are linear or branched), and where in the polypeptide such molecules are attached. The molecular weight of the polymer to be used will be chosen taking into consideration the desired effect to be achieved. For instance, if the primary purpose of the conjugation is to achieve a conjugate having a high molecular weight and larger size (e.g. to reduce renal clearance), one may choose to conjugate either one or a few high molecular weight polymer molecules or a number of polymer molecules with a smaller molecular weight to obtain the desired effect. Preferably, however, several polymer molecules with a smaller molecular weight will be used. When a high degree of epitope shielding is desirable, this may be obtained by use of a sufficiently high number of low molecular weight polymer molecules (e.g. with a molecular weight of about 5,000 Da) to effectively shield all or most epitopes of the polypeptide. For instance, 2-8, such as 3-6 such polymers may be used. It may also be advantageous to have a larger number of polymer molecules with a lower molecular weight (e.g. 4-6 with a MW of 5000) compared to a smaller number of polymer molecules with a higher molecular weight (e.g. 1-3 with a MW of 12,000-20,000) in terms of improving the functional in vivo half-life of the polypeptide conjugate, even where the total molecular weight of the attached polymer molecules in the two cases is the same. It is believed that the presence of a larger number of smaller polymer molecules provides the polypeptide with a larger diameter or apparent size than e.g. a single yet larger polymer molecule, at least when the polymer molecules are relatively uniformly distributed on the polypeptide surface.

While conjugation of only a single polymer molecule to a single attachment group on the protein is often not preferred, in the event that only one polymer molecule is attached, it will generally be advantageous that the polymer molecule, which may be linear or branched, has a relatively high molecular weight, e.g. about 12-20 kDa.

Normally, the polymer conjugation is performed under conditions aiming at reacting as many of the available polymer attachment groups as possible with polymer molecules. This is achieved by means of a suitable molar excess of the polymer in relation to the polypeptide. Typical molar ratios of activated polymer molecules to polypeptide are up to about 1000-1, such as up to about 200-1 or up to about 100-1. In some cases, the ratio may be somewhat lower, however, such as up to about 50-1, 10-1 or 5-1.

Subsequent to the conjugation residual activated polymer molecules are blocked according to methods known in the art, e.g. by addition of primary amine to the reaction mixture, and the resulting inactivated polymer molecules are removed by a suitable method.

In a preferred embodiment, the polypeptide conjugate of the invention comprises a PEG molecule attached to some, most or preferably substantially all of the lysine residues in the polypeptide available for PEGylation, in particular a linear or branched PEG molecule, e.g. with a molecular weight of about 1-15 kDa, typically about 2-12 kDa, such as about 3-10 kDa, e.g. about 5 or 6 kDa.

It will be understood that depending on the circumstances, e.g. the amino acid sequence of the polypeptide, the nature of the activated PEG compound being used and the specific PEGylation conditions, including the molar ratio of PEG to polypeptide, varying degrees of PEGylation may be obtained, with a higher degree of PEGylation generally being obtained with a higher ratio of PEG to polypeptide. The PEGylated polypeptides resulting from any given PEGylation process will, however, normally comprise a stochastic distribution of polypeptide conjugates having slightly different degrees of PEGylation.

In yet another embodiment, the polypeptide conjugate of the invention may comprise a PEG molecule attached to the lysine residues in the polypeptide available for PEGylation, and in addition to the N-terminal amino acid residue of the polypeptide.

Conjugate of the Invention Having a Non-lysine Residue as an Attachment Group

Amino acid residues comprising other attachment groups may be introduced into and/or removed from the RANK and/or OPG variant, using the same approach as that illustrated above by lysine residues. For instance, one or more amino acid residues comprising an acid group (glutamic acid or aspartic acid), asparagine, tyrosine or cysteine may be introduced into positions which in RANK or OPG are occupied by amino acid residues having surface exposed side chains (i.e. the positions mentioned above as being of interest for introduction of lysine residues), or removed. For PEGylation to a cysteine residue, for example, a preferred polymer molecule is VS-PEG. Introduction or removal of such amino acid residues is preferably performed by substitution. Preferably, Asp is substituted by Asn, Glu by Gln, Tyr by Phe, and Cys by Ser. Another possibility is introduction and/or removal of a histidine, e.g. by substitution with arginine.

Conjugation to an Oligosaccharide Moiety

The conjugation to an oligosaccharide moiety may take place in vivo or in vitro. In order to achieve in vivo glycosylation of a RANK and/or OPG variant of the invention comprising one or more glycosylation sites the nucleotide sequence encoding the polypeptide must be inserted in a glycosylating, eukaryotic expression host. The expression host cell may be selected from fungal (filamentous fungal or yeast), insect or animal cells or from transgenic plant cells. In one embodiment the host cell is a mammalian cell, such as a CHO cell, BHK or HEK, e.g. HEK 293, cell, or an insect cell, such as an SF9 cell, or a yeast cell, e.g. S. cerevisiae or Pichia pastoris, or any of the host cells mentioned hereinafter. Covalent in vitro coupling of glycosides (such as dextran) to amino acid residues of the polypeptide may also be used, e.g. as described in WO 87/05330 and in Aplin et al., CRC Crit Rev. Biochem., pp. 259-306, 1981.

The in vitro coupling of oligosaccharide moieties or PEG to protein- and peptide-bound Gin-residues can be carried out by transglutaminases (TG'ases). Transglutaminases catalyse the transfer of donor amine-groups to protein- and peptide-bound Gln-residues in a so-called cross-linking reaction. The donor-amine groups can be protein- or peptide-bound e.g. as the ε-amino-group in Lys-residues or can be part of a small or large organic molecule. An example of a small organic molecule functioning as an amino-donor in TG'ase-catalysed cross-linking is putrescine (1,4-diaminobutane). An example of a larger organic molecule functioning as an amino-donor in TG'ase-catalysed cross-linking is an amine-containing PEG (Sato et al., Biochemistry 35, 13072-13080).

TG'ases are in general highly specific enzymes, and not every Gln-residue exposed on the surface of a protein is accessible to TG'ase-catalysed cross-linking to amino-containing substances. On the contrary, only a few Gln-residues function naturally as TG'ase substrates, but the exact parameters governing which Gln-residues are good TG'ase substrates remain unknown. Thus, in order to render a protein susceptible to TG'ase-catalysed cross-linking reactions it is often a prerequisite to add at convenient positions stretches of amino acid sequence known to function very well as TG'ase substrates. Several amino acid sequences are known to be or to contain excellent natural TG'ase substrates e.g. substance P, elafin, fibrinogen, fibronectin, α₂-plasmin inhibitor, α-caseins, and β-caseins.

Conjugation to a Lipophilic Compound

The polypeptide and the lipophilic compound may be conjugated to each other, either directly or by use of a linker. The lipophilic compound may be a natural compound such as a saturated or unsaturated fatty acid, a fatty acid diketone, a terpene, a prostaglandin, a vitamin, a carotenoid or steroid, or a synthetic compound such as a carbon acid, an alcohol, an amine and sulphonic acid with one or more alkyl, aryl, alkenyl or other multiple unsaturated compounds. The conjugation between the polypeptide and the lipophilic compound, optionally through a linker, may be done according to methods known in the art, e.g. as described by Bodanszky in Peptide Synthesis, John Wiley, New York, 1976 and in WO 96/12505.

Coupling to an Organic Derivatizing Agent

Covalent modification of the polypeptide may be performed by reacting one or more attachment groups of the polypeptide with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonate at pH 5.5-7.0 because this agent is relatively specific for the histidyl side chain. Para-bromophenacyl bromide is also useful. The reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing a-amino-containing residues include imidoesters such as methyl picolinimidate, pyridoxal phosphate, pyridoxal, chloroborohydride, trinitrobenzenesulfonic acid, O-methylisourea, 2,4-pentanedione and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group.

Furthermore, these reagents may react with the groups of lysine as well as the arginine guanidino group. Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Blocking of the Functional Site

It has been reported that excessive polymer conjugation can lead to a loss of activity of the polypeptide to which the polymer is conjugated. This problem can be eliminated by e.g. removal of attachment groups located at the functional site or by blocking the functional site prior to conjugation so that the functional site is blocked during conjugation. The latter strategy constitutes a further embodiment of the invention (the first strategy being exemplified further above, e.g. by removal of lysine residues which may be located close to the functional site). More specifically, according to the second strategy the conjugation between the polypeptide and the non-polypeptide moiety is conducted under conditions where the functional site of the polypeptide is blocked by a helper molecule capable of binding to the functional site of the polypeptide.

Preferably, the helper molecule is one which specifically recognizes a functional site of the polypeptide. Alternatively, the helper molecule may be an antibody, in particular a monoclonal antibody recognizing the polypeptide. In particular, the helper molecule may be a neutralizing monoclonal antibody.

The polypeptide is allowed to interact with the helper molecule before effecting conjugation. This ensures that the functional site of the polypeptide is shielded or protected and consequently unavailable for derivatization by the non-polypeptide moiety such as a polymer. Following its elution from the helper molecule, the conjugate between the non-polypeptide moiety and the polypeptide can be recovered with at least a partially preserved functional site.

The subsequent conjugation of the polypeptide having a blocked functional site to a polymer, a lipophilic compound, an oligosaccharide moiety, an organic derivatizing agent or any other compound is conducted in the normal way, e.g. as described in the sections above entitled “Conjugation to . . . ”.

Irrespective of the nature of the helper molecule to be used to shield the functional site of the polypeptide from conjugation, it is desirable that the helper molecule is free of or comprises only a few attachment groups for the non-polypeptide moiety of choice in part(s) of the molecule where the conjugation to such groups would hamper desorption of the conjugated polypeptide from the helper molecule. Hereby, selective conjugation to attachment groups present in non-shielded parts of the polypeptide can be obtained and it is possible to re-use the helper molecule for repeated cycles of conjugation. For instance, if the non-polypeptide moiety is a polymer molecule such as PEG, which has the epsilon amino group of a lysine or N-terminal amino acid residue as an attachment group, it is desirable that the helper molecule is substantially free of conjugatable epsilon amino groups, preferably free of any epsilon amino groups. Accordingly, in a preferred embodiment the helper molecule is a protein or peptide capable of binding to the functional site of the polypeptide, which protein or peptide is free of any conjugatable attachment groups for the non-polypeptide moiety of choice.

Of particular interest in connection with the embodiment of the present invention wherein the polypeptide conjugates are prepared from a diversified population of nucleotide sequences encoding a polypeptide of interest, the blocking of the functional group is effected in microtiter plates prior to conjugation, for instance by plating the expressed polypeptide variant in a microtiter plate containing an immobilized blocking group such as a receptor, an antibody or the like.

In a further embodiment the helper molecule is first covalently linked to a solid phase such as column packing materials, for instance Sephadex or agarose beads, or a surface, e.g. a reaction vessel. Subsequently, the polypeptide is loaded onto the column material carrying the helper molecule and conjugation carried out according to methods known in the art, e.g. as described in the sections above entitled “Conjugation to . . . ”. This procedure allows the polypeptide conjugate to be separated from the helper molecule by elution. The polypeptide conjugate is eluted by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule may be derivatised with a second molecule (e.g. biotin) that can be recognized by a specific binder (e.g. streptavidin). The specific binder may be linked to a solid phase, thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate may be released from the helper molecule in any appropriate fashion. Deprotection may be achieved by providing conditions in which the helper molecule dissociates from the functional site of the polypeptide to which it is bound. For instance, a complex between an antibody to which a polymer is conjugated and an anti-idiotypic antibody can be dissociated by adjusting the pH to an acid or alkaline pH.

Conjugation of a Tagged Polypeptide

In an alternative embodiment the polypeptide is expressed as a fusion protein with a tag, i.e. an amino acid sequence or peptide stretch made up of typically 1-30, such as 1-20 amino acid residues. Besides allowing for fast and easy purification, the tag is a convenient tool for achieving conjugation between the tagged polypeptide and the non-polypeptide moiety. In particular, the tag may be used for achieving conjugation in microtiter plates or other carriers, such as paramagnetic beads, to which the tagged polypeptide can be immobilised via the tag. The conjugation to the tagged polypeptide in e.g. microtiter plates has the advantage that the tagged polypeptide can be immobilised in the microtiter plates directly from the culture broth (in principle without any purification) and subjected to conjugation. Thereby, the total number of process steps (from expression to conjugation) can be reduced. Furthermore, the tag may function as a spacer molecule, ensuring an improved accessibility to the immobilised polypeptide to be conjugated. The conjugation using a tagged polypeptide may be to any of the non-polypeptide moieties disclosed herein, e.g. to a polymer molecule such as PEG.

The identity of the specific tag to be used is not critical as long as the tag is capable of being expressed with the polypeptide and is capable of being immobilised on a suitable surface or carrier material. A number of suitable tags are commercially available, e.g. from Unizyme Laboratories, Denmark. For instance, the tag may consist of any of the following sequences:

His-His-His-His-His-His

Met-Lys-His-His-His-His-His-His

Met-Lys-His-His-Ala-His-His-Gln-His-His

Met-Lys-His-Gln-His-Gln-His-Gln-His-Gln-His-Gln-His-Gln

Met-Lys-His-Gin-His-Gln-His-Gln-His-Gln-His-Gln-His-Gln-Gln

or any of the following:

EQKLISEEDL (a C-terminal tag described in Mol. Cell. Biol. 5:3610-16, 1985)

DYKDDDDK (a C- or N-terminal tag)

YPYDVPDYA

Antibodies against the above tags are commercially available, e.g. from ADI, Aves Lab and Research Diagnostics. The subsequent cleavage of the tag from the polypeptide may be achieved by use of commercially available enzymes.

Methods for Preparing a Polypeptide of the Invention or the Polypeptide Part of the Conjugate of the Invention

The polypeptide of the present invention or the polypeptide part of a conjugate of the invention, optionally in glycosylated form, may be produced by any suitable method known in the art. Such methods include constructing a nucleotide sequence encoding the polypeptide and expressing the sequence in a suitable transformed or transfected host. However, polypeptides of the invention may be produced, albeit less efficiently, by chemical synthesis or a combination of chemical synthesis or a combination of chemical synthesis and recombinant DNA technology.

A nucleotide sequence encoding a polypeptide or the polypeptide part of a conjugate of the invention may be constructed by isolating or synthesizing a nucleotide sequence encoding the parent polypeptide, and then changing the nucleotide sequence so as to effect introduction (i.e. insertion or substitution) or deletion (i.e. removal or substitution) of the relevant amino acid residue(s).

The nucleotide sequence is, in one embodiment, conveniently modified by site-directed mutagenesis in accordance with conventional methods. Alternatively, the nucleotide sequence is prepared by chemical synthesis, e.g. by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by PCR, ligation or ligation chain reaction (LCR) (Barany, PNAS 88:189-193, 1991). The individual oligonucleotides typically contain 5′ or 3′ overhangs for complementary assembly.

Alternative nucleotide sequence modification methods are available for producing polypeptide variants for high throughput screening, for instance methods which involve homologous cross-over such as disclosed in U.S. Pat. No. 5,093,257, and methods which involve gene shuffling, i.e. recombination between two or more homologous nucleotide sequences resulting in new nucleotide sequences having a number of nucleotide alterations when compared to the starting nucleotide sequences. Gene shuffling (also known as DNA shuffling) involves one or more cycles of random fragmentation and reassembly of the nucleotide sequences, followed by screening to select nucleotide sequences encoding polypeptides with desired properties. In order for homology-based nucleic acid shuffling to take place, the relevant parts of the nucleotide sequences are preferably at least 50% identical, such as at least 60% identical, more preferably at least 70% identical, such as at least 80% identical. The recombination can be performed in vitro or in vivo. Shuffling techniques suitable for preparing RANK and/or OPG variants of the invention are described in detail below.

Dimerization of the Compounds

It has been reported that dimers of OPG bind to RANKL with a higher affinity than monomer OPG (Tomoyasu, et al., (1998) Biochem.Biophys.Res.Comm. 245, 382-7). Therefore, in one embodiment, RANK and/or OPG variants of the invention may be produced as dimeric or even as oligomeric single-chain molecules, with two, three or possibly more monomers joined typically by a peptide bond or a peptide linker, or e.g. by means of a PEG molecule.

Dimerisation can for example be achieved by producing the compound as a fusion protein with the Fc-portion of Ig gamma 1 (GenPept accession No. M87789.1). The molecules can be expressed as fusion proteins with a C-terminal Fc-part or with a N-termiinal Fc-part.

Dimerisation can also be achieved by fusing the product candidate to a GCN4 leucine zipper, which has been reported to induce dimerisation of fusion proteins (Doñate, et al., (2000) Biochemistry, 39 11467-76).

Alternatively, dimeric molecules may be produced by mutagenizing one of the last five, or alternatively one of the first five amino acid residues to a cysteine residue. An unpaired cysteine residue of the purified compound can then be attached to a “di-active” PEG group by using existing thiol reactive attachment groups. Alternatively, dimeric molecules can be produced by inserting two candidate molecules (identical or even different) in-frame with a suitable flexible polypeptide linker in an appropriate expression vector.

For single-chain constructs, the linker peptide will often predominantly include the amino acid residues Gly, Ser, Ala and/or Thr. Such a linker typically comprises 1-30 amino acid residues, such as a sequence of about 2-20 or 3-15 amino acid residues. The amino acid residues selected for inclusion in the linker peptide should exhibit properties that do not interfere significantly with the activity of the polypeptide. Thus, the linker peptide should on the whole not exhibit a charge which would be inconsistent with the desired RANKL binding activity, or interfere with internal folding, or form bonds or other interactions with amino acid residues in one or more of the subunits which would seriously impede the binding of the dimeric or multimeric polypeptide.

Specific linkers for use in the present invention may be designed on the basis of known naturally occurring as well as artificial polypeptide linkers (see, e.g., Hallewell et al. (1989), J. Biol. Chem. 264, 5260-5268; Alfthan et al. (1995), Protein Eng. 8, 725-731; Robinson & Sauer (1996), Biochemistry 35, 109-116; Khandekar et al. (1997), J. Biol. Chem. 272, 32190-32197; Fares et al. (1998), Endocrinology 139, 2459-2464; Smallshaw et al. (1999), Protein Eng. 12, 623-630; U.S. Pat. No. 5,856,456). For instance, linkers used for creating single-chain antibodies, e.g. a 15mer consisting of three repeats of a Gly-Gly-Gly-Gly-Ser amino acid sequence ((Gly₄Ser)₃), are contemplated to be useful. Furthermore, phage display technology as well as selective infective phage technology can be used to diversify and select appropriate lo linker sequences (Tang et al., J. Biol. Chem. 271, 15682-15686, 1996; Hennecke et al. (1998), Protein Eng. 11, 405-410). Also, Arc repressor phage display has been used to optimize the linker length and composition for increased stability of a single-chain protein (Robinson and Sauer (1998), Proc. Natl. Acad. Sci. USA 95, 5929-5934). Another way of obtaining a suitable linker is by optimizing a simple linker, e.g. ((Gly₄Ser)ₙ), through random mutagenesis. The is linker may e.g. be (Gly₄Ser)ₙ or (Gly₃Ser)ₙ where n is 1, 2, 3 or 4.

Shuffling/Recombination

It has been shown that shuffling two or more molecules can alter certain characteristics and readily improve many characteristics of a pool of molecules. One suitable technique for shuffling RANK or OPG encoding sequences is “family shuffling”.

Family shuffling of OPG encoding cDNA sequences may be performed by cloning OPG from different animal species, in particular mammals, for example mouse, rat, dog, cat, sheep, goat, cow, horse, rabbit, hamster, guinea pig, etc, and preferably primates, including humans as well as non-human primates, for example chimpanzee, gorilla, orangutan, baboon, mandrill, monkey, bonobo, marmoset, macaque, lemur, gibbon, shrew, siamang, tamarin, etc. It will be clear that OPG-encoding sequences from other species can also be used, including other non-mammal species, both vertebrates and invertebrates, for example trout or other species of fish. As an example, it is contemplated that OPG from e.g. humans, mouse, rat and trout will be cloned or produced from synthetic oligos. These cDNA sequences will then be employed in a series of family shuffling reactions as detailed below.

Family shuffling of RANK-encoding cDNA sequences may similarly be performed by cloning RANK from different species, which as in the case of OPG may be from different animal species, including non-mammal species as well as mammalian species, but preferably mammalian species and in particular primates. RANK sequences from any of the species mentioned above for OPG may be used for RANK shuffling. As an example, it is contemplated that RANK from e.g. humans and mouse will be cloned or produced from synthetic oligos. These cDNA sequences will then be employed in a series of family shuffling reactions as detailed below.

Shuffling of the cDNA sequences encoding the RANKL binding parts of human OPG and human RANK may also be employed in a series of doped oligonucleotide shuffling reactions as detailed below. A selected fraction of the resulting molecules from these doped oligonucleotide shuffling reactions are subsequently employed in a series of shuffling reactions with or without the native or mutated OPG cDNA and/or the native or mutated RANKL cDNA from any or all of the species mentioned above.

The doped oligonucleotide shuffling reactions may be performed sequentially covering all residues of the 179 amino acid alignment shown in FIG. 4b. Preferably, the shuffling reactions are performed on stretches directly involved in ligand binding and the amino acid residues flanking these amino acids. The highest priority regions include the stretches 7-18, 24-32, 45-73, 90-108, 123-125, and 137-139.

Shuffling of the cDNA sequences encoding the RANKL binding parts of human OPG and human RANK may also be employed in a series of “cross-over oligonucleotide shuffling” reactions as detailed below. A selected fraction of the resulting molecules from these cross-over oligonucleotide shuffling reactions are subsequently employed in a series of shuffling reactions with or without the native or mutated OPG cDNA and/or the native or mutated RANKL cDNA from any or all of the species mentioned above.

In crossover oligonucleotide mediated shuffling, oligonucleotides corresponding to a family of related homologous nucleic acids (e.g., as applied to the present invention, inter-specific or allelic variants of a RANK and/or OPG encoding nucleic acid) which are recombined to produce selectable nucleic acids. This format is described in detail in Crameri et al. “OLIGONUCLEOTIDE MEDIATED NUCLEIC ACID RECOMBINATION” filed Feb. 5, 1999, U.S. Ser. No. 60/118,813 and Crameri et al. “OLIGONUCLEOTIDE MEDIATED NUCLEIC ACID RECOMBINATION” filed Jun. 24, 1999, U.S. Ser. No. 60/141,049. This technique can be used to recombine homologous or even non-homologous nucleic acid sequences.

One advantage of the oligonucleotide-mediated recombination is the ability to recombine homologous nucleic acids with low sequence similarity, or even non-homologous nucleic acids. In these low-homology oligonucleotide shuffling methods, one or more set of fragmented nucleic acids are recombined, e.g., with a with a set of crossover diversity oligonucleotides. Each of these crossover oligonucleotides have a plurality of sequence diversity domains corresponding to a plurality of sequence diversity domains from homologous or non-homologous nucleic acids with low sequence similarity. The fragmented oligonucleotides, which are derived by comparison to one or more homologous or non-homologous nucleic acids, can hybridize to one or more region of the crossover oligos, facilitating recombination.

Formats for Sequence Recombination

Preferred methods of the invention for producing novel RANKL-binding proteins entail performing recombination (“shuffling”) and screening or selection to “evolve” individual genes, whole plasmids or viruses, multigene clusters, or even whole genomes (Stemmer, Bio/Technology 13:549-553 (1995)) for improving the RANK or OPG pharmaceutical properties. Reiterative cycles of recombination and screening/selection can be performed to further evolve the nucleic acids of interest. Such techniques do not require the extensive analysis and computation required by conventional methods for polypeptide engineering. Shuffling allows the recombination of large numbers of mutations in a minimum number of selection cycles, in contrast to natural pair-wise recombination events (e.g., as occur during sexual replication). Thus, the sequence recombination techniques described herein provide particular advantages in that they provide recombination between mutations in any or all of these, thereby providing a very fast way of exploring the manner in which different combinations of mutations can affect a desired result.

In some instances, however, structural and/or functional information is available which, although not required for sequence recombination, provides opportunities for modification of the technique. Such information, including the information provided above regarding RANK and OPG based on structural alignments of these polypeptides, can also be used for site directed or random mutagenesis, e.g. for mutating desired amino acid residues in order to introduce or remove attachment sites for PEGylation or glycosylation sites.

A variety of nucleic acid shuffling protocols are available and fully described in the art. Descriptions of a variety of shuffling methods for generating modified nucleic acid sequences for use in the methods of the present invention include the following publications and the references cited therein: Stemmer et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Additional details regarding DNA shuffling methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/13487 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” and WO 98/41622 by Borchert et al., “Method for Constructing a Library Using DNA Shuffling.”

Certain U.S. patent applications provide additional details regarding shuffling methods, including “SHUFFLING OF CODON ALTERED GENES” by Patten et al. filed Sep. 29, 1998, (U.S. Ser. No. 60/102,362), Jan. 29, 1999 (U.S. Ser. No. 60/117,729), and Sep. 28, 1999, (U.S. Ser. No. 09/407,800); “EVOLUTION OF WHOLE CELLS AND ORGANISMS BY RECURSIVE SEQUENCE RECOMBINATION”, by del Cardayre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Feb. 5, 1999 (U.S. Ser. No. 60/118,813), Jun. 24, 1999 (U.S. Ser. No. 60/141,049), and Sep. 28, 1999 (U.S. Ser. No. 09/408,392); “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118854) and Oct. 12, 1999 (U.S. Ser. No. 09/416,375); and “SINGLE-STRANDED NUCLEIC ACID TEMPLATE-MEDIATED RECOMBINATION AND NUCLEIC ACID FRAGMENT ISOLATION” by Affholter, U.S. Ser. No. 60/186,482 filed Mar. 2, 2000.

In brief, a variety of shuffling formats are applicable to the present invention and set forth, e.g., in the references above. The following exemplify some of the different types of formats. First, nucleic acids can be recombined in vitro by any of a variety of techniques discussed in the references above, including e.g., DNAse digestion of nucleic acids to be recombined followed by ligation and/or PCR reassembly of the nucleic acids. Second, nucleic acids can be recursively recombined in vivo, e.g., by allowing recombination to occur between nucleic acids in cells. Third, whole genome recombination methods can be used in which whole genomes of cells or other organisms are recombined, optionally including spiking of the genomic recombination mixtures with desired library components (e.g., genes corresponding to the pathways of the present invention). Fourth, synthetic recombination methods can be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Fifth, in silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. Any of the preceding general recombination formats can be practiced in a reiterative fashion to generate a more diverse set of recombinant nucleic acids. Sixth, methods of accessing natural diversity, e.g. by hybridization of diverse nucleic acids or nucleic acid fragments to single-stranded templates, followed by polymerization and/or ligation to regenerate full-length sequences, optionally followed by degradation of the templates and recovery of the resulting modified nucleic acids, can be used.

The above references provide these and other basic recombination formats as well as many modifications of these formats. Regardless of the shuffling format which is used, the nucleic acids of the invention can be recombined (with each other, or with related or even unrelated sequences) to produce a diverse set of recombinant nucleic acids, including, e.g., sets of homologous nucleic acids.

Following recombination, any nucleic acids which are produced can be selected for a desired activity. In the context of the present invention, this can include testing for and identifying any activity that can be detected e.g., in an automatable format, by any of the assays in the art. A variety of related (or even unrelated) properties can be assayed for, using any available assay.

DNA mutagenesis and shuffling provide a robust, widely applicable means of generating diversity useful for the engineering of proteins, pathways, cells and organisms with improved characteristics. In addition to the basic formats described above, it is sometimes desirable to combine shuffling methodologies with other techniques for generating diversity. In conjunction with (or separately from) shuffling methods, a variety of diversity generation methods can be practiced and the results (i.e. diverse populations of nucleic acids) screened for in the systems of the invention. Additional diversity can be introduced by methods which result in the alteration of individual nucleotides or groups of contiguous or non-contiguous nucleotides, i.e. mutagenesis methods. Many mutagenesis methods are found in the above-cited references; additional details regarding mutagenesis methods can be found in the references listed below.

Mutagenesis methods include, for example, those described in PCT/US98/05223; Publ. No. WO98/42727; site-directed mutagenesis (Ling et al. (1997) “Approaches to DNA mutagenesis: an overview” Anal. Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

In one aspect of the present invention, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same vial, with the products of one reaction priming the products of another reaction. Sexual PCR mutagenesis can be used in which homologous recombination occurs between DNA molecules of different but related DNA sequence in vitro, by random fragmentation of the DNA molecule based on sequence homology, followed by fixation of the crossover by primer extension in a PCR reaction. This process is described in the references above, e.g., in Stemmer (1994) Proc. Natl. Acad. Sci. USA 91:10747-10751. Recursive ensemble mutagenesis can be used in which an algorithm for protein mutagenesis is used to produce diverse populations of phenotypically related mutants whose members differ in amino acid sequence. This method uses a feedback mechanism to control successive rounds of combinatorial cassette mutagenesis. Examples of this approach are found in Arkin & Youvan (1992) Proc. Natl. Acad. Sci. USA 89:7811-7815.

As noted, oligonucleotide directed mutagenesis can be used in a process which allows for the generation of site-specific mutations in any nucleic acid sequence of interest. Examples of such techniques are found in the references above and, e.g., in Reidhaar-Olson et al. (1988) Science, 241:53-57. Similarly, cassette mutagenesis can be used in a process which replaces a small region of a double stranded DNA molecule with a synthetic oligonucleotide cassette that differs from the native sequence. The oligonucleotide can contain, e.g., completely and/or partially randomized native sequence(s).

In vivo mutagenesis can be used in a process of generating random mutations in any cloned DNA of interest which involves the propagation of the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA.

Exponential ensemble mutagenesis can be used for generating combinatorial libraries with a high percentage of unique and functional mutants, where small groups of residues are randomized in parallel to identify, at each altered position, amino acids which lead to functional proteins. Examples of such procedures are found in Delegrave & Youvan (1993) Biotechnology Research 11:1548-1552. Similarly, random and site-directed mutagenesis can be used. Examples of such procedures are found in Arnold (1993) Current Opinion in Biotechnology 4:450-455.

Kits for mutagenesis are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

Any of the described shuffling or mutagenesis techniques can be used in conjunction with procedures which introduce additional diversity into a genome, e.g. a bacterial, fungal, animal or plant genome. For example, in addition to the methods above, techniques have been proposed which produce nucleic acid multimers suitable for transformation into a variety of species (see, e.g., Schellenberger, U.S. Pat. No. 5,756,316 and the references above). When such multimers consist of genes that are divergent with respect to one another, (e.g., derived from natural diversity or through application of site directed mutagenesis, error prone PCR, passage through mutagenic bacterial strains, and the like), are transformed into a suitable host, this provides a source of nucleic acid diversity for DNA diversification.

Multimers transformed into host species are suitable as substrates for in vivo shuffling protocols. Alternatively, a multiplicity of polynucleotides sharing regions of partial sequence similarity can be transformed into a host species and recombined in vivo by the host cell. Subsequent rounds of cell division can be used to generate libraries, members of which, comprise a single, homogenous population of monomeric or pooled nucleic acid. Alternatively, the monomeric nucleic acid can be recovered by standard techniques and recombined in any of the described shuffling formats.

Shuffling formats employing chain termination methods have also been proposed (see e.g., U.S. Pat. No. 5,965,408 and the references above). In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are chimeric with respect to the starting population of DNA molecules. Optionally, the products or partial pools of the products can be amplified at one or more stages in the process. Polynucleotides produced by a chain termination method, such as described above are suitable substrates for DNA shuffling according to any of the described formats.

Diversity can be further increased by using non-homology based shuffling methods (which, as set forth in the above publications and applications can be homology or non-homology based, depending on the precise format). For example, incremental truncation for the creation of hybrid enzymes (ITCHY) described in Ostermeier et al. (1999) “A combinatorial approach to hybrid enzymes independent of DNA homology” Nature Biotech 17:1205, can be used to generate an initial a shuffled library which can optionally serve as a substrate for one or more rounds of in vitro or in vivo shuffling methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Methods for generating multispecies expression libraries have been described (e.g., U.S. Pat. Nos. 5,783,431; 5,824,485 and the references above) and their use to identify protein activities of interest has been proposed (U.S. Pat. No. 5,958,672 and the references above). Multispecies expression libraries are, in general, libraries comprising cDNA or genomic sequences from a plurality of species or strains, operably linked to appropriate regulatory sequences, in an expression cassette. The cDNA and/or genomic sequences are optionally randomly concatenated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

In some applications, it is desirable to preselect or prescreen libraries (e.g., an amplified library, a genomic library, a cDNA library, a normalized library, etc.) or other substrate nucleic acids prior to shuffling, or to otherwise bias the substrates towards nucleic acids that encode functional products (shuffling procedures can also, independently have these effects). For example, in the case of antibody engineering, it is possible to bias the shuffling process toward antibodies with functional antigen binding sites by taking advantage of in vivo recombination events prior to DNA shuffling by any described method. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to DNA shuffling according to any of the methods described herein.

Libraries can be biased towards nucleic acids which encode proteins with desirable activities. For example, after identifying a clone from a library which exhibits a specified activity, the clone can be mutagenized using any known method for introducing DNA alterations, including, but not restricted to, DNA shuffling. A library comprising the mutagenized homologues is then screened for a desired activity, which can be the same as or different from the initially specified activity. An example of such a procedure is proposed in U.S. Pat. No. 5,939,250. Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom.

Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in a shuffling format that employs a single-stranded template. Such single-stranded template shuffling formats are described, for example, in WO 98/27230, “Methods and Compositions for Polypeptide Engineering” by Patten et al.; U.S. Ser. No. 60/186,482 filed Mar. 2, 2000, “Single-Stranded Nucleic Acid Template-Mediated Recombination and Nucleic Acid Fragment Isolation” by Affholter; WO 00/00632, “Methods for Generating Highly Diverse Libraries” by Wagner et al.; and WO 00/09679, “Methods for Obtaining in Vitro Recombined Polynucleotide Sequence Banks and Resulting Sequences.” In one such method the fragment population derived the genomic library(ies) is annealed with partial, or, often approximately full length ssDNA or RNA corresponding to the opposite strand. Assembly of complex chimeric genes from this population is the mediated by nuclease-base removal of non-hybridizing fragment ends, polymerization to fill gaps between such fragments and subsequent single stranded ligation. The parental strand can be removed by digestion (if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps.

In one approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further shuffling using any of the shuffling reactions described herein.

It will further be appreciated that any of the above described techniques suitable for enriching a library prior to shuffling can be used to screen the products generated by the methods of DNA shuffling.

The shuffling of a single gene and the shuffling of a family of genes provide two of the most powerful methods available for improving and “migrating” (gradually changing the type of reaction, substrate or activity of a selected protein) the functions of proteins. When shuffling a family of genes, homologous sequences, e.g., from different species or chromosomal positions, are recombined. In single gene shuffling, a single sequence is mutated or otherwise altered and then recombined. These formats share some common principles.

The breeding procedure starts with at least two substrates that generally show substantial sequence identity to each other (i.e., at least about 30%, 50%, 70%, 80% or 90% sequence identity), but differ from each other at certain positions. The difference can be any type of mutation, for example, substitutions, insertions and deletions. Often, different segments differ from each other in about 5-20 positions. For recombination to generate increased diversity relative to the starting materials, the starting materials must differ from each other in at least two nucleotide positions. That is, if there are only two substrates, there should be at least two divergent positions. If there are three substrates, for example, one substrate can differ from the second at a single position, and the second can differ from the third at a different single position. The starting DNA segments can be natural variants of each other, for example, allelic or species variants. The segments can also be from nonallelic genes showing some degree of structural and usually functional relatedness (e.g., different genes within a superfamily, such as the RANK and OPG and TNF-alpha receptor genes). The starting DNA segments can also be induced variants of each other. For example, one DNA segment can be produced by error-prone PCR replication of the other, or by substitution of a mutagenic cassette. Induced mutants can also be prepared by propagating one (or both) of the segments in a mutagenic strain. In these situations, strictly speaking, the second DNA segment is not a single segment but a large family of related segments. The different segments forming the starting materials are often the same length or substantially the same length. However, this need not be the case; for example; one segment can be a subsequence of another. The segments can be present as part of larger molecules, such as vectors, or can be in isolated form.

The starting DNA segments are recombined by any of the sequence recombination formats provided herein to generate a diverse library of recombinant DNA segments. Such a library can vary widely in size from having fewer than 10 to more than 10⁵, 10⁷, 10⁹, 10¹² or more members. In some embodiments, the starting segments and the recombinant libraries generated will include full-length coding sequences and any essential regulatory sequences, such as a promoter and polyadenylation sequence, required for expression. In other embodiments, the recombinant DNA segments in the library can be inserted into a common vector providing sequences necessary for expression before performing screening/selection.

1. Use of Restriction Enzyme Sites to Recombine Mutations

In some situations it is advantageous to use restriction enzyme sites in nucleic acids to direct the recombination of mutations in a nucleic acid sequence of interest. These techniques are particularly preferred in the evolution of fragments that cannot readily be shuffled by existing methods due to the presence of repeated DNA or other problematic primary sequence motifs. These situations also include recombination formats in which it is preferred to retain certain sequences unmutated. The use of restriction enzyme sites is also preferred for shuffling large fragments (typically greater than 10 kb), such as gene clusters that cannot be readily shuffled and “PCR-amplified” because of their size. Although fragments up to 50 kb have been reported to be amplified by PCR (Barnes, Proc. Natl. Acad. Sci. U.S.A. 91:2216-2220 (1994)), it can be problematic for fragments over 10 kb, and thus alternative methods for shuffling in the range of 10-50 kb and beyond are preferred. Preferably, the restriction endonucleases used are of the Class II type (Sambrook, Ausubel and Berger, supra) and of these, preferably those which generate nonpalindromic sticky end overhangs such as Alwn I, Sfi I or BstX1. These enzymes generate nonpalindromic ends that allow for efficient ordered reassembly with DNA ligase. Typically, restriction enzyme (or endonuclease) sites are identified by conventional restriction enzyme mapping techniques (Sambrook, Ausubel, and Berger, supra.), by analysis of sequence information for that gene, or by introduction of desired restriction sites into a nucleic acid sequence by synthesis (i.e. by incorporation of silent mutations).

The DNA substrate molecules to be digested can either be from in vivo replicated DNA, such as a plasmid preparation, or from PCR amplified nucleic acid fragments harboring the restriction enzyme recognition sites of interest, preferably near the ends of the fragment. Typically, at least two variants of a gene of interest, each having one or more mutations, are digested with at least one restriction enzyme determined to cut within the nucleic acid sequence of interest. The restriction fragments are then joined with DNA ligase to generate full length genes having shuffled regions. The number of regions shuffled will depend on the number of cuts within the nucleic acid sequence of interest. The shuffled molecules can be introduced into cells as described above and screened or selected for a desired property as described herein. Nucleic acids can then be isolated from pools (libraries), or clones having desired properties and subjected to the same procedure until a desired degree of improvement is obtained.

In some embodiments, at least one DNA substrate molecule or fragment thereof is isolated and subjected to mutagenesis. In some embodiments, the pool or library of religated restriction fragments are subjected to mutagenesis before the digestion-ligation process is repeated. “Mutagenesis” as used herein includes such techniques known in the art as PCR mutagenesis, oligonucleotide-directed mutagenesis, site-directed mutagenesis, etc., and recursive sequence recombination by any of the techniques described herein.

2. Reassembly PCR

A further technique for recombining mutations in a nucleic acid sequence utilizes “reassembly PCR.” This method can be used to assemble multiple segments that have been separately evolved into a full length nucleic acid template such as a gene. This technique is performed when a pool of advantageous mutants is known from previous work or has been identified by screening mutants that may have been created by any mutagenesis technique known in the art, such as PCR mutagenesis, cassette mutagenesis, doped oligo mutagenesis, chemical mutagenesis, or propagation of the DNA template in vivo in mutator strains. Boundaries defining segments of a nucleic acid sequence of interest preferably lie in intergenic regions, introns, or areas of a gene not likely to have mutations of interest. Preferably, oligonucleotide primers (oligos) are synthesized for PCR amplification of segments of the nucleic acid sequence of interest, such that the sequences of the oligonucleotides overlap the junctions of two segments. The overlap region is typically about 10 to 100 nucleotides in length. Each of the segments is amplified with a set of such primers. The PCR products are then “reassembled” according to assembly protocols such as those discussed herein to assemble randomly fragmented genes. In brief, in an assembly protocol the PCR products are first purified away from the primers, by, for example, gel electrophoresis or size exclusion chromatography. Purified products are mixed together and subjected to about 1-10 cycles of denaturing, reannealing, and extension in the presence of polymerase and deoxynucleoside triphosphates (dNTP's) and appropriate buffer salts in the absence of additional primers (“self-priming”). Subsequent PCR with primers flanking the gene are used to amplify the yield of the fully reassembled and shuffled genes.

In some embodiments, the resulting reassembled genes are subjected to mutagenesis before the process is repeated.

In a further embodiment, the PCR primers for amplification of segments of the nucleic acid sequence of interest are used to introduce variation into the gene of interest as follows. Mutations at sites of interest in a nucleic acid sequence are identified by screening or selection, by sequencing homologues of the nucleic acid sequence, and so on. Oligonucleotide PCR primers are then synthesized which encode wild type or mutant information at sites of interest. These primers are then used in PCR mutagenesis to generate libraries of full length genes encoding permutations of wild type and mutant information at the designated positions. This technique is typically advantageous in cases where the screening or selection process is expensive, cumbersome, or impractical relative to the cost of sequencing the genes of mutants of interest and synthesizing mutagenic oligonucleotides.

3. Site Directed Mutagenesis (SDM) with Oligonucleotides Encoding Homologue Mutations Followed by Shuffling

In some embodiments of the invention, sequence information from one or more substrate sequences is added to a given “parental” sequence of interest, with subsequent recombination between rounds of screening or selection. Typically, this is done with site-directed mutagenesis performed by techniques well known in the art (e.g., Berger, Ausubel and Sambrook, supra.) with one substrate as template and oligonucleotides encoding single or multiple mutations from other substrate sequences, e.g. homologous genes. After screening or selection for an improved phenotype of interest, the selected recombinant(s) can be further evolved using RSR techniques described herein. After screening or selection, site-directed mutagenesis can be done again with another collection of oligonucleotides encoding homologue mutations, and the above process repeated until the desired properties are obtained.

When the difference between two homologues is one or more single point mutations in a codon, degenerate oligonucleotides can be used that encode the sequences in both homologues. One oligonucleotide can include many such degenerate codons and still allow one to exhaustively search all permutations over that block of sequence.

When the homologue sequence space is very large, it can be advantageous to restrict the search to certain variants. Thus, for example, computer modeling tools (Lathrop et al., J. Mol. Biol. 255:641-665 (1996)) can be used to model each homologue mutation onto the target protein and discard any mutations that are predicted to grossly disrupt structure and function.

4. In Vitro Nucleic Acid Shuffling Formats

In one embodiment for shuffling DNA sequences in vitro, the initial substrates for recombination are a pool of related sequences, e.g., different variant forms, as homologs from different individuals, strains, or species of an organism, or related sequences from the same organism, as allelic variations. The sequences can be DNA or RNA and can be of various lengths depending on the size of the gene or DNA fragment to be recombined or reassembled. Preferably the sequences are from 50 base pairs (bp) to 50 kilobases (kb).

The pool of related substrates are converted into overlapping fragments, e.g., from about 5 bp to 5 kb or more. Often, for example, the size of the fragments is from about 10 bp to 1000 bp, and sometimes the size of the DNA fragments is from about 100 bp to 500 bp. The conversion can be effected by a number of different methods, such as DNase I or RNase digestion, random shearing or partial restriction enzyme digestion. For discussions of protocols for the isolation, manipulation, enzymatic digestion, and the like of nucleic acids, see, for example, Sambrook et al. and Ausubel, both supra. The concentration of nucleic acid fragments of a particular length and sequence is often less than 0.1% or 1% by weight of the total nucleic acid. The number of different specific nucleic acid fragments in the mixture is usually at least about 100, 500 or 1000.

The mixed population of nucleic acid fragments are converted to at least partially single-stranded form using a variety of techniques, including, for example, heating, chemical denaturation, use of DNA binding proteins, and the like. Conversion can be effected by heating to about 80° C. to 100° C., more preferably from 90° C. to 96° C., to form single-stranded nucleic acid fragments and then reannealing. Conversion can also be effected by treatment with single-stranded DNA binding protein (see Wold, Annu. Rev. Biochem. 66:61-92 (1997)) or recA protein (see, e.g., Kiianitsa, Proc. Natl. Acad. Sci. USA 94:7837-7840 (1997)). Single-stranded nucleic acid fragments having regions of sequence identity with other single-stranded nucleic acid fragments can then be reannealed by cooling to 20° C. to 75° C., and preferably from 40° C. to 65° C. Renaturation can be accelerated by the addition of polyethylene glycol (PEG), other volume-excluding reagents or salt. The salt concentration is preferably from 0 mM to 200 mM, more preferably the salt concentration is from 10 mM to 100 mM. The salt may be KCl or NaCl. The concentration of PEG is preferably from 0% to 20%, more preferably from 5% to 10%. The fragments that reanneal can be from different substrates. The annealed nucleic acid fragments are incubated in the presence of a nucleic acid polymerase, such as Taq or Klenow, and dNTP's (i.e. dATP, dCTP, dGTP and dTTP). If regions of sequence identity are large, Taq polymerase can be used with an annealing temperature of between 45-65° C. If the areas of identity are small, Klenow polymerase can be used with an annealing temperature of between 20-30° C. The polymerase can be added to the random nucleic acid fragments prior to annealing, simultaneously with annealing or after annealing.

The process of denaturation, renaturation and incubation in the presence of polymerase of overlapping fragments to generate a collection of polynucleotides containing different permutations of fragments is sometimes referred to as shuffling of the nucleic acid in vitro. This cycle is repeated for a desired number of times. Preferably the cycle is repeated from 2 to 100 times, more preferably the sequence is repeated from 10 to 40 times. The resulting nucleic acids are a family of double-stranded polynucleotides of from about 50 bp to about 100 kb, preferably from 500 bp to 50 kb. The population represents variants of the starting substrates showing substantial sequence identity thereto but also diverging at several positions. The population has many more members than the starting substrates. The population of fragments resulting from shuffling is used to transform host cells, optionally after cloning into a vector.

In one embodiment utilizing in vitro shuffling, subsequences of recombination substrates can be generated by amplifying the full-length sequences under conditions which produce a substantial fraction, typically at least 20 percent or more, of incompletely extended amplification products. Another embodiment uses random primers to prime the entire template DNA to generate less than full length amplification products. The amplification products, including the incompletely extended amplification products are denatured and subjected to at least one additional cycle of reannealing and amplification. This variation, in which at least one cycle of reannealing and amplification provides a substantial fraction of incompletely extended products, is termed “stuttering.” In the subsequent amplification round, the partially extended (less than full length) products reanneal to and prime extension on different sequence-related template species. In another embodiment, the conversion of substrates to fragments can be effected by partial PCR amplification of substrates.

In another embodiment, a mixture of fragments is spiked with one or more oligonucleotides. The oligonucleotides can be designed to include precharacterized mutations of a wildtype sequence, or sites of natural variations between individuals or species. The oligonucleotides also include sufficient sequence or structural homology flanking such mutations or variations to allow annealing with the wildtype fragments. Annealing temperatures can be adjusted depending on the length of homology.

In a further embodiment, recombination occurs in at least one cycle by template switching, such as when a DNA fragment derived from one template primes on the homologous position of a related but different template. Template switching can be induced by addition of recA (see, Kiianitsa (1997) supra), rad51 (see, Namsaraev, Mol. Cell. Biol. 17:5359-5368 (1997)), rad55 (see, Clever, EMBO J. 16:2535-2544 (1997)), rad57 (see, Sung, Genes Dev. 11:1111-1121 (1997)) or other polymerases (e.g., viral polymerases, reverse transcriptase) to the amplification mixture. Template switching can also be increased by increasing the DNA template concentration.

Another embodiment utilizes at least one cycle of amplification, which can be conducted using a collection of overlapping single-stranded DNA fragments of related sequence, and different lengths. Fragments can be prepared using a single stranded DNA phage, such as M13 (see, Wang, Biochemistry 36:9486-9492 (1997)). Each fragment can hybridize to and prime polynucleotide chain extension of a second fragment from the collection, thus forming sequence-recombined polynucleotides. In a further variation, ssDNA fragments of variable length can be generated from a single primer by Pfu, Taq, Vent, Deep Vent, UlTma DNA polymerase or other DNA polymerases on a first DNA template (see, Cline, Nucleic Acids Res. 24:3546-3551 (1996)). The single stranded DNA fragments are used as primers for a second, Kunkel-type template, consisting of a uracil-containing circular ssDNA. This results in multiple substitutions of the first template into the second. See, Levichkin, Mol. Biology 29:572-577 (1995); Jung, Gene 121:17-24 (1992).

In some embodiments of the invention, shuffled nucleic acids obtained by use of the recursive recombination methods of the invention are put into a cell and/or organism for screening. Shuffled RANK or OPG genes can be introduced into, for example, bacterial cells (including cyanobacteria), yeast cells, fungal cells, vertebrate cells, invertebrate cells or plant cells for initial screening. Bacterial species, such as E. coli, Pseudomonas sp, Bacillus. subtilis, Burkholderia cepacia, Alcaligenes, Acinetobacter, Rhodococcus Arthrobacter, Sphingomonas are examples of suitable bacterial cells into which one can insert and express shuffled RANK or OPG genes which provide for convenient shuttling to other cell types (a variety of vectors for shuttling material between these bacterial cells and eukaryotic cells are available; see, Sambrook, Ausubel and Berger, all supra). The shuffled genes can be introduced into bacterial, fungal, mammalian, insect, or yeast cells either by integration into the chromosomal DNA or as plasmids.

Although mammalian, insect and yeast systems are most preferred in the present invention, in one embodiment, shuffled genes can also be introduced into plant cells for production purposes. Thus, a transgene of interest can be modified using the recursive sequence recombination methods of the invention in vitro and reinserted into the cell for in vivolin situ selection for the new or improved RANK or OPG property, in bacteria, eukaryotic cells, or whole eukaryotic organisms.

5. In Vivo Nucleic Acid Shuffling Formats

In some embodiments of the invention, DNA substrate molecules are introduced into cells, wherein the cellular machinery directs their recombination. For example, a library of mutants is constructed and screened or selected for mutants with improved phenotypes by any of the techniques described herein. The DNA substrate molecules encoding the best candidates are recovered by any of the techniques described herein, then fragmented and used to transfect a plant host and screened or selected for improved function. If further improvement is desired, the DNA substrate molecules are recovered from the host cell, such as by PCR, and the process is repeated until a desired level of improvement is obtained. In some embodiments, the fragments are denatured and reannealed prior to transfection, coated with recombination stimulating proteins such as recA, or co-transfected with a selectable marker such as Neo^{R} to allow the positive selection for cells receiving recombined versions of the gene of interest. Methods for in vivo shuffling are described in, for example, PCT application WO 98/13487 and WO 97/20078.

The efficiency of in vivo shuffling can be enhanced by increasing the copy number of a gene of interest in the host cells. For example, the majority of bacterial cells in stationary phase cultures grown in rich media contain two, four or eight genomes. In minimal medium the cells contain one or two genomes. The number of genomes per bacterial cell thus depends on the growth rate of the cell as it enters stationary phase. This is because rapidly growing cells contain multiple replication forks, resulting in several genomes in the cells after termination. The number of genomes is strain dependent, although all strains tested have more than one chromosome in stationary phase. The number of genomes in stationary phase cells decreases with time. This appears to be due to fragmentation and degradation of entire chromosomes, similar to apoptosis in mammalian cells. This fragmentation of genomes in cells containing multiple genome copies results in massive recombination and mutagenesis. The presence of multiple genome copies in such cells results in a higher frequency of homologous recombination in these cells, both between copies of a gene in different genomes within the cell, and between a genome within the cell and a transfected fragment. The increased frequency of recombination allows one to evolve a gene more quickly to acquire optimized characteristics.

In nature, the existence of multiple genomic copies in a cell type would usually not be advantageous due to the greater nutritional requirements needed to maintain this copy number. However, artificial conditions can be devised to select for high copy number. Modified cells having recombinant genomes are grown in rich media (in which conditions, multicopy number should not be a disadvantage) and exposed to a mutagen, such as ultraviolet or gamma irradiation or a chemical mutagen, e.g., mitomycin, nitrous acid, photoactivated psoralens, alone or in combination, which induces DNA breaks amenable to repair by recombination. These conditions select for cells having multicopy number due to the greater efficiency with which mutations can be excised. Modified cells surviving exposure to mutagen are enriched for cells with multiple genome copies. If desired, selected cells can be individually analyzed for genome copy number (e.g., by quantitative hybridization with appropriate controls). For example, individual cells can be sorted using a cell sorter for those cells containing more DNA, e.g., using DNA specific fluorescent compounds or sorting for increased size using light dispersion. Some or all of the collection of cells surviving selection are tested for the presence of a gene that is optimized for the desired property.

In one embodiment, phage libraries are made and recombined in mutator strains such as cells with mutant or impaired gene products of mutS, mutT, mutH, mutL, ovrD, dcm, vsr, umuC, umuD, sbcB, recJ, etc. The impairment is achieved by genetic mutation, allelic replacement, selective inhibition by an added reagent such as a small compound or an expressed antisense RNA, or other techniques. High multiplicity of infection (MOI) libraries are used to infect the cells to increase recombination frequency.

Additional strategies for making phage libraries and or for recombining DNA from donor and recipient cells are set forth in U.S. Pat. No. 5,521,077. Additional recombination strategies for recombining plasmids in yeast are set forth in WO 97 07205.

6. Shuffling Families of RANK and OPG

For identifying homologous genes used to shuffle a family of genes, representative alignments of RANK and OPG genes can be generated from sequences retrieved from GeneBank or an associated public database.

7. Codon Modification Shuffling

Procedures for codon modification shuffling are described in detail in SHUFFLING OF CODON ALTERED GENES, Phillip A. Patten and Willem P. C. Stemmer, filed Sep. 29, 1998, U.S. Ser. No. 60/102362 and in SHUFFLING OF CODON ALTERED GENES, Phillip A. Patten and Willem P. C. Stemmer, filed Jan. 29, 1999, U.S. Ser. No. 60/117729. In brief, by synthesizing nucleic acids in which the codons encoding polypeptides are altered, it is possible to access a completely different mutational cloud upon subsequent mutation of the nucleic acid. This increases the sequence diversity of the starting nucleic acids for shuffling protocols, which alters the rate and results of forced evolution procedures. Codon modification procedures can be used to modify any nucleic acid described herein, e.g., prior to performing nucleic acid shuffling, or codon modification approaches can be used in conjunction with oligonucleotide shuffling procedures as described supra.

In these methods, a first nucleic acid sequence encoding a first polypeptide sequence is selected. A plurality of codon altered nucleic acid sequences, each of which encode the first polypeptide, or a modified or related polypeptide, is then selected (e.g., a library of codon altered nucleic acids can be selected in a biological assay which recognizes library components or activities), and the plurality of codon-altered nucleic acid sequences is recombined to produce a target codon altered nucleic acid encoding a second protein. The target codon altered nucleic acid is then screened for a detectable functional or structural property, optionally including comparison to the properties of the first polypeptide and/or related polypeptides. The goal of such screening is to identify a polypeptide that has a structural or functional property equivalent or superior to the first polypeptide or related polypeptide. A nucleic acid encoding such a polypeptide can be used in essentially any procedure desired, including introducing the target codon altered nucleic acid into a cell, vector, virus, attenuated virus (e.g., as a component of a vaccine or immunogenic composition), transgenic organism, or the like.

8. Oligonucleotide and in Silico Shuffling Formats

In addition to the formats for shuffling noted above, at least two additional related formats are useful in the practice of the present invention. The first, referred to as “in silico” shuffling utilizes computer algorithms to perform “virtual” shuffling using genetic operators in a computer. As applied to the present invention, gene sequence strings are recombined in a computer system and desirable products are made, e.g., by reassembly PCR of synthetic oligonucleotides. In silico shuffling is described in detail in Selifonov and Stemmer in “METHODS FOR MAKING CHARACTER STRINGS, POLYNUCLEOTIDES & POLYPEPTIDES HAVING DESIRED CHARACTERISTICS” filed Feb. 5, 1999, U.S. Ser. No. 60/118854. In brief, genetic operators (algorithms which represent given genetic events such as point mutations, recombination of two strands of homologous nucleic acids, etc.) are used to model recombinational or mutational events which can occur in one or more nucleic acid, e.g., by aligning nucleic acid sequence strings (using standard alignment software, or by manual inspection and alignment) and predicting recombinational outcomes. The predicted recombinational outcomes are used to produce corresponding molecules, e.g., by oligonucleotide synthesis and reassembly PCR.

The second useful format is referred to as “oligonucleotide mediated shuffling” in which oligonucleotides corresponding to a family of related homologous nucleic acids (e.g., as applied to the present invention, interspecific or allelic variants of a RANK or OPG nucleic acid) are recombined to produce selectable nucleic acids. This format is described in detail in Crameri et al. “OLIGONUCLEOTIDE MEDIATED NUCLEIC ACID RECOMBINATION” filed Feb. 5, 1999, U.S. Ser. No. 60/118,813 and Crameri et al. “OLIGONUCLEOTIDE MEDIATED NUCLEIC ACID RECOMBINATION” filed Jun. 24, 1999, U.S. Ser. No. 60/141,049. The technique can be used to recombine homologous or even non-homologous nucleic acid sequences.

One advantage of the oligonucleotide-mediated recombination is the ability to recombine homologous nucleic acids with low sequence similarity, or even non-homologous nucleic acids. In these low-homology oligonucleotide shuffling methods, one or more set of fragmented nucleic acids are recombined, e.g., with a with a set of crossover family diversity oligonucleotides. Each of these crossover oligonucleotides have a plurality of sequence diversity domains corresponding to a plurality of sequence diversity domains from homologous or non-homologous nucleic acids with low sequence similarity. The fragmented oligonucleotides, which are derived by comparison to one or more homologous or non-homologous nucleic acids, can hybridize to one or more region of the crossover oligos, facilitating recombination.

When recombining homologous nucleic acids, sets of overlapping family gene oligonucleotides (which are derived by comparison of homologous nucleic acids and synthesis of oligonucleotide fragments) are hybridized and elongated (e.g., by reassembly PCR), providing a population of recombined nucleic acids, which can be selected for a desired trait or property. Typically, the set of overlapping family genes include a plurality of oligonucleotide member types which have consensus region subsequences derived from a plurality of homologous target nucleic acids.

Typically, family gene shuffling oligonucleotides are provided by aligning homologous nucleic acid sequences to select conserved regions of sequence identity and regions of sequence diversity. A plurality of family gene shuffling oligonucleotides are synthesized (serially or in parallel) which correspond to at least one region of sequence diversity.

Sets of fragments, or subsets of fragments, used in oligonucleotide shuffling approaches can be provided by cleaving one or more homologous nucleic acids (e.g., with a DNase), or, more commonly, by synthesizing a set of oligonucleotides corresponding to a plurality of regions of at least one nucleic acid (typically oligonucleotides corresponding to a full-length nucleic acid are provided as members of a set of nucleic acid fragments). In the shuffling procedures herein, these cleavage fragments (e.g., fragments of RANK or OPG genes) can be used in conjunction with family gene shuffling oligonucleotides, e.g., in one or more recombination reaction to produce recombinant RANK or OPG nucleic acids.

9. Chimeric Shuffling Templates

In addition to the naturally occurring, mutated and synthetic oligonucleotides discussed above, polynucleotides encoding chimeric polypeptides can be used as substrates for shuffling in any of the above-described shuffling formats. Preferred chimeras have a shuffled active site or a shuffled active site region. Art-recognized methods for preparing chimeras are applicable to the methods described herein (see, for example, Shimoji et al., Biochemistry 37: 8848-8852 (1998)). In a particular embodiment, the polynucleotide encoding a chimeric polypeptide is a chimera derived from nucleic acids encoding RANK and OPG.

In a preferred embodiment, polynucleotides encoding domain chimeras with at least one cysteine-rich domain from OPG and at least one cysteine-rich domain from RANK are constructed. Both OPG and RANK comprise four cysteine-rich TNF receptor-like domains that have been shown to be responsible for binding to RANKL. In OPG, these domains are found within residues 22-194, while in RANK they are found within residues 31-211. In both cases, the ligand binding regions, which are homologous to the TNF receptor family, comprise cysteine-rich autonomous folding domains with minimal interdomain contact, only intradomain disulfide bridges, and a small center almost exclusively consisting of backbone atoms. Although all four domains are believed to be required for ligand binding of OPG and RANK (based on domain-deletion studies, Yamaguchi et al. (1998), J. Biol. Chem. 273(9):5117-23), based on structural data, domains 2 and 3 are believed to be the domains that are primarily responsible for ligand binding.

Domain chimeras may thus be constructed by, for example, replacing one or two of the four OPG domains in an OPG backbone with a RANK domain or by replacing one or two of the four RANK domains in a RANK backbone with an OPG domain. This provides for a total of 16 possible basic constructs having different combinations of OPG and RANK sequences in the four domains.

Polynucleotides encoding one or more of such domain chimeric constructs can thus be prepared and used as a template for shuffling, either with other such chimeric constructs or in any of the above-described shuffling formats, e.g. family shuffling. Alternatively, one or more individual amino acid mutations based on knowledge obtained via e.g. random mutagenesis or shuffling, typically mutations that have been found to result in improved binding to RANKL, can be performed in a domain chimera in order to e.g. obtain improved RANKL binding affinity. In FIG. 4B, the hashmarks in the sequence alignment of the TNFR-like domains of OPG and RANK indicate predicted domain boundaries. Domain chimera polynucleotide templates may be constructed by exchanging a nucleotide sequence encoding all or part of one or more of these domains in a sequence encoding an OPG or RANK polypeptide backbone with a nucleotide sequence encoding the corresponding domain or part thereof from the other polypeptide. One or more, typically one or two, nucleotide sequences encoding an entire domain may thus be exchanged, and/or one or more nucleotide sequences encoding a part or parts of one or more domains may be exchanged. In the latter case, where only a part of a domain is exchanged, it is preferable to exchange one or more nucleotide sequences that encode one or more ligand binding subsequences, in particular one or more of the predicted ligand binding subsequences of at least three amino acid residues that are underlined in FIG. 4B.

With the four predicted domains shown in FIG. 4B being termed domains 1, 2, 3 and 4, respectively, preferred domain chimeric templates encode an OPG or RANK polypeptide backbone with all or part of a single domain 1, 2, 3 or 4 being exchanged, or with all or part of two domains, e.g. domains 1 and 4 or 2 and 3, being exchanged. When only a part of a domain is exchanged, this part preferably includes all of the predicted ligand binding residue subsequences in that domain as indicated in FIG. 4B, i.e. for domain 1 residues 7-18 and 22-32, for domain 2 residues 45-73, for domain 3 residues 90-104 and 123-125, and for domain 4 residues 137-139.

It is contemplated that polynucleotides encoding such domain chimeras based on both OPG and RANK may provide optimal templates for shuffling or other mutagenesis with the aim of producing novel proteins with improved binding to RANKL. Since both OPG and RANK bind RANKL, even though the degree of sequence identity at the amino acid level between OPG and RANK is only about 32%, it is believed that polynucleotides encoding OPG/RANK domain chimeras will provide optimal possibilities for exploiting the sequence space encompassed by OPG and RANK. For example, novel proteins with improved binding to RANKL may be obtained even though such proteins may have a relatively low amino acid sequence identity compared to one or both the parent proteins, e.g. a sequence identity to OPG and/or RANK of from about 40% up to less than about 80%, such as from about 50% or 60% to less than about 70%.

In a particular embodiment, novel RANKL binding polypeptides may be developed using a process comprising three basic steps: 1) providing an optimal backbone based on OPG and/or RANK; 2) performing affinity maturation to improve binding affinity to RANKL; and 3) providing desired characteristics in terms of half-life and/or immunogenicity. It should be noted that these three steps do not necessarily have to be performed in the order given. For example, development of the polypeptides will often involve an iterative process in which individual steps may be alternated and repeated as necessary to obtain a desired result.

As indicated above, one method contemplated to be useful for producing an optimal backbone involves producing polynucleotides encoding domain chimeras with one or more domains derived from OPG and one or more domains derived from RANK. Another useful method for this purpose is crossover oligonucleotide mediated shuffling as described above.

Affinity maturation involves alteration of a parent polypeptide, e.g. a domain chimera or otherwise optimized polypeptide backbone, so as to provide desired binding characteristics to RANKL, typically a binding affinity superior to the reference polypeptide (OPG or RANK). For affinity maturation purposes, any of the mutagenesis techniques described above, or a combination of two or more such techniques, may be employed, e.g. site-directed mutagenesis, random mutagenesis or DNA shuffling. Shuffling, for example family shuffling combined with high throughput screening using e.g. FACS (Fluorescent Activated Cell Sorting) as described below, is a particularly preferred method that is well suited for producing novel proteins with desired binding characteristics. Family shuffling may e.g. be performed using polynucleotides encoding one or more domain chimeras and/or polynucleotides encoding one or more homologous polypeptides. For example, if shuffling is performed using a polynucleotide encoding a domain chimera with an hOPG backbone having one or two RANK domains introduced therein, homologous polypeptides will be those which may be defined as being homologous to hOPG, e.g. OPG from primates or other mammals. Shuffling need not be performed directly on a domain chimera, however. One may for example choose to use suitable shuffling techniques, such as family shuffling performed on homologous wild-type sequences, in order to obtain knowledge about useful mutations, e.g. mutations that result in improved binding of wild-type hOPG or hRANK to RANKL, and to apply these mutations to a domain chimeric polypeptide.

Providing desired characteristics in terms of half-life and/or immunogenicity may be obtained by any of the techniques described herein, i.e. in particular by conjugation to a non-polypeptide moiety, for example by means of PEGylation and/or in vivo glycosylation as discussed in detail above, where appropriate accompanied by amino acid residue changes in order to introduce and/or remove one or more attachment sites.

As a non-limiting example of the mutation strategy described above, the following illustrates one possible approach to providing novel RANKL binding proteins based on domain chimeras. In this approach, several different strategies may be employed in parallel or in a random order, e.g.:

1. Construction of domain chimeras (e.g. by replacing one or two of four OPG domains with a RANK domain).

2. Family shuffling of wild-type proteins for each of OPG and RANK (e.g. primates, cat, dog, mouse, rat, etc.).

3. Random mutagenesis of ligand binding non-conserved residues (i.e. not conserved between OPG and RANK).

4. Shuffling of OPG and RANK (ligand binding residues). (Cross-over oligo/doped oligonucleotide shuffling).

5. Directed PEGylation and glycosylation of selected residues of wild-type OPG and RANK.

Once at least one round of the different strategies has been performed, amino acid changes from the best candidates from strategies 2-5 above may be incorporated into the domain chimeras from strategy 1. These candidates are then assayed, and candidates having desired binding properties to RANKL are selected and, if desired, subjected to further mutation using one or more of the techniques listed above or otherwise described herein.

Expression of the RANK and/or OPG Variants

Once assembled (by shuffling, site-directed mutagenesis, synthesis or another method), the nucleotide sequence encoding the polypeptide is inserted into a recombinant vector and operably linked to control sequences necessary for expression of the RANK and/or OPG variant in the desired transformed host cell.

It should of course be understood that not all vectors and expression control sequences function equally well to express the nucleotide sequence encoding a polypeptide described herein. Neither will all hosts function equally well with the same expression system. However, one of skill in the art may make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and the ease of purification of the products coded for by the nucleotide sequence.

The recombinant vector may be an autonomously replicating vector, i.e. a vector which exists as an extrachromosomal entity, the replication of which is independent of chromosomal replication, e.g. a plasmid. Alternatively, the vector is one which, when introduced into a host cell, is integrated into the host cell genome and replicated together with the chromosome(s) into which it has been integrated.

The vector is preferably an expression vector in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for mammalian eukaryotic hosts include, for example, vectors comprising expression control sequences from SV40, bovine papilloma virus, adenovirus and cytomegalovirus. Specific vectors are, e.g., pCDNA3.1(+)\Hyg (Invitrogen, Carlsbad, Calif., USA) and pCI-neo (Stratagene, La Jolla, Calif., USA). Useful expression vectors for yeast cells include the 2μ plasmid and derivatives thereof, the POT1 vector (U.S. Pat. No. 4,931,373), the pJSO37 vector described in Okkels, Ann. New York Acad. Sci. 782, 202-207, 1996, and pPICZ A, B or C (Invitrogen). Useful vectors for insect cells include pVL941, pBG311 (Cate et al., Cell 45, pp. 685-98 (1986)), pBluebac 4.5 and pMelbac (both available from Invitrogen). Useful expression vectors for bacterial hosts include known bacterial plasmids, such as plasmids from E. coli, including pBR322, pET3a and pET12a (both from Novagen Inc., WI, USA), wider host range plasmids, such as RP4, phage DNAs, e.g., the numerous derivatives of phage lambda, e.g., NM989, and other DNA phages, such as M13 and filamentous single stranded DNA phages.

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, Mol. Cell. Biol. 2, pp. 1304-19 (1982)) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g. a gene whose product complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For Saccharomyces cerevisiae, selectable markers include ura3 and leu2. For filamentous fungi, selectable markers include amdS, pyrG, arcB, niaD and sC.

The term “control sequences” is defined herein to include all components which are necessary or advantageous for the expression of the polypeptide of the invention. Each control sequence may be native or foreign to the nucleic acid sequence encoding the polypeptide. Such control sequences include, but are not limited to, a leader sequence, polyadenylation sequence, propeptide sequence, promoter, enhancer or upstream activating sequence, signal peptide sequence, and transcription terminator. At a minimum, the control sequences include a promoter.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g. the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol Aug. 20, 1987;196(4):947-50).

In order to improve expression in mammalian cells a synthetic intron may be inserted in the 5′ untranslated region of the nucleotide sequence encoding the polypeptide. An example of a synthetic intron is the synthetic intron from the plasmid pCI-Neo (available from Promega Corporation, WI, USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence. Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4c promoter, and the inducible GAL promoter. Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator. Examples of suitable control sequences for use in bacterial host cells include promoters of the lac system, the trp system, the TAC or TRC system, and the major promoter regions of phage lambda.

The presence or absence of a signal peptide will, e.g., depend on the expression host cell used for the production of the polypeptide to be expressed (whether it is an intracellular or extracellular polypeptide) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the Lepidopteran manduca sexta adipokinetic hormone precursor, (cf. U.S. Pat. No. 5,023,328), the honeybee melittin (Invitrogen), ecdysteroid UDPglucosyl-transferase (egt) (Murphy et al., Protein Expression and Purification 4, 349-357 (1993) or human pancreatic lipase (hpl) (Methods in Enzymology 284, pp. 262-272, 1997). A preferred signal peptide for use in mammalian cells is that of the murine Ig kappa light chain signal peptide (Coloma, M (1992) J. Imm. Methods 152:89-104), or the native OPG or RANK signal peptides. For use in yeast cells suitable signal peptides have been found to be the α-factor signal peptide from S. cereviciae (cf. U.S. Pat. No. 4,870,008), a modified carboxypeptidase signal peptide (cf. L. A. Valls et al., Cell 48, 1987, pp. 887-897), the yeast BAR1 signal peptide (cf. WO 87/02670), the yeast aspartic protease 3 (YAP3) signal peptide (cf. M. Egel-Mitani et al., Yeast 6, 1990, pp. 127-137), and the synthetic leader sequence TA57 (WO98/32867). For use in E. coli cells a suitable signal peptide have been found to be the signal peptide of ompA (EP581821).

The nucleotide sequences of the invention encoding a RANK and/or OPG variant, whether prepared by shuffling, site-directed mutagenesis, synthesis, PCR or other methods, may optionally also include a nucleotide sequence that encodes a signal peptide. The signal peptide is present when the polypeptide is to be secreted from the cells in which it is expressed. Such signal peptide, if present, should be one recognized by the cell chosen for expression of the polypeptide. The signal peptide may be homologous (e.g. be that normally associated with RANK or OPG) or heterologous (i.e. originating from another source) to the polypeptide or may be homologous or heterologous to the host cell, i.e. be a signal peptide normally expressed from the host cell or one which is not normally expressed from the host cell. Accordingly, the signal peptide may be prokaryotic, e.g. derived from a bacterium such as E. coli, or eukaryotic, e.g. derived from a mammalian, or insect or yeast cell.

Any suitable host may be used to produce the polypeptide subunits of the invention, including bacteria, fungi (including yeasts), plant, insect, mammal, or other appropriate animal cells or cell lines, as well as transgenic animals or plants. Examples of bacterial host cells include gram-positive bacteria such as strains of Bacillus, e.g. B. brevis or B. subtilis, or Streptomyces, or gram-negative bacteria, such as Pseudomonas or strains of E. coli. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278). Examples of suitable filamentous fungal host cells include strains of Aspergillus, e.g. A. oryzae, A. niger, or A. nidulans, Fusarium or Trichoderma. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Examples of suitable yeast host cells include strains of Saccharomyces, e.g. S. cerevisiae, Schizosaccharomyces, Klyveromyces, Pichia, such as P. pastoris or P. methanolica, Hansenula, such as H. polymorpha or Yarrowia. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; Hinnen et al., 1978, PNAS USA 75: 1920: and as disclosed by Clontech Laboratories, Inc, Palo Alto, Calif., USA (in the product protocol for the Yeastmaker™ Yeast Transformation System Kit). Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein may be performed as described by Invitrogen. Examples of suitable mammalian host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Additional suitable cell lines are known in the art and available from public depositories such as the American Type Culture Collection, USA. Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g. described by Ausbel et al. (eds.), 1996, Current Protocols in Molecular Biology, John Wiley & Sons, NY, USA. The cultivation of mammalian cells are conducted according to established methods, e.g. as disclosed in (Animal Cell Biotechnology, Methods and Protocols, Edited by Nigel Jenkins, 1999, Human Press Inc, Totowa, N.J., USA and Harrison M A and Rae I F, General Techniques of Cell Culture, Cambridge University Press 1997).

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g. in catalogues of the American Type Culture Collection). If the polypeptide is secreted into the nutrient medium, it can be recovered directly from the medium. If the polypeptide is not secreted, it can be recovered from cell lysates.

The resulting polypeptide may be recovered by methods known in the art. For example, it may be recovered from the nutrient medium by conventional procedures including, but not limited to, centrifugation, filtration, extraction, spray drying, evaporation, or precipitation.

The polypeptides may be purified by a variety of procedures known in the art including, but not limited to, chromatography (e.g. ion exchange, affinity, hydrophobic, chromatofocusing, and size exclusion), electrophoretic procedures (e.g. preparative isoelectric focusing), differential solubility (e.g. ammonium sulfate precipitation), SDS-PAGE, or extraction (see e.g. Protein Purification, J.-C. Janson and Lars Ryden, editors, VCH Publishers, New York, 1989).

Pharmaceutical Composition of the Invention and Its Use

In one aspect the polypeptide, the conjugate or the pharmaceutical composition according to the invention is used for the manufacture of a medicament for treatment of bone diseases or other diseases associated with the interactions of RANK, OPG and RANKL.

In another aspect the polypeptide, the conjugate or the pharmaceutical composition according to the invention is used in a method of treating a mammal, in particular a human, suffering from or at risk of suffering from osteoporosis or other bone diseases, the method comprising administering to the mammal in need thereof such polypeptide, conjugate or pharmaceutical composition.

The dose to be administered will depend on the circumstances, including the patient to be treated, the nature and cause of the condition, the nature of the RANK and/or OPG variant, the administration schedule, and whether the polypeptide or conjugate or composition is administered alone or in conjunction with other therapeutic agents.

The polypeptide or conjugate of the invention is normally administered in a composition including one or more pharmaceutically acceptable carriers or excipients. “Pharmaceutically acceptable” means a carrier or excipient that does not cause any untoward effects in patients to whom it is administered. Such pharmaceutically acceptable carriers and excipients are well known in the art, and the polypeptide or conjugate of the invention can be formulated into pharmaceutical compositions by well-known methods (see e.g. Remington's Pharmaceutical Sciences, 18th edition, A. R. Gennaro, Ed., Mack Publishing Company (1990); Pharmaceutical Formulation Development of Peptides and Proteins, S. Frokjaer and L. Hovgaard, Eds., Taylor & Francis (2000); and Handbook of Pharmaceutical Excipients, 3rd edition, A. Kibbe, Ed., Pharmaceutical Press (2000)). Pharmaceutically acceptable excipients that may be used in compositions comprising the polypeptide or conjugate of the invention include, for example, buffering agents, stabilizing agents, preservatives, isotonifiers, non-ionic surfactants or detergents (“wetting agents”), antioxidants, bulking agents or fillers, chelating agents and cosolvents.

The pharmaceutical composition of the polypeptide or conjugate of the invention may be formulated in a variety of forms, including liquids, e.g. ready-to-use solutions or suspensions, gels, lyophilized, or any other suitable form, e.g. powder or crystals suitable for preparing a solution. The preferred form will depend upon the particular indication being treated and will be apparent to one of skill in the art.

The pharmaceutical composition containing the polypeptide or conjugate of the invention may be administered intravenously, intramuscularly, intraperitoneally, intradermally, subcutaneously, sublingualy, buccally, intranasally, transdermally, by inhalation, or in any other acceptable manner, e.g. using PowderJect® or ProLease® technology or a pen injection system. The preferred mode of administration will depend upon the particular indication being treated and will be apparent to one of skill in the art. In particular, it is advantageous that the composition be administered subcutaneously, since this allows the patient to conduct the administration herself.

The pharmaceutical composition of the invention may be administered in conjunction with other therapeutic agents. These agents may be incorporated as part of the same pharmaceutical composition or may be administered separately from the polypeptide or conjugate of the invention, either concurrently or in accordance with any other acceptable treatment schedule. In addition, the polypeptide, conjugate or pharmaceutical composition of the invention may be used as an adjunct to other therapies.

All references cited herein are hereby incorporated by reference in their entirety for all purposes.

The present invention will be further illustrated by the following non-limiting examples.

### EXAMPLES

Structure Analysis Methods

Sequence Alignments

Determination of Surface Exposed Residues and Residues Involved in Ligand Binding When No Three-Dimensional Structure is Available:

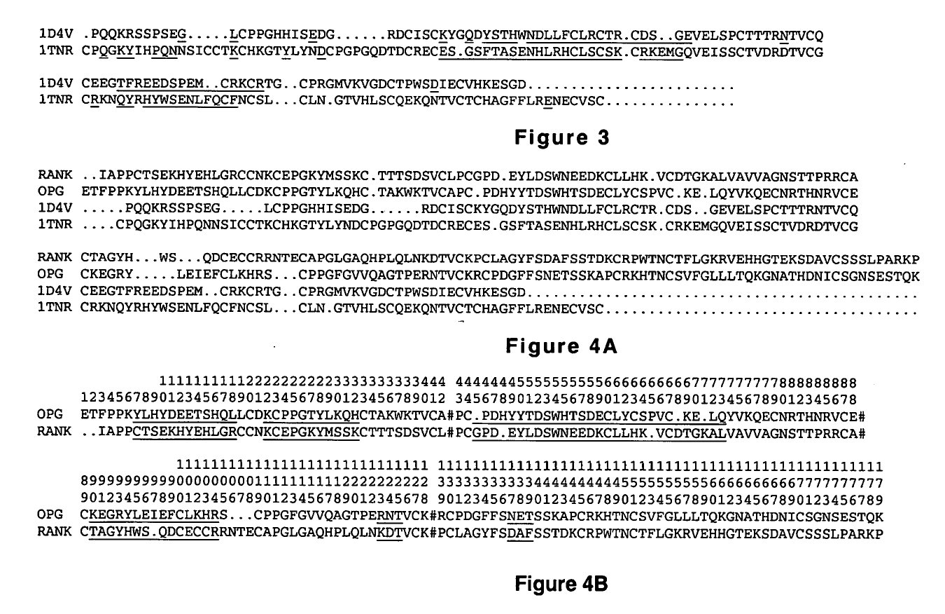

No three dimensional structure is currently known for RANK or OPG. However, it has been determined that the ligand binding domains of both RANK and OPG are members of the TNF-receptor superfamily of structures (for information on the ligand binding domains of RANK, see Anderson, et al. (1997) Nature 390, 175-9, and FIG. 1; for information on the ligand binding domains of OPG, see Simonet, et al., (1997) Cell 89, 309-19, and FIG. 2). Therefore, the sequences of human RANK and OPG may be aligned to a predefined structure-based sequence alignment of the receptors from known structures. The two structures used for this alignment with RANK and OPG are that of the extracellular domain of Death Receptor 5 and its ligand TNF-Related Apoptosis Inducing Ligand (TRAIL), and that of human tumor necrosis factor-beta and the extracellular domain of its receptor tumor necrosis factor receptor P55. These molecules were chosen as representatives of the TNF receptor structure family. The structure-based sequence alignment is performed using the program Modeler 98 (available from Molecular Simulations, Inc.), the sequence alignment being performed using the “profile/structure alignment” option of the program ClustalW (Thompson, J. D., Higgins, D. G. and Gibson, T. J.: CLUSTAL W: Improving the sensitivity of progressive multiple sequence alignment through sequence weighting, positions-specific gap penalties and weight matrix choice. Nucleic Acids Research, 22, pp. 4673-4680 (1994)). FIG. 3 shows the alignment of TNFR and DR5, and FIG. 4 shows an alignment of all four polypeptides.

From this sequence alignment, residues in the sequence to be analyzed at positions equivalent to residues exposed in at least one of the other sequences are defined as being exposed. The degree of surface exposure (solvent accessibility) is taken as the largest value for the equivalent residues in the other sequences. In case the sequence to be analyzed is at an insertion (i.e. there are no equivalent residues in the other sequences), this residue is defined as being fully exposed, as it most probably is located in a loop region.

In parallel, from this sequence alignment, residues in the sequences to be analyzed at positions equivalent to residues directly involved in ligand binding in at least one of the other sequences (from known structures) are defined as being involved in ligand binding.

Structures

Experimental three-dimensional structures of human tumor necrosis factor-beta and the extracellular domain of its receptor tumor necrosis factor receptor P55 determined by X-ray crystallography have been reported by Banner et. al (1993) Cell 73:431-45. The paper reports the structure determined at 2.85 Å resolution. The atom coordinates for the structure are deposited in the Protein Data Bank as entry 1TNR. The parts from residue Cys15 to Cys153 are detected in the structure solution. The regions outside this are thought to be too flexible to be detected at this resolution.

Experimental three-dimensional structures of human TNF-Related Apoptosis Inducing Ligand and the extracellular part of its receptor Death Receptor 5 determined by X-ray crystallography have been reported by Mongkolsapaya et. al (1999) Nat.Struct.Biol. 6 1048-53. The paper reports the structure determined at 2.2 Å resolution. The atom coordinates for the structure are deposited in the Protein Data Bank as entry 1D4V. The parts from Pro69 to Asp185 of the receptor are detected in the structure solution. The regions outside this are thought to be too flexible to be detected at this resolution.

Structure Analysis Methods

Accessible Surface Area (ASA)

The computer program Access (B. Lee and F. M. Richards, J. Mol. Biol. 55: 379-400 (1971)) version 2 (©1983 Yale University) are used to compute the accessible surface area (ASA) of the individual atoms in the structure. This method typically uses a probe-size of 1.4 Å and defines the Accessible Surface Area (ASA) as the area formed by the center of the probe. Prior to this calculation all water molecules and all hydrogen atoms should be removed from the coordinate set, as should other atoms not directly related to the protein.

Fractional ASA of Side Chain

The fractional ASA of the side chain atoms is computed by division of the sum of the ASA of the atoms in the side chain by a value representing the ASA of the side chain atoms of that residue type in an extended ALA-x-ALA tripeptide. See Hubbard, Campbell & Thornton (1991) J. Mol. Biol. 220, 507-530. For this example the CA atom is regarded as a part of the side chain of glycine residues but not for the remaining residues. The following values are used as standard 100% ASA for the side chain:

| | | | | | |
|---|---|---|---|---|---|
| Ala | 69.23 | Å² | Leu | 140.76 | Å² |
| Arg | 200.35 | Å² | Lys | 162.50 | Å² |
| Asn | 106.25 | Å² | Met | 156.08 | Å² |
| Asp | 102.06 | Å² | Phe | 163.90 | Å² |
| Cys | 96.69 | Å² | Pro | 119.65 | Å² |
| Gln | 140.58 | Å² | Ser | 78.16 | Å² |
| Glu | 134.61 | Å² | Thr | 101.67 | Å² |
| Gly | 32.28 | Å² | Trp | 210.89 | Å² |
| His | 147.00 | Å² | Tyr | 176.61 | Å² |
| Ile | 137.91 | Å² | Val | 114.14 | Å² |

Residues not detected in the structure or residues defined as being disordered are typically defined as having 100% exposure as they are thought to reside in flexible regions. In the case where an ensemble of NMR structures is analysed, the average ASA value of the ensemble is used.

Determining Distances Between Atoms:

The distance between atoms is most easily determined using molecular graphics software, e.g. InsightII® 98.0 from Molecular Simulations, Inc.

Superpositioning of Molecular Structures:

Three dimensional superimpositioning of molecular structures may also be performed using the program InsightII v. 98.0.

### Example 1

### Receptor-Ligand Interaction Analysis

Death Receptor 5

The receptor and ligand parts of the 1D4V structure were used for this example. The structure is used to assess the receptor-ligand interactions in this complex by measuring the distances between atoms of the two molecules. In addition, the receptor part of the structure is used to calculate surface accessibility of individual side chains in the molecule. In this analysis, the ligand molecules are removed before calculation.

Surface Exposure:

Performing fractional ASA calculations on the extracellular part of Death Receptor 5 of the structure resulted in the determination that the following residues have more than 25% of their side chain exposed to the surface: P69, Q70, Q71, K72, R73, S74, S75, S77, E78, G79, L80, P82, P83, H85, E89, D90, G91, R92, D93, I95, S96, K98, Y99, G100, Q101, D102, T105, H106, W107, D109, L110, L111, F112, L114, R115, T117, R118, D120, S121, G122, V124, E125, L126, P128, T130, T131, T132, R133, N134, V136, Q138, E140, E141, G142, T143, R145, E146, E147, D148, P150, E151, M152, R154, K155, R157, T158, G159, P161, R162, G163, V165, K166, V167, G168, D169, C170, T171, W173, S174, E177, V179, K181, E182, S183, G184 and D185.

The following residues were determined to have more than 50% of their side chain exposed to the surface: P69, Q70, Q71, K72, R73, S75, S77, E78, G79, E89, D90, G91, R92, D93, I95, S96, K98, Y99, G100, T105, H106, W107, D109, L111, F112, L114, R115, T117, R118, D120, S121, G122, E125, L126, P128, T132, R133, E140, E141, G142, E146, E147, D148, P150, E151, M152, R154, K155, T158, G159, R162, G163, V165, K166, V167, D169, W173, E177, K181, E182, G184 and D185.

Receptor-Ligand Interactions

Performing distance measurements between the amino acid residues of the extracellular part of Death Receptor 5 and the ligand TRAIL in the structure enabled a definition of ligand binding residues of the receptor: Gly79, Leu80, Glu89, Lys98, Gln101, Tyr103, Ser104, Thr105, His106, Asn108, Asp109, Leu110, Leu111, Phe112, Cys113, Leu114, Arg115, Cys116, Thr117, Arg118, Cys119, Asp120, Ser121, Glu123, Asn134, Thr143, Phe144, Arg145, Glu146, Glu147, Asp148, Ser149, Pro150, Glu151, Met152, Cys153, Arg154, Lys155, Cys156, Arg157 and Asp175.

TNF Receptor

The receptor and ligand part of the 1TNR structure were used for this example. The structure is used to assess the receptor-ligand interactions in this complex by measuring the distances between atoms of the two molecules. In addition, the receptor part of the structure is used to calculate surface accessibility of individual side chains in the molecule. In this analysis the ligand molecule is removed before calculation.

Surface Exposure:

Performing fractional ASA calculations on the receptor molecule of the structure resulted in the determination that the following residues have more than 25% of their side chain exposed to the surface: C15, P16, Q17, G18, K19, I21, P23, Q24, N25, N26, S27, T31, K32, H34, K35, Y40, N41, P44, G45, P46, G47, Q48, D49, D51, R53, E54, E56, S57, G58, S59, A62, S63, E64, H66, L67, R68, H69, L71, S72, S74, K75, R77, K78, E79, G81, V83, E84, I85, S86, S87, T89, V90, D91, R92, D93, V95, R99, K100, N101, H105, Y106, W107, S108, E109, N110, L111, Q113, F115, N116, S118, L119, L121, N122, T124, V125, H126, L127, S128, Q130, E131, K132, Q133, V136, T138, H140, A141, G142, F143, F144, L145, R146, E147, N148, E149, V151, S152 and C153.

The following residues were determined to have more than 50% of their side chain exposed to the surface: P16, Q17, G18, K19, I21, Q24, N25, N26, S27, T31, H34, K35, Y40, N41, P46, G47, Q48, D49, R53, E54, E56, S57, G58, A62, S63, E64, L67, R68, H69, L71, S72, S74, K75, R77, K78, E79, G81, E84, I85, S87, T89, R92, R99, K100, N101, W107, S108, E109, N110, N116, L119, L121, N122, T124, V125, H126, L127, S128, Q130, E131, K132, Q133, T138, H140, A141, G142, F144, R146, E147, E149, V151 and S152.

Receptor-Ligand Interactions

Performing distance measurements between the amino acid residues of the receptor and the ligand molecules in the 1D4V structure enabled a definition of receptor residues in close proximity to the ligand, and thus defined as being involved in ligand binding: Gln17, Lys19, Tyr20, Pro23, Gln24, Asn25, Lys32, Tyr38, Asp42, Glu56, Ser57, Gly58, Ser59, Phe60, Thr61, Ala62, Ser63, Glu64, Asn65, His66, Leu67, Arg68, His69, Cys70, Leu71, Ser72, Cys73, Ser74, Lys75, Cys76, Arg77, Lys78, Glu79, Met80, Gly81, Arg99, Gln102, Tyr103, His105, Tyr106, Trp107, Ser108, Glu109, Asn110, Leu111, Phe112, Gln113, Cys114 and Phe115.

Alignment of Death Receptor 5 and TNF Receptor

The amino acid sequence of the receptor molecule of the 1TNR structure was aligned to the amino acid sequence of the receptor molecule of the 1D4V structure using the AlignX software (InforMax Inc.). After the initial alignment, the alignment was optimized manually, based on observations from the structural superimpositions performed using Modeler 98. The resulting alignment is shown in FIG. 3.

Structure-Based alignment of OPG and RANK

The amino acid residues of the ligand binding parts of OPG and RANK, respectively, were then aligned to the above alignment using AlignX. Again, the alignment was fitted with respect to known and predicted disulfide patterns (Merewether et al., (2000) Arch. Biochem. Biophys. 375, 101-10; Anderson et al. (1997) Nature 390, 175-9). The alignment is shown in FIG. 4.

OPG and/or RANK Ligand Binding Residues

From the alignment of FIG. 4, the amino acid residues of human OPG and/or RANK that are predicted to be involved in ligand binding are assigned. This is performed by comparing the amino acid residue in question with those amino acid residues at the same position in the alignment from Death Receptor 5 or TNF receptor. If any of these amino acid residues are involved in ligand binding, then the OPG or RANK residue in question is defined as being involved in ligand binding. This is performed for each of the amino acid residues of the aligned part of human OPG and/or RANK. If an amino acid residue does not align to any residues in the Death Receptor 5 or TNF receptor amino acid sequences, then this residue is scored as the adjacent residues are scored.

It should be emphasized that this procedure in theory results in an assignment of ligand binding characteristics to any residue at a specific position in the alignment. Thus, after mutagenesis of the OPG and/or RANK molecule, any altered amino acid at any position in the alignment can be assigned the same characteristics as the original amino acid at the same position, and thus the new molecule (i.e. shuffled or otherwise mutated) can be subjected to the same mutational analyses as the original molecule.

For human OPG, the following residues are predicted by this method to be directly involved in ligand binding: Tyr28, His30, Tyr31, Glu34, Thr35, Ser36, His 37, Lys43, Tyr49, Gln52, His53, Pro66, Asp67, His68, Tyr69, Tyr70, Thr71, Asp72, Ser73, Trp74, His75, Thr76, Ser77, Asp78, Glu79, Cys80, Leu81, Tyr82, Cys83, Ser84, Pro85, Val86, Cys87, Lys88, Glu89, Leu90, Gln91, Lys108, Arg111, Tyr112, Leu113, Glu114, Ile115, Glu116, Phe117, Cys118, Leu119, Lys120, His121, Arg122, Asn139 and Glu153.

For human RANK, the following residues are predicted to be directly involved in ligand binding: Cys34, Ser36, Glu37, Tyr40, Glu41, His42, Leu43, Lys49, Tyr55, Ser58, Lys59, Gly72, Pro73, Asp74, Glu75, Tyr76, Leu77, Asp78, Ser79, Trp80, Asn81, Glu82, Glu83, Asp84, Lys85, Cys86, Leu87, Leu88, His89, Lys90, Val91, Cys92, Asp93, Thr94, Gly95, Lys96, Ala97, Leu98, Thr115, Tyr118, His119, Trp120, Ser121. Gln122, Asp123, Cys124, Glu125, Cys126, Cys127, Arg128, Asp148 and Asp161.

Due to the putative flexibility of the structures, the sidechains of the amino acid residues, and the expected uncertainty in the alignments performed, the ligand binding areas are expanded, and thus additionally, the following amino acid residues of OPG are defined as being involved in ligand binding: Leu29, Asp32, Glu33, Gln38, Leu39, Leu40, Cys44, Pro45, Pro46, Gly47, Thr48, Leu50, Lys51, Glu109, Gly110, Arg122, Arg138, Thr140, Asn152 and Thr154. Therefore, also these residues of RANK are defined as being involved in ligand binding: Thr35, Lys38, His39, Gly44, Arg45, Cys50, Glu51, Pro52, Gly53, Lys54, Met56, Ser57, Cys60, Ala116, Gly117, Lys147, Thr149, Ala162 and Phe163.

It should be noted that some of the amino acid residues adjacent to those mentioned above will be involved in positioning the amino acids that are directly binding the ligand. It may therefore sometimes be desirable to shuffle these adjacent amino acid residues together with amino acid residues directly involved in binding.

OPG Amino Acid Residue Solvent Accessibility

From the same alignment, the amino acid residues of human OPG that are predicted to be solvent accessible are assigned. This is performed by comparing the amino acid residue in question with those amino acid residues at the same position in the alignment from Death Receptor 5 or TNF receptor. If any of these amino acid residues are solvent accessible (surface exposed), then the aligned residue in question is also defined as being solvent accessible. The degree of solvent accessibility is defined as exemplified here: Asp32 of OPG aligns to a serine residue of the Death Receptor 5 in 1D4V that is calculated to be more than 25% solvent accessible but less than 50% solvent accessible. The same residue Asp32 of the OPG sequence aligns to an isoleucine residue of TNF receptor in 1TNR that is calculated to be more than 50% solvent accessible. Thus, the side chain of Asp32 is defined as being more than 50% solvent accessible. This comparison is performed for each of the amino acid residues of the aligned part of human OPG. If the amino acid residue does not align to any residues in the Death Receptor 5 or TNF receptor amino acid sequences, then this residue is scored as the adjacent residues are scored. If the amino acid residue extends beyond the borders of the Death Receptor and TNF receptor sequences in the alignment, then the residue is defined as being more than 50% solvent accessible.

As explained above, this procedure can be used to assign solvent accessibility characteristics to any residue at a specific position in the alignment, so that any altered amino acid of a mutagenized OPG or RANK sequence can be assigned the same characteristics as the original amino acid at the corresponding position in the original molecule.

For human OPG, the following amino acid residues are predicted have side chains that are more than 25% solvent accessible: Gln21, Glu22, Thr23, Phe24, Pro25, Pro26, Lys27, Tyr28, Leu29, His30, Tyr31, Asp32, Glu33, Glu34, Thr35, Ser36, His37, Gln38, Asp42, Lys43, Pro45, Pro46, Thr48, Lys51, Gln52, His53, Cys54, Thr55, Ala56, Lys57, Trp58, Lys59, Thr60, Val61, Ala63, Pro64, Pro66, Asp67, His68, Tyr69, Asp72, Ser73, Trp74, Thr76, Ser77, Asp78, Glu79, Leu81, Tyr82, Ser84, Pro85, Val86, Lys88, Glu89, Leu90, Tyr92, Val93, Lys94, Gln95, Glu96, Asn98, Arg99, Thr100, His101, Asn102, Arg103, Val104, Glu106, Lys108, Glu109, Gly110, Arg111, Leu113, Glu114, Ile115, Glu116, Phe117, Leu119, Lys120, Arg122, Ser123, Pro125, Pro126, Gly127, Gly129, Val130, Val131, Gln132, Ala133, Gly134, Thr135, Pro136, Glu137, Arg138, Val141, Lys143, Arg144, Cys145, Pro146, Asp147, Gly148, Phe149, Phe150, Ser151, Asn152, Glu153, Thr154, Ser155, Ser156, Lys157, Ala158, Pro159, Cys160, Arg161, Lys162, His163, Thr164, Asn165, Cys166, Ser167, Val168, Phe169, Gly170, Leu171, Leu172, Leu173, Thr174, Gln175, Lys176, Gly177, Asn178, Ala179, Thr180, His181, Asp182, Asn183, Ile184, Cys185, Ser186, Gly187, Asn188, Ser189, Glu190, Ser191, Thr192, Gln193, Lys194, Cys195, Gly196, Ile197, Asp198, Val199, Thr200 and Leu201.

For human OPG, the following amino acid residues are predicted have side chains that are more than 50% solvent accessible: Gln21, Glu22, Thr23, Phe24, Pro25, Pro26, Lys27, Tyr28, Leu29, His30, Tyr31, Asp32, Glu33, Thr35, Ser36, His37, Gln38, Asp42, Pro45, Pro46, Lys51, Gln52, His53, Cys54, Thr55, Ala56, Lys57, Trp58, Lys59, Thr60, Val61, Ala63, Pro64, Pro66, Asp67, His68, Asp72, Ser73, Trp74, Ser77, Asp78, Glu79, Leu81, Tyr82, Ser84, Pro85, Val86, Lys88, Glu89, Leu90, Val93, Lys94, Glu96, Asn98, Thr100, His101, Lys108, Glu109, Gly100, Leu113, Glu114, Ile115, Glu116, Leu119, Lys120, Ser123, Pro125, Pro126, Gly127, Gly129, Val130, Val131, Gln132, Ala133, Thr135, Pro136, Glu137, Arg138, Val141, Lys143, Arg144, Cys145, Pro146, Asp147, Gly148, Phe149, Ser151, Glu153, Thr154, Ser155, Ser156, Lys157, Ala158, Pro159, Cys160, Arg161, Lys162, His163, Thr164, Asn165, Cys166, Ser167, Val168, Phe169, Gly170, Leu171, Leu172, Leu173, Thr174, Gln175, Lys176, Gly177, Asn178, Ala179, Thr180, His181, Asp182, Asn183, Ile184, Cys185, Ser186, Gly187, Asn188, Ser189, Glu190, Ser191, Thr192, Gln193, Lys194, Cys195, Gly196, Ile197, Asp198, Val199, Thr200 and Leu201.

RANK Amino Acid Residue Solvent Accessibility

From the same alignment, the amino acid residues of human RANK that are predicted to be solvent accessible are assigned. This is performed by comparing the amino acid residue in question with those amino acid residues at the same position in the alignment from Death Receptor 5 or TNF receptor. If any of these amino acid residues are solvent accessible, then the residue in question is defined as being solvent accessible. The degree of solvent accessibility is defined as exemplified here: Tyr40 of RANK aligns to a Pro residue of Death Receptor 5 in 1D4V that is calculated to be less than 25% solvent accessible. The same residue Tyr40 of the RANK sequence aligns to an Pro residue of TNF receptor in 1TNR that is calculated to be more than 25% solvent accessible. Thus, the side chain of Tyr40 is defined as being more than 25% solvent accessible. This comparison is performed for each of the amino acid residues of the aligned part of human RANK. If the amino acid residue does not align to any residues in the Death Receptor 5 or TNF receptor amino acid sequences, then this residue is scored as the adjacent residues are scored. If the amino acid residue does extend beyond the borders of the Death Receptor and TNF receptor sequences in the alignment, then the residue is defined as being more than 50% solvent accessible.

For human RANK, the following amino acid residues are predicted have side chains of which more than 25% are solvent accessible: Ile30, Ala31, Pro32, Pro33, Cys34, Thr35, Ser36, Glu37, Lys38, His39, Tyr40, Glu41, His42, Leu43, Gly44, Arg45, Asn48, Lys49, Glu51, Pro52, Lys54, Ser57, Ser58, Lys59, Thr61, Thr62, Thr63, Ser64, Asp65, Ser66, Val67, Leu69, Pro70, Gly72, Pro73, Asp74, Glu75, Asp78, Ser79, Trp80, Glu82, Glu83, Asp84, Lys85, Leu87, Leu88, Lys90, Val91, Asp93, Thr94, Gly95, Lys96, Ala97, Val99, Ala100, Val101, Val102, Ala103, Asn105, Ser106, Thr107, Thr108, Pro109, Arg100, Arg111, Ala113, Thr115, Ala116, Gly117, Tyr118, Trp120, Ser121. Gln122, Asp123, Glu125, Cys126, Arg128, Arg129, Asn130, Thr131, Glu132, Ala134, Pro135, Gly136, Gly138, Ala139, Gln140, His141, Pro142, Leu143, Gln144, Leu145, Asn146, Lys147, Val150, Lys152, Pro153, Leu155, Ala156, Gly157, Tyr158, Phe159, Ser160, Asp161, Ala162, Phe163, Ser164, Ser165, Thr166, Asp167, Lys168, Cys169, Arg170, Pro171, Trp172, Thr173, Asn174, Cys175, Thr176, Phe177, Leu178, Gly179, Lys180, Arg181, Val182, Glu183, His184, His185, Gly186, Thr187, Glu188, Lys189, Ser190, Asp191, Ala192, Val193, Cys194, Ser195, Ser196, Ser197, Leu198, Pro199, Ala200, Arg201, Lys202, Pro203, Pro204, Asn205, Glu206, Pro207, His208, Val209, Tyr210, Leu211, Pro212 and Leu213.

For human RANK, the following amino acid residues are predicted have side chains of which more than 50% are solvent accessible: Ile30, Ala31, Pro32, Cys34, Thr35, Ser36, Glu37, Lys38, His39, Glu41, His42, Leu43, Gly44, Arg45, Asn48, Glu51, Pro52, Ser57, Ser58, Lys59, Thr62, Thr63, Ser64, Asp65, Ser66, Val67, Leu69, Pro70, Gly72, Pro73, Asp74, Asp78, Ser79, Trp80, Glu83, Asp84, Lys85, Leu87, Leu88, Lys90, Val91, Asp93, Thr94, Gly95, Lys96, Ala97, Ala100, Val101, Ala103, Asn105, Thr107, Thr108, Thr115, Ala116, Gly117, Trp120, Ser121. Glu125, Cys126, Arg128, Arg129, Asn130, Thr131, Glu132, Ala134, Pro135, Gly136, Gly138, Ala139, Gln140, His141, Pro142, Gln144, Leu145, Asn146, Lys147, Val150, Lys152, Pro153, Leu155, Ala156, Gly157, Tyr158, Ser160, Asp161, Phe163, Ser164, Ser165, Thr166, Asp167, Lys168, Cys169, Arg170, Pro171, Trp172, Thr173, Asn174, Cys175, Thr176, Phe177, Leu178, Gly179, Lys180, Arg181, Val182, Glu183, His184, His185, Gly186, Thr187, Glu188, Lys189, Ser190, Asp191, Ala192, Val193, Cys194, Ser195, Ser196, Ser197, Leu198, Pro199, Ala200, Arg201, Lys202, Pro203, Pro204, Asn205, Glu206, Pro207, His208, Val209, Tyr210, Leu211, Pro212 and Leu213.

### Example 2

### Selection of Mutation Sites

Site Directed Mutagenesis

Mutagenesis of the Candidate Molecules

It should be emphasized that all discussed and prioritized site-directed mutations in the text below originate from observations performed on the ligand binding domain of native human OPG and/or the ligand binding domain of native human RANK. One or more of these suggested site-directed mutations in the native molecules may also be introduced into chimeric molecules produced by shuffling (MolecularBreeding™) of OPG and/or RANK. This is possible due to the fact that such shuffled molecules will comprise parts originating from each of the native molecules (OPG and/or RANK), and because the amino acid sequences of the products of the MolecularBreeding™ reactions will comprise alternating pieces from the native molecules in the same linear order as defined by the above-discussed alignment.

Lysines:

Substitution of Lysines to Remove Attachment Points for PEGylation:

The effect of PEGylation of the OPG and RANK proteins will be evaluated by using site directed PEGylation. The lysine residues in both proteins (OPG: Lys27, Lys43, Lys51, Lys57, Lys59, Lys88, Lys94, Lys108, Lys120, Lys143, Lys162, Lys176 and Lys194; RANK: Lys38, Lys49, Lys54, Lys59, Lys85, Lys90, Lys147, Lys168, Lys180, Lys189 and Lys202) will be mutated to arginine in order to evaluate the effect of PEGylation of the different lysine residues. The lysine residues that are predicted to be in close proximity to RANKL (OPG: Lys43, Lys51, Lys88, Lys108 and Lys120; RANK: Lys38, Lys49, Lys54, Lys85, Lys90, Lys96 and Lys147) will preferably be mutated to arginine in order to maintain an effective binding to RANKL. The lysine residues will be mutated to arginine residues either individually or several together in different combinations to determine the effect of PEGylation at the various positions.

Substitution to Lysines to Introduce Attachment Points:

Substitutions of surface exposed residues to lysine residues will introduce new attachment points for PEGylation. Therefore, if additional PEGylation is desired, existing residues predicted to be solvent exposed (see above) will be replaced by lysine residues. Preferred residues for substitution to lysines are arginine residues. Preferably, the lysine residues will be inserted by substitution at one or more of the following positions: In OPG: Arg99, Arg103, Arg144 and Arg161; and in RANK: Arg110, Arg111, Arg129, Arg170, Arg181 and Arg201. Alternatively, a lysine may be inserted as a replacement for one or more of the five N-terminal amino acid residues or one or more of the five C-terminal amino acid residues in either one of the sequences.

Glycosylation Sites.

Introduction of New N-glycosylation Sites

Additional sites for in vivo N-glycosylation may be inserted in areas that do not interfere with ligand binding, instead of amino acid residues that are not in close proximity to a Pro residue, and at stretches that are predicted to be solvent accessible.

Sites with the sequence pattern N-X-S/T/C-Z (where N is asparagine, X is any amino acid residue except proline, S/T/C is either serine, threonine or cysteine, preferably serine or threonine, and most preferably threonine, and Z is any amino acid residue which may be identical to or different from X and which preferably is different from proline) are potential sites for in vivo glycosylation. New glycosylation sites can therefore be introduced by substitution of preferably one or two residues that introduces the above mentioned sequence pattern. Sites where the residue to be an “N” is more than 25% side chain exposed and “X” and “Z” are not P, and where none of the residues to be substituted are a Cys residue involved in a disulphide bond, are preferable. More preferable are positions already having an N or an S/T in the “position 1” or “position 3”, respectively, of the above mentioned sequence pattern, so that a glycosylation site may be introduced by substitution of a single amino acid residue. Even more preferable are positions already having an S/T in the “position 3”. Still more preferably, the side chain ASA has more than 50% surface exposure. In all instances, it is preferable not to introduce N-glycosylation sites at positions defined as being part of the ligand binding interface, although residues having more than 25% and in particular more than 50% side chain ASA in the complex can still often be targets for introduction of a new N-glycosylation site without seriously altering the ligand binding.

It should be noted that although the asparagine residue of the N-glycosylation site is where the oligosaccharide moiety is attached during glycosylation, such attachment cannot be achieved unless the other amino acid residues of the N-glycosylation site are present. Accordingly, the term “N-glycosylation site” as used in connection with such amino acid residue modifications is understood as referring the sequence pattern N-X-S/T/C-Z defined above.

Application of these rules results in the following positions having more than 25% side chain ASA being targets for introduction of a new N-glycosylation site. For the sake of simplicity, only the residue which is “N” or which is to be modified to “N” is listed below. It will be clear, however, that the entire sequence pattern N-X-S/T/C-Z must be present in order to introduce a new glycosylation site.

Additional N-glycosylation sites will thus preferably be inserted in the OPG polypeptide at Thr55, Ala56, Lys57, Trp58, Lys59, Tyr92, Val93, Lys94, His101, Asn102, Val104, Gly129, Val130, Val131, Gln132, Val141, Gly148, Phe149, Ser155, Arg161, Lys162, Val168, Phe169, Gly170, Leu171, Leu172, Leu173, Thr174, Gln175, His181, Asp182, Ile184, Ser186, Gly187, Asn188, Ser189, Glu190, Ser191, Thr192, Lys194, Gly196, Ile197, Asp198 and Val199.

Preferably, additional N-glycosylation sites will be inserted in the RANK polypeptide at Thr61, Thr62, Thr63, Ser64, Asp65, Val99, Ala100, Val101, Val102, Ala103, Gly104, Ala113, Arg129, Asn130, Gly138, Ala139, Gln144, Leu155, Ala156, Gly157, Tyr158, Phe159, Ser164, Ser165, Thr166, Phe177, Leu178, Gly179, Lys180, Arg181, Val182, Glu183, His184, His185, Gly186, Thr187, Glu188, Lys189, Ser190, Asp191, Val193 and Ser195.

Removal of Potential N-glycosylation Sites:

If the nature or distribution of the carbohydrate groups attached to any of the molecules produced as described herein are different from the natural forms of glycosylation for these molecules, it might be advantageous to remove one or more of the putative N-glycosylation sites by mutagenesis. The removal of a potential N-glycosylation site can be performed by ensuring that a potential glycosylation site with the sequence pattern N-X-S/T/C-Z as defined above is altered so that this sequence pattern is no longer present. This may, for example, be performed by replacing an asparagine residue in such a sequence by another hydrophilic amino acid, for instance a glutamine, threonine or serine residue. The putative N-glycosylation sites that might be changed in OPG are: Asn98, 152, 165, and 178. The putative N-glycosylation sites that might be changed in RANK are Asn105 and 174. Further, if potential N-glycosylation sites are created in a modified polypeptide according to the invention, by shuffling or otherwise, such N-glycosylation sites may if desired be removed in the same manner.

Cysteines

Removal/Insertion of Cysteine Amino Acid Residues:

One or more of the cysteines that do not align between the two sequences (OPG: Cys83 and 97; RANK: Cys34, 46, 126, and 127) may if desired be removed by substitution with any small amino acid residue, i.e. Ala, Val, Gly or possibly Ser. Alternatively, cysteine residues can be substituted with the corresponding amino acid from the other sequence (OPG Cys83 to His, and Cys97 to Gly; RANK: Cys34 to Tyr, Cys46 to Leu, Cys126 to Lys, and Cys127 to His). These substitutions might be necessary in order to obtain shuffled proteins that will fold with the correct disulfide bond pattern, without having unpaired cysteine residues which might give problems when purifying the variant proteins.

### Example 3

### Mutagenesis

Example of RANK or OPG Family Shuffling

RANK or OPG genes are cloned from various primate and mammalian species, e.g. mouse, rat, dog, cat, sheep, goat, cow, horse, rabbit, hamster, guinea pig, humans, chimpanzee, gorilla, orangutan, baboon, mandrill, monkey, bonobo, marmoset, macaque, lemur, gibbon, shrew, siamang, and/or tamarin. The diversity found in the these RANK or OPG genes is used for synthetic family shuffling as described in “Oligonucleotide mediated nucleic acid recombination” by Crameri et al., filed Sep. 28, 1999 (U.S. Ser. No. 09/408,392) and “Oligonucleotide mediated nucleic acid recombination” by Crameri et al., filed Jan. 18, 2000 (PCT/U.S. Ser. No. 01203) using assembly of oligonucleotides encoding the diversity. After the synthetic family shuffling, the resulting PCR fragment is isolated and digested with KpnI and XhoI and ligated into the same restriction enzyme sites of the pYHANKb or pYhRANKbE yeast display expression vectors (Sequence 1, 2). The ligation mixture is transformed into E. coli and a small fraction is plated on LB-Amp agar plates and 10 to 20 randomly picked E. coli colonies are DNA sequenced in the OPG or RANK encoding region to estimate the shuffling frequency in the libraries. The rest of the transformation mixture is grown up in 20 ml of LB-Amp and plasmid is prepared from the transformed E. coli. Libraries with an average of 2 to 5, 4 to 7, or 5 to 10 amino acid exchanges per individual clone compared to the human wt sequence are transformed into the S. cerevisiae strain EBY 100 (Invitrogen, CA, USA) and displayed on yeast as described in the pYD1 yeast display manual (Invitrogen) and screened using the FACS procedure as described below.

Example of Shuffling of RANK and OPG

In the structural alignment of OPG and RANK proteins (shown in FIG. 4), underlined amino acid residues indicate that this amino acid residue is predicted to be in close proximity to the ligand. These six areas are shuffled by making oligonucleotides that contain the RANK nucleotide sequence and doped with nucleotides that encode the OPG amino acid residues in these areas. An example of such a doped oligonucleotide covering the second region (amino acid residues YMSSK in RANK) is:

5′-GTGTGAACCTGGTAAATAC(90% Tri-ATG/10% Tri-CTG) (90% Tri-TCT/10% Tri-AAA) (90% Tri-TCT/10% Tri-CAG) (90% Tri-AAA, 10% Tri-CAT) TGTACTACCACTAGTGACAG-3′

Tri- followed by 3 letters in capital means a trinucleotide encoding the respective codon. The trinucleotides are synthesized and coupled as described by Kayushin et al., Nucleic Acids Research, 24, 3748-3755, 1996.

Similarly oligonucleotides are synthesized covering the five other areas indicated in FIG. 4. The nucleotide sequence encoding the soluble part of RANK is isolated as a 550 bp fragment by PCR. This PCR fragment is DNase treated as described in Stemmer (1994) “DNA shuffling by random fragmentation and reassembly: In vitro recombination for molecular evolution.” Proc. Natl. Acad. Sci. USA 91:10747-10751. 50 to 100 bp fragments from the DNase treatment are isolated using agarose gel electrophoresis and purification. 0.5 to 1 pmol of these fragments are mixed with 1 pmol, 3 pmol, 6 pmol or 12 pmol of the doped oligonucleotides described above. The mixtures are used in a PCR reaction and approximately one tenth of the PCR product is used in a new PCR reaction with primers PR17: 5′-ACGATAAGGTACCAATCGCT-3′ and PR20: 5′-AATCGAGACCGAGGAGAGGG-3′ added amplifying the 550 bp nucleotide sequence containing the RANK/OPG shuffled nucleotide sequences. The resulting PCR product is isolated and digested with KpnI and XhoI and ligated into the same restriction enzyme sites of the pYHRANKb or pYhRANKbE yeast display expression vectors (sequences 1, 2). The ligation mixture is transformed into E. coli and a small fraction is plated on LB-Amp agar plates and 10 to 20 randomly picked E. coli colonies are DNA sequenced in the 550 bp region to estimate the amino acid exchange frequency in each of the libraries made from 1 pmol, 3 pmol, 6 pmol or 12 pmol of the doped oligonucleotides as described above. The rest of the transformation mixture is grown up in 20 ml of LB-Amp and plasmid is prepared from the transformed E. coli. Libraries with 2 to 4, 3 to 6, or 4 to 10 amino acid exchanges in average are transformed into the S. cerevisiae strain EBY 100 (Invitrogen) and displayed on yeast as described in the pYD1 yeast display manual (Invitrogen) and screened using the FACS procedure as described below.

Libraries are also made without including doped oligonucleotides encoding the last region or with only one, two or three of the regions.

Alternatively, the six areas with amino acid residues predicted to be in close proximity to the ligand (FIG. 4b) are shuffled by making oligonucleotides that contain the OPG nucleotide sequence and doped with nucleotides that encode the RANK amino acid residues in these areas by the same method as described above.

### Example 4

### Expression

The cysteine-rich TNF receptor-like domains have been shown to be the parts of both OPG and RANK that are responsible for binding to RANKL. These domains can be expressed as soluble proteins in various heterologous expression systems (E. coli, baculovirus, yeast, and mammalian cells). Since it has been shown that dimeric molecules bind RANKL better than monomeric, the proteins may be expressed as dimers e.g. by fusing the variant proteins to an Fc-domain from IgG1.

The proteins may be expressed in a suitable heterologous expression system.

The libraries of variant proteins may be evaluated by testing each protein for expression level and comparing this with their ability to bind soluble RANKL (human RANKL amino acid residues 158-316).

This may be performed by expressing the cDNA libraries on the surface of cells (e.g. by using yeast display (Kieke, et al., (1997) Prot. Eng. 10, 1303-10), mammalian cells that have been fused with protoplasts (see below), or by using phage display systems) as fusion proteins with a membrane attaching part (e.g. agglutinin2 in yeast, phage membrane proteins in E. coli, or a traditional membrane spanning domain in mammalian or insect cells), and another part that is a well established epitope tag (e.g. myc-, E-, V5-, or Flag-tag). After identification of a library clone that expresses a compound that binds RANKL, the clone cDNA may be isolated and cloned into an appropriate expression vector, and subsequently expressed as a soluble protein in a heterologuos expression system. The compound may also be expressed as a chimeric fusion protein, i.e. as a fusion protein with a Fc region of IgG1 C-terminal of the RANKL binding region.

Expression of RANKL

The soluble part of RANKL is used to evaluate the quality of the variant proteins produced according to the invention.

The cDNA encoding the soluble RANKL has been cloned by PCR from pORF5-hTRANCE v.21 (Invivogen), and inserted into different expression vectors.

For production in Sf9 cells using the baculovirus system, the cDNA (amino acid residues 158-316) has been inserted into the pVL1392 vector in frame with the signal sequence of human OPG (Willard, et al., (2000) Protein Expr.Purif. 20 48-57) (Sequence 3). The expressed molecule will be purified from the medium of infected Sf9 cells using published procedures.

For production in Sacharomyces cerevisiae or Pichia pastoris, the cDNA encoding amino acid residues 158-316 has been inserted into yeast expression vectors (pJSo37, pPic-ZalphaA) downstream and in frame with the cDNA encoding Sacharomyces cerevisiae alpha mating factor signal sequence, a KEX2 cleavage site and a Flag-tag (Sequence 4).

The expressed molecule will be purified from the medium of transformed cells using standard chromatographic methods, including affinity purification on a column with immobilized monoclonal antibody recognizing the Flag-tag, and/or using affinity purification on a Protein G sepharose column with immobilized OPG-Fc or RANK-Fc chimeric proteins.

For production in E. coli, the cDNA encoding amino acid residues 158-316 has been inserted in frame and downstream of the cDNA encoding a Flag-tag into pSE380 a bacterial expression vector (Sequence 5).

The expressed molecule will be purified from the cytoplasm of transformed E. coli cells. Alternatively, the protein may be purified from solubilized inclusion bodies, refolded using dilution into a renaturing buffer or alternatively, by dialysis against a renaturing buffer. The chromatographic steps may include an affinity purification using a anti-Flag column (Sigma).

The purified soluble RANKL can be characterized using standard procedures.

A vector encoding full length RANKL may be constructed by using the constructs above and inserting the remaining part of the RANKL encoding cDNA. The insert encoding human RANKL amino acid residues 1-317 may be inserted into a mammalian expression vector. Transfected cells expressing the membrane bound RANKL molecule as detected by immunological methods may be used in the mutein/RANKL functional competition assay discussed below.

Expression of OPG and OPG-Related Molecules

Codon Optimisation of the RANKL Binding Part of OPG

The sequence encoding the TNF receptor-like domain of OPG (amino acid residues 22-194) has been codon optimized using the data from http://www.kazusa.or.jp/codon/. First, the preferred yeast codons were chosen (a randomly mixed choice of the most common codon for each amino acid, and the second most common codon encoding that amino acid, if applicable and above 5% of the codons for that amino acid in S. cerevisiae). Secondly, the resulting cDNA was adapted to the preferred human codons by removing the rare codons and replacing those with a more common human codon if this codon was not below 3% in yeast. This sequence is Sequence 6.

Expression of the RANKL Binding Part of OPG in Mammalian Cells.

The codon optimized cDNA was synthesized using synthetic oligonucleotides, and verified by DNA sequencing of both strands. The cDNA was restriction digested and ligated together with an Fc-encoding cDNA into an expression vector pcDNA3.1hyg by DNA ligation. The resulting construct (Sequence 7) encodes amino acid residues 1-194 of human OPG, a Leu-Glu dipeptide and amino acid residues 247-475 of human IgG1 (AAA02914) with a Cys to Ser substitution at position 249. The chimeric protein will be expressed in stable and transiently transfected mammalian cells.

Expression of Full-Length OPG in Mammalian Cells.

A cDNA encoding OPG with codons optimized for human codons will be synthesized using synthetic oligonucleotides. The cDNA will be inserted into a mammalian expression vector. The protein will be expressed from transiently transfected and stable cells, and the protein will be purified using standard procedures.

Expression of RANK and RANK-Related Molecules

Codon Optimisation of RANK

The sequence encoding the TNF receptor-like domain of RANK (amino acid residues 22-194) has been codon optimized using the data from http://www.kazusa.or.jp/codon/. First, the preferred yeast codons were chosen (a randomly mixed choice of the most common codon for each amino acid, and the second most common codon encoding that amino acid, if applicable and above 5% of the codons for that amino acid in S. cerevisiae). Secondly, the resulting cDNA was adapted to the preferred human codons by removing the rare codons and replacing those with a more common human codon if this codon was not rare in yeast. Sequence 8.

Expression of the RANKL Binding Part of RANK in Mammalian Cells.

The codon optimized cDNA was synthesized using synthetic oligonucleotides, and verified by DNA sequencing of both strands. The cDNA was restriction digested and ligated together with an Fc-encoding cDNA into an expression vector pcDNA3.1hyg by DNA ligation. The resulting construct (Sequence 9) encodes amino acid residues 1-213 of human RANK, a Leu-Glu dipeptide and amino acid residues 247-475 of human IgG1 (AAA02914) with a Cys to Ser substitution at position 249. The chimeric protein will be expressed in stable and transiently transfected mammalian cells.

Expression of Full Length RANK in Mammalian Cells.

The cDNA encoding RANK will be synthesized using synthetic oligonucleotides. The cDNA will be ligated into a pcDNA3.1hyg vector (Invitrogen) and the resulting vector DNA will be used in the mutein/RANKL functional competition assay detailed below.

Expression and Initial Screening of Shuffled Molecule Libraries

Expression of Shuffled Molecule Libraries on the Surface of Yeast Cells

Diversity libraries will be generated in pYD1, as detailed above.

The cDNA sequences encoding the sequences that are to be shuffled will be cloned into the pYD1 expression vector (Invitrogen) downstream of and in-frame with the Aga2 ORF, a thrombin cleavage site, and upstream of and in-frame with a E-tag or V5-tag, and a hexa-histidine tag. See sequences 1, 2 and 10 for examples of the extracellular encoding human RANK, and the ligand binding part of human OPG inserted into yeast display vectors.

Transformation of the yeast cells, and expression and display of the protein libraries will be performed as detailed in the manufacturers protocol (pYD1 Yeast Display Vector Kit manual, version C, Invitrogen, catalog No. V835-01). The generated diversity libraries will be evaluated in FACS sorting assays.

Expression of Shuffled Molecule Libraries on the Surface of Mammalian Cells

Alternatively, the generated diversity libraries may be evaluated in FACS sorting assays (see below) by expressing the compounds on the surface of mammalian cells. For this purpose, the libraries may be generated in an appropriate mammalian expression vector, preferably pcDNA-derived, and expressed as fusion proteins of a C-terminal affinity tag (e.g. E-, V5-, myc-, Flag-, Fc-, or Express-tag) and a membrane spanning domain (e.g. the membrane spanning part of the RANK polypeptide (amino acid residues 214-233), the membrane spanning part of RANKL, or other suitable membrane anchoring polypeptide stretches). The constructs can also be fused in-frame to a IgG1 Fc-domain.

The diversity may be generated as detailed above, followed by transformation of the pool of diversified cDNA constructs into E. coli HB101 cells using standard protocols.

Introduction to Protoplast Fusions

Protoplast fusion is a technique that enables the expression of bacterial DNA in a eukaryotic system. The protoplast fusion protocol has two distinct manipulations: the formation of bacterial protoplasts and the fusion of bacterial and recipient cells. The cell wall of the bacteria must first be sufficiently degraded to enable fusions. The formation of these bacterial protoplasts is accomplished by exposure to lysozyme, followed by short periods of incubation. Fusions are facilitated by exposure to polyethylene glycol. An advantage of protoplast fusions, compared to SuperFect® (Qiagen) transfections, is that fusions may be carried out immediately following transformations: transfected DNA does not have to be isolated and prepared. Furthermore, the fusions are nearly clonal, allowing for the expression of single plasmid constructs. This is quite advantageous when working with libraries of significant diversity, as it allows for the assay and selection of individual sequences. However, transfection rates are significantly lower than SuperFect®—ranging from 2-15%. Optimized rates of transfection will vary depending on choice of plasmid construct. The following protocol has been optimized for a specific plasmid, and rates of transfection will vary with changes in the protocol. It is advisable to vary some parameters to come up with a specific method for each individual application.

Bacterial Protoplasts

E. coli HB101 containing the plasmids are grown at 37° C. in Luria Broth containing appropriate selection antibiotic (100 μg/ml ampicillin) to an absorbance of 0.6-0.7 at 666 nm. Chloramphenicol is added to 200 μg/ml and the culture is incubated at 37° C. for 12-16 hours to amplify plasmid copy numbers.

1. Bacteria from 25 ml of culture are pelleted by centrifugation at 3500 rpm for 15 minutes at room temperature. The culture supernate is removed by aspiration.

2. The bacterial pellet is resuspended in 1.25 ml of chilled 20% sucrose/0.05 M Tris-HCl, pH 8.

3. T₄ Lysozyme (Ready-Lyse, Epicentre) is prepared immediately before as a 5 mg/ml in 0.25 M Tris-HCl, pH 8 stock. 0.25 ml of lysozyme is added to the bacterial suspension and incubated on ice for six minutes.

4. 0.5 ml of 0.25 M EDTA, pH 8, is added to the mixture and incubated for 5 minutes on ice.

5. 0.5 ml of 0.5 M Tris-HCl, pH 8, is added to the mixture and incubated for 10 minutes in a 37° C. water bath.

6. The bacterial suspension is then diluted with 10 ml warm (37° C.) DMEM containing 10% sucrose and 10 mM MgCl₂.

7. This is incubated for 10 minutes at room temperature. Protoplasts are now ready for fusion.

Bacteria are examined with a phase-contrast microscope to determine that 99-100% of the bacteria are protoplasts. Plasmids and plasmid copy numbers are checked routinely by preparing and analyzing plasmid DNA from protoplasts used for gene transfer (Adapted from Oi, V. T. and S. L. Morrison 1986. Chimeric Antibodies. BioTechniques 4, No. 3: 219).

Protoplast Fusion

1. Mammalian cell lines (Cos) are cultured in Cos Medium (DMEM, 10% FCS, 1% Pen-Strep and Glutamine mix). Do not let cells grow greater than 80% confluent. Higher densities will greatly compromise transfection efficiencies.

2. Seed 1×10⁶ cells per T75 flask 24 hrs before fusion (should achieve ˜70-80% confluence).

3. Media overlaying the cells is removed by aspiration.

4. To remove all traces of Pen-Strep, the flasks are washed thoroughly 2×10 ml PBS.

5. Twelve ml protoplast suspension are added to each flask (˜10,000-fold excess over cells), and flasks are centrifuged at 1500 rpm for 10 min at 25° C.

6. Supernatants are carefully removed by suction.

7. Eight ml prewarmed (37° C.) 45% PEG1500 is added at room temperature, incubated for 7 min, and removed by suction.

8. Cells are washed three times using 10 ml serum-free DMEM.

9. 20 ml DMEM containing 10% FCS, 1% Pen-Strep and Glutamine mix are added to each flask. (Choice of medium may vary if working with different cell lines.) Medium is changed after 24 hrs. Protein expression may be analyzed and sorted using FACS at 24, 48, or 72 hours. (Adapted from Tan, R. and A. D. Frankel, A Novel Glutamine-RNA Interaction Identified by Screening Libraries in Mammalian Cells, PNAS in press.)

FACS Analysis and Sorting

Using the surface display system and a Fluorescent Activated Cell Sorting (FACS), system cells can be isolated based on the expression of protein on their cell surface as well as the affinity of this protein for a soluble receptor of choice. Both yeast display and protoplast fusion systems, using e.g. CHO cells, can be sorted using this system.

The RANKL binding affinity of each surface-displayed protein receptor-related molecule can be determined from equilibrium binding titration curves. Cells displaying the receptor related protein are incubated in varying concentrations of labeled soluble human RANKL. A flow cytometer (e.g. a FACSCalibur™) is used to measure the mean fluorescence of the cell populations. The equilibrium dissociation constant, Kd, can be fitted using a suitable model (e.g. nonlinear least-squares curve fit).

The surface display construct can also include E, V5 or other epitope tags to allow ligand binding to be normalized by the number of “fusions” per cell. Because surface expression varies by over an order of magnitude from cell to cell, normalization is important to avoid artifacts related to expression efficiency.

### Example 5

### Sorting and Purification

Measuring the Surface-Displayed Protein Affinity for Soluble Ligands

Aliquots of 5E5-5E6 cells are collected by centrifugation, mixed with biotinylated ligand at a range of concentrations spanning the expected K_{d} and allowed to approach equilibrium at 25° C. or 37° C. (60 min).

Cells are pelleted by centrifugation, washed in ice-cold phosphate-buffered saline with 2% fetal bovine serum (PBS/FBS) and resuspended in a dilution of streptavidin-phycoerythrin (SA-PE). The cells are incubated on ice for 30 min in the dark. Cells are again washed in PBS/FBS and resuspended in PBS/FBS to an appropriate volume for flow cytometric analysis.

The cells are examined using a Becton Dickinson FACS Calibur flow cytometer. The population is gated by light scatter to avoid consideration of clumped cells. Events from ≧10,000 events are collected. The mean fluorescence intensity of the population of cells is recorded. A nonlinear least-squares curve fit is used to determine the equilibrium dissociation constant (Kd) from the fluorescence data. A monovalent, equilibrium binding model is assumed.

R+L←→LR

Ka=1/Kd=[LR]/[L][R],

where R is the displayed receptor-related protein, L is the ligand, and LR is the complex. Then, the fraction of protein molecules that have bound ligand, Y, is given by

Y=nKa[R]/(1+Ka[R]),

where n is the fluorescence intensity when binding is completely saturated. (VanAntwerp and Wittrup, Biotechnol. Prog. 2000, 16, 31-37)

Sorting

For sorting, a total of 5E6-5E7 cells are pelleted and washed in phosphate-buffered saline with 2% fetal bovine serum (PBS/FBS). The pellet is resuspended in PBS/FBS and Anti-tag antibody (e.g. Monoclonal Mouse Anti-E-tag antibody, Amersham Pharmacia Biotech) and biotinylated ligand (e.g. RANKL) is added. The final concentration of antibody in the mixture should be above saturation (typically between 5 and 50 nM). The “binding molecule” concentration is kept below 1 nM.

The cells are incubated for at least 1 hr on ice, at 25° C. or 37° C. in the dark. After incubation, the cells are rinsed 1× with PBS/FBS and resuspended in PBS/FBS. If a non-conjugated Anti-E-tag antibody is used, a FITC-labeled secondary anti-mouse IgG antibody is added (e.g. FITC labeled Rabbit Anti-Mouse IgG). Phycoerythrin conjugated streptavidin is added in the same incubation step.

The cells are incubated for 30-60 minutes on ice in the dark. After incubation, the cells are rinsed 1× with PBS/FBS and resuspended in PBS/FBS.

Fluorescently labeled cells are sorted using a Becton Dickinson Vantage cell sorter. The instrument is gated to accept only single yeast cells (on the basis of light scatter). The fluorescence of individual cells is monitored for both phycoerythrin (PE) and fluorescein-isothiocyanate (FITC). The population of mixed cells is gated to select 0.1 to 5% of the total cells observed, collecting those cells with highest PE (ligand binding) to FITC (epitope tag) signal ratio.

Cells are either collected by bulk sampling or collected directly into 96-well plates (prefilled with 100 μl media/well).

The cDNA from the sorted cells may be used for another round of shuffling before or after further in vitro evaluation, or the cDNA or cells directly may be used for larger scale protein expression experiments as detailed below.

Expression of Protein from FACS Sorted Cells

The sorted cells are grown as individual colonies using standard procedures.

If the sorted cells are from a yeast display library, the cells may be plated on minimal dextrose medium and grown for 2 days at 30° C. Each clone is then grown in YNB-CAA medium containing 2% glucose to a OD600 between 2 and 5 as detailed in the manufacturers protocol (pYD1 Yeast Display Vector Kit manual, version C, Invitrogen, catalog No. V835-01). The cells are then transferred into YNB-CAA containing 2% galactose and grown for 48 hours at 20-25° C. At predetermined time points, aliquots are removed and analysed for protein production by using FACS or BIAcore analysis. When the recombinant protein production is optimal, the cells are pelleted and resuspended in thrombin cleavage buffer (20 mM Tris-HCl, 150 mM NaCl, 2.5 mM CaCl, pH8.4). The cleaved recombinant proteins are then purified using E-tag affinity chromatography as described by the manufacturer (Pharmacia).

Alternatively, the sorted cells may be plated on minimal dextrose and grown for 2 days at 30° C. The individual insert cDNA are then recovered using PCR with vector primers. The resulting fragment is restriction digested and cloned into a suitable expression vector with a signal sequence, and preferably a downstream in-frame cDNA encoding a dimerisation domain or site (see above). The compound is then expressed as a soluble protein using standard procedures of the.

If the sorted cells are from a protoplast fusion experiment, the cDNAs may be amplified directly from lysed cells using vector primers and standard procedures. The pool of PCR fragments is then restriction digested and cloned into a suitable expression vector with a signal sequence, and preferably a downstream in-frame cDNA encoding a dimerisation domain or site (see above). The compound is then expressed as a soluble protein using standard procedures known in the art.

Purification of the Compounds

The proteins may be purified from cell media using standard liquid chromatography procedures. Preferably, tag-affinity purification chromatography are used when applicable. Before further in vitro evaluation the proteins may be purified to at least 60% purity as judged by coomassie brillant blue stained reducing SDS-PAGE gels.

The fusion proteins may be purified using standard techniques. Specifically, the Fc-fusion proteins are purified utilizing the binding of the Fc-region to Protein G-sepharose.

### Example 6

### Expression of the RANKL Binding Part of OPG on the Surface of Yeast Cells

The codon optimised hOPG cDNA has been cloned into pYD1 downstream of and in-frame with the Aga2 ORG, and upstream of and in-frame with a V5-tag and a hexahistidine tag. The resulting construct is shown in Sequence 10

Transformation of the yeast cells, and expression and display of the protein libraries were performed as detailed in the manufacturers protocol (pYD1 Yeast Display Vector Kit manual, version C, Invitrogen, catalog No. V835-01). Selected transformants were tested for display by using FACS analysis with monoclonal anti-V5 antibody (Invitrogen), and with monoclonal anti-OPG antibody (R&D Systems). As controls, yeast cells with an empty pYD1 vector and yeast cells alone were used.

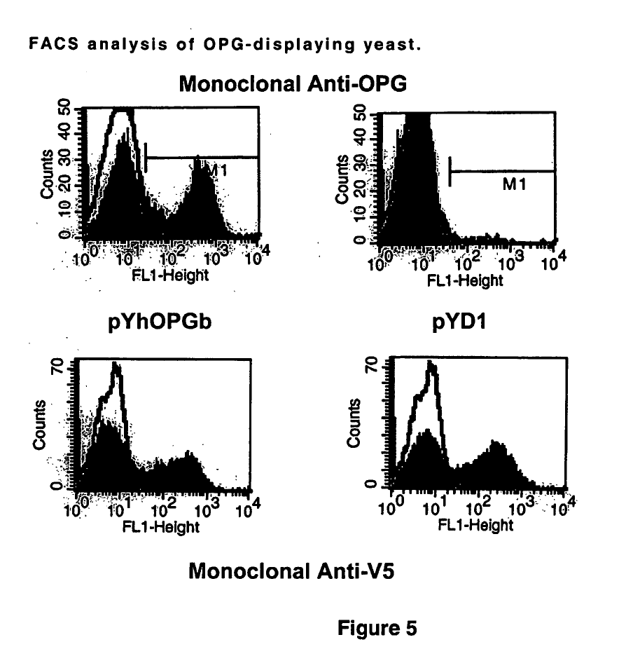

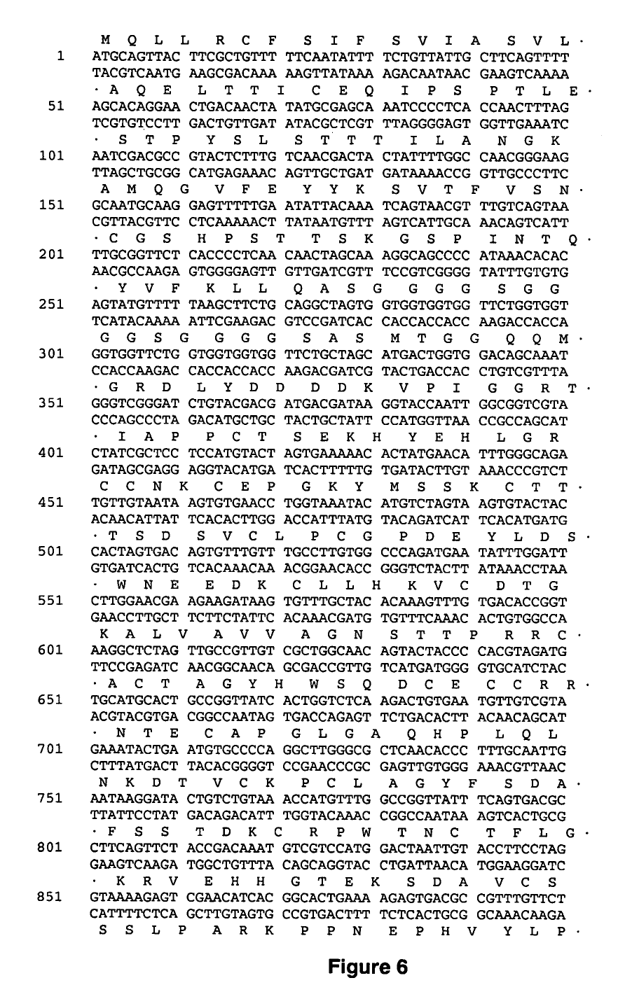

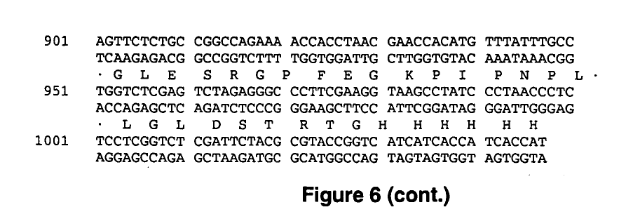

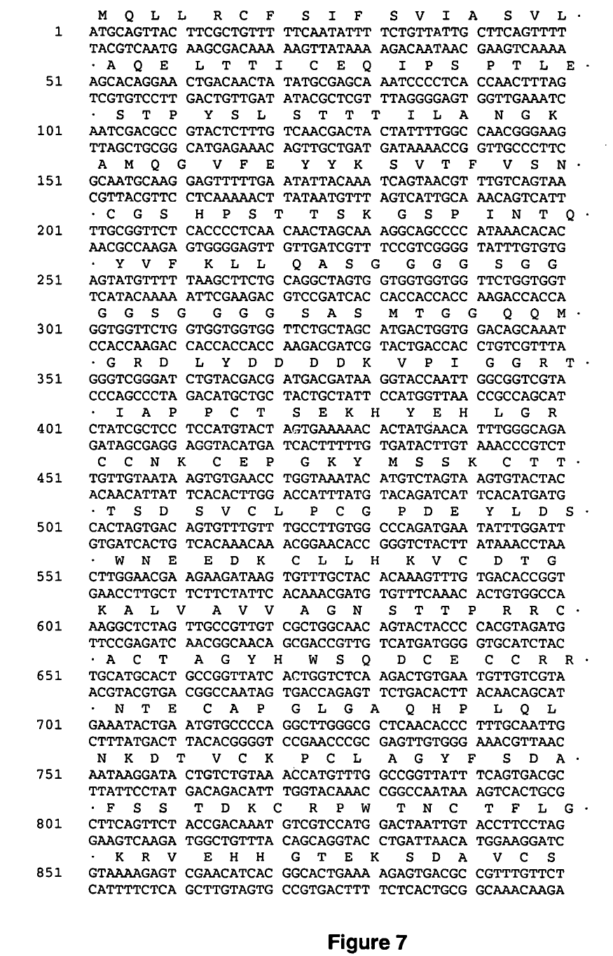

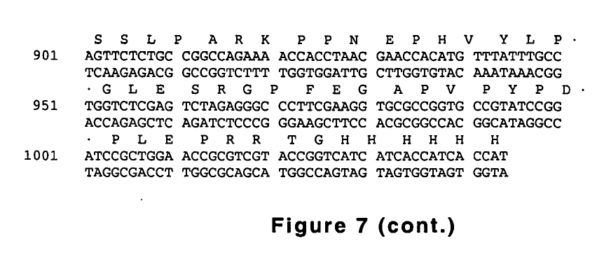

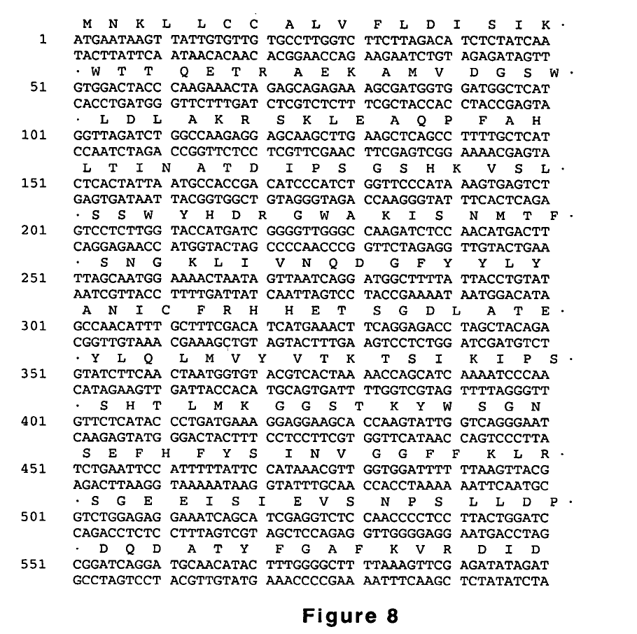

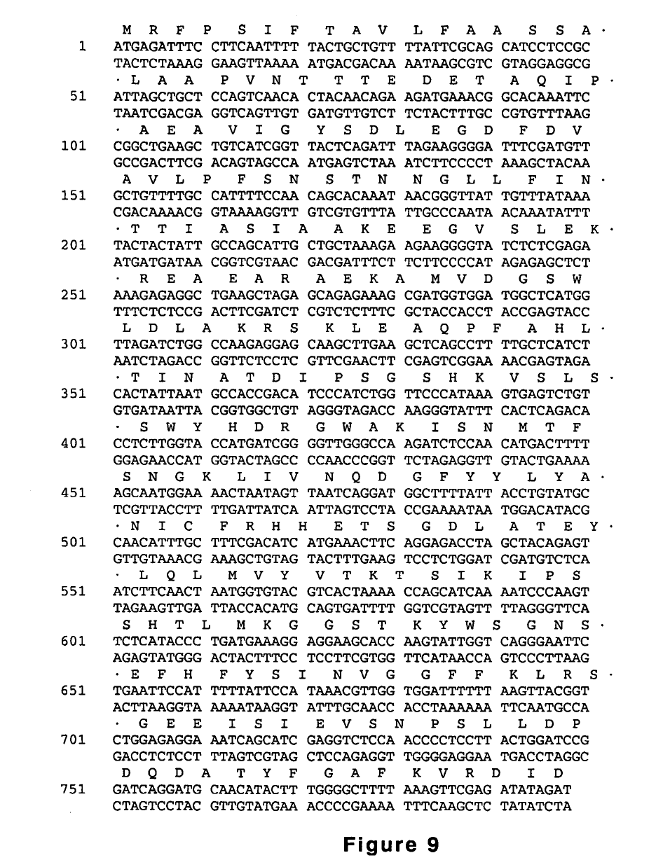

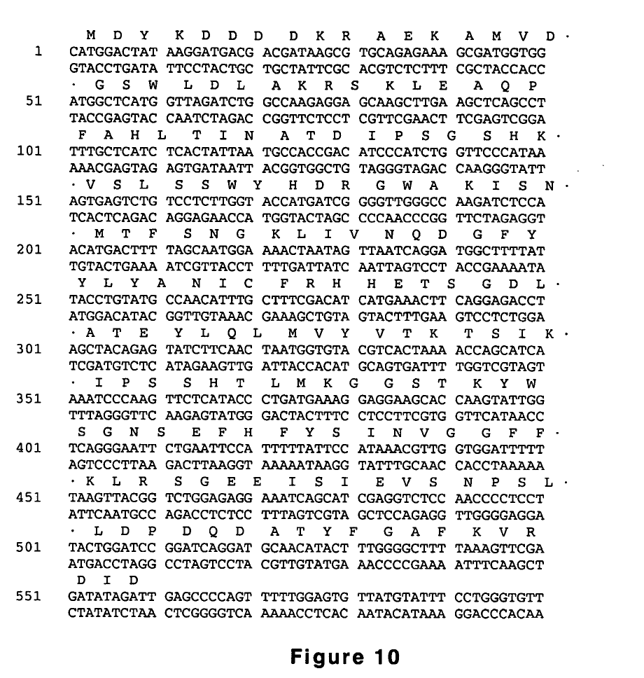

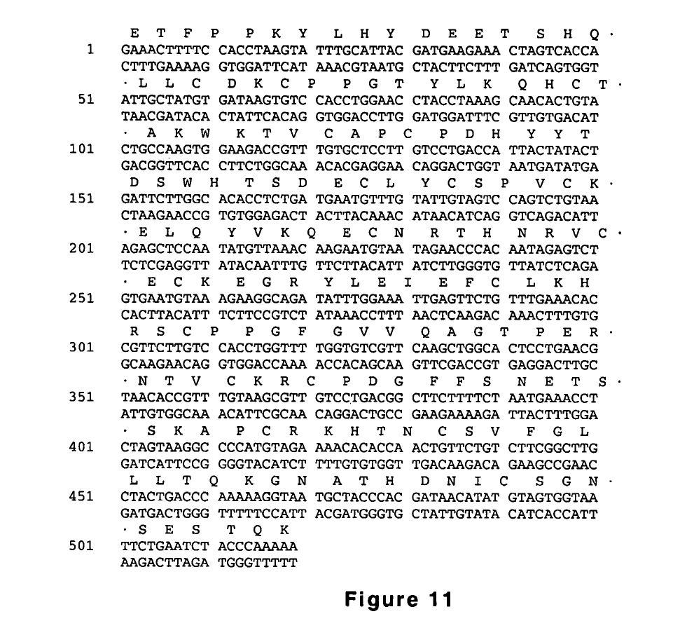

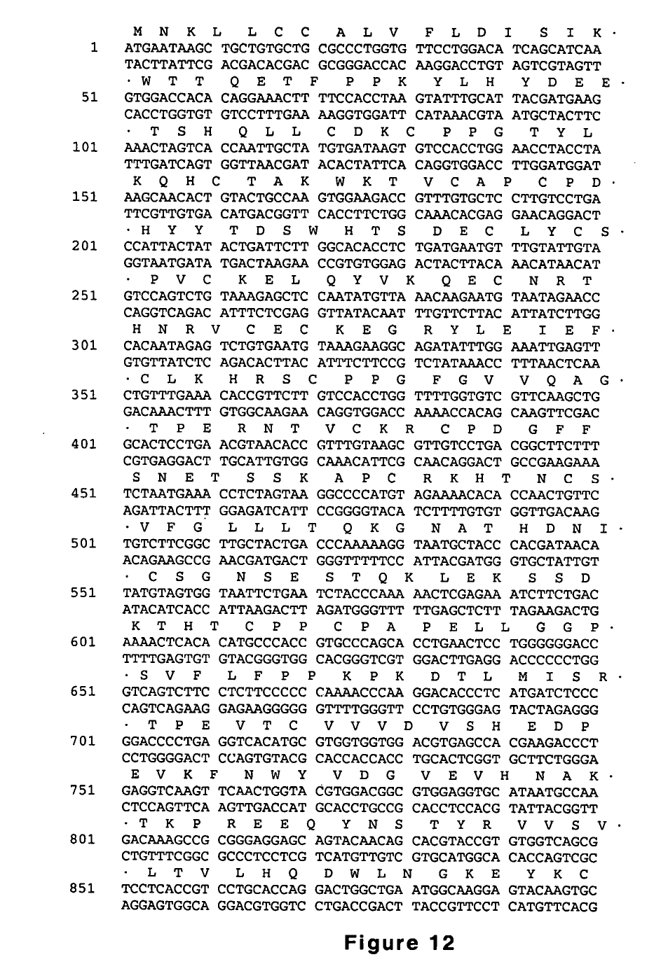

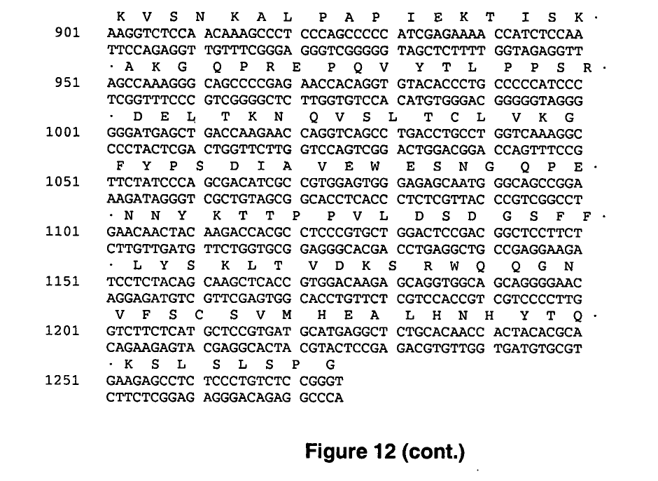

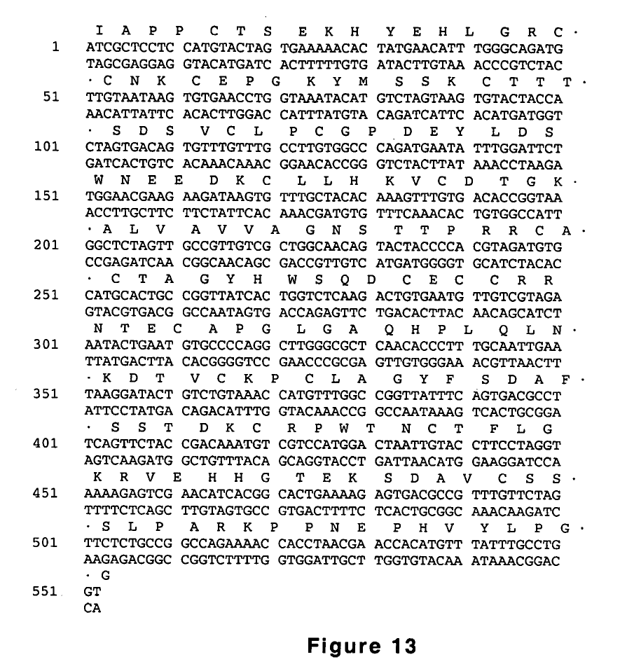

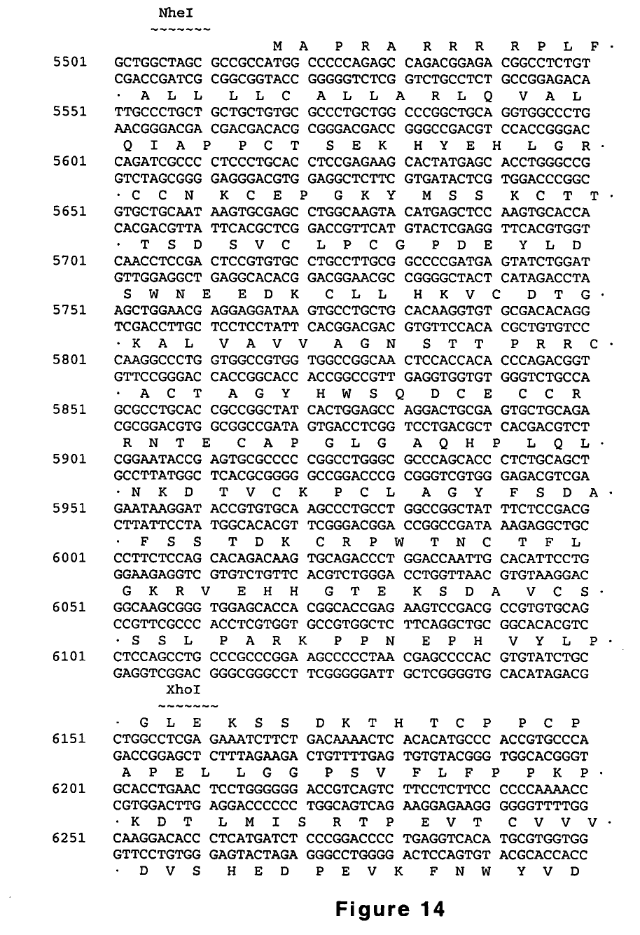

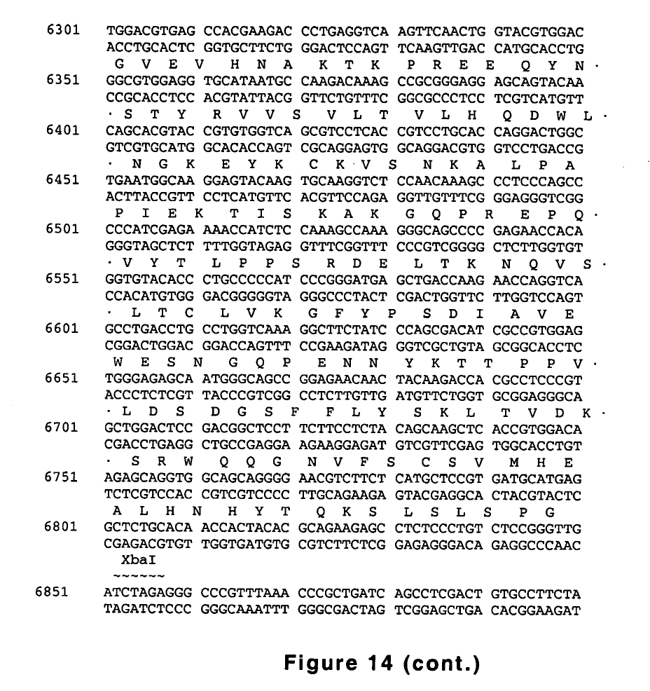

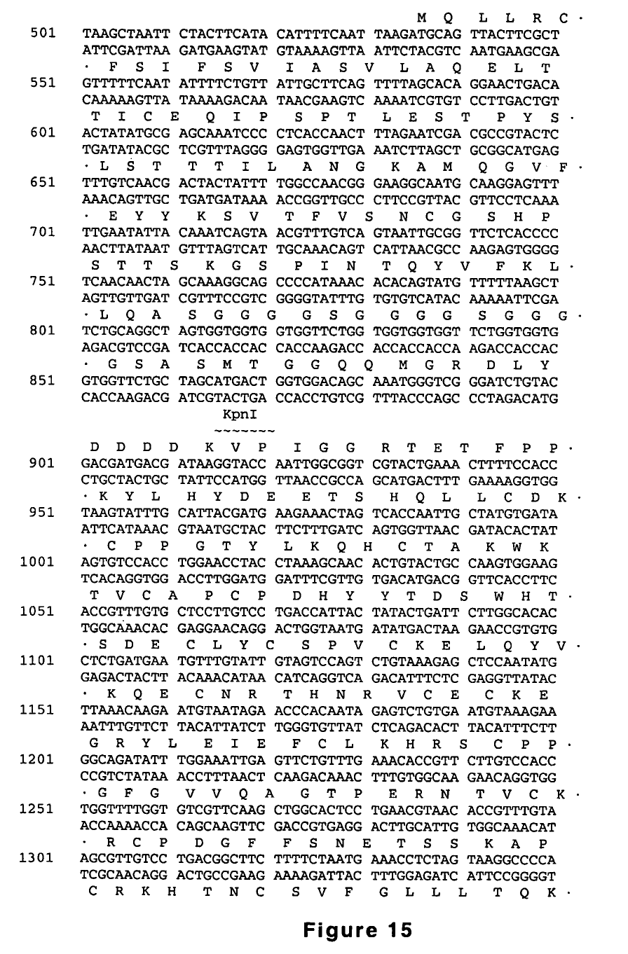

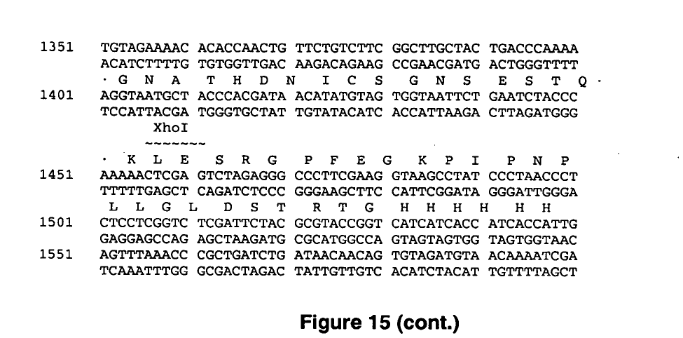

Briefly, 5 ml of cells (OD600=1) were spun down and washed in 1 ml PBS. The washed cells were spun down, and resuspended in 750 μl PBS+2.6 mg/ml BSA. The suspended cells were then divided into three aliquots. To one aliquot there was added 1 μg anti-OPG antibody, to the second aliquot was added 500 ng anti-V5 antibody. All three aliquots were incubated 30 minutes on ice. The cells were spun, washed and resuspended in 100 μl PBS+BSA (2.6 mg/ml). 5 μl RAM FITC was added to all samples, and these were subsequently incubated 30 minutes on ice. The cells were spun, washed, spun and resuspended in 5 ml PBS. The resulting FACS analysis data shown in FIG. 5 indicates that the RANKL binding part of OPG is displayed efficiently on the surface of yeast cells using this system.

By using the strategy of example 3 and the expression as outlined above a high number of OPG variants were made and evaluated according the in vitro analysis outlined under Kinetic Analysis of Shuffled variants (example 7). OPG variants with improved K_{d} in relation to wild-type hOPG were obtained, including:

T71A,K108N-hOPG(22-194),

R111W-hOPG(22-194),

K108M,R111W-hOPG(22-194),

T154L-hOPG(22-194).

For the sake of clarity hOPG(22-194) indicates the amino acid sequence of human OPG from position 22 to 194 as can be seen in FIG. 2. Thus, hOPG(22-194) has the amino acid sequence:

etfppkylhydeetshqllcdkcppgtylkqhctakwktvcapcpdhyytdswhtsdeclycspvckelqyvkqecnrthnrvcec kegryleiefclkhrscppgfgvvqagtperntvckrcpdgffsnetsskapcrkhtncsvfgllltqkgnathdnicsgnsestqk.

For instance, the indication T71A,K108N means that threonine in position 71 is exchanged with alanine and lysine in position 108 is exchanged with asparagine.

### Example 7

### In Vitro Evaluation

Secondary In Vitro Evaluation

Secondary in vitro evaluation may include RANKL binding characterisation using BIAcore analysis as detailed below and/or measurement of the compounds' ability to inhibit RANK NF-kB signalling in a RANK:RANKL competition assay as detailed below. Depending on the results from these assays the cDNA encoding the compound may be used as one of the parent molecules for shuffling in a subsequent molecular breeding experiment, and/or may be subjected to further purification and/or subjected to further site directed mutagenesis in order to mutagenize a certain residue (or several residues) and/or subjected to chemical modification and/or subjected to in vivo evaluation using procedures as detailed below.

In order for a compound to be evaluated as “successful” in terms of binding affinity to RANKL, the unmodified compound (i.e. without chemical modification such as PEGylation) should outcompete RANKL mediated RANK signaling at least as good as equimolar amounts of at least one of the relevant control proteins (human OPG (residues 21-201) and/or human RANK (residues 30-213) in the functional competition assay below.

Kinetic Analysis of Shuffled Variants

It is possible to demonstrate increased binding affinity of cell surface displayed RANK and OPG muteins by Flow Cytometer analysis. Briefly, cells expressing shuffled muteins are incubated with varying concentrations of fluorescently tagged RANKL protein. The amount of bound RANKL at equilibrium is determined by FACS analysis. Under these conditions, the concentration of RANKL that gives 50% of maximal binding is equivalent to the KD of the displayed OPG/RANK protein.

Alternatively, the binding affinity of shuffled muteins can be determined by surface plasma resonance analysis (SPR). By measuring changes in SPR, the BIAcore series of instruments measures, in real time, the interaction of a chip-conjugated and soluble protein. In this way, both Kₐ and K_{d} can be directly measured as opposed to equilibrium studies that yield only KD.

After gene shuffling, cell surfaced displayed variants will be recloned into secretory vectors and soluble versions expressed. Flowing these soluble OPG/RANK proteins over chip-coupled RANK allows measurement of Kₐ, K_{d}, and calculated KD (KD=Kₐ/K_{d}). Several variations of this experimental set-up are possible. These include: i) changes in the binding constellation with coupled, rather then soluble, variants; ii) other immobilization methods such as antibody or Nickel capture; and iii) measuring chip equilibrium affinity rather then calculating it from association and dissociation data.

Mutein/RANKL Functional Competition Assay:

Assay Outline:

It has previously been published that activation of the RANK receptor by RANKL leads to activation of NF-κB (Wong et al., PNAS 273, 28355 1998). Consequently, transcription is activated at promoters containing multiple copies of the NF-κB regulatory DNA element. It is thus possible to measure RANKL activity by use of an NF-κB luciferase reporter gene introduced into cells engineered to express or naturally expressing the RANK receptor. In the presence of a fixed concentration of RANKL, increasing concentrations of a RANKL binding protein would lead to a decrease in luciferase signal from such cells. Alternatively, a fixed concentration of a RANKL binding protein would lead to a rightward shift of a RANKL dose response curve measured as luciferase from such cells.

Mutein Screen:

HeLa cells were co-transfected with NF-κB Luc (Stratagene, CA, USA) and pcDNA 3.1/hygro (Invitrogen) and cell colonies were isolated by selection in media containing Hygromycin B. Cell clones were screened for luciferase activity in the presence or absence of TNF-α. A clone showing the highest ratio of stimulated to unstimulated luciferase activity was selected. These cells do not express the RANK receptor since no increase in luciferase signal was observed upon RANKL stimulation. The RANK receptor can be stably introduced by co-transfection of an expression plasmid encoding the receptor and a plasmid conferring G418 resistance. Clones responding to RANKL stimulation by an increase in luciferase signal can be selected. One such clone is selected for use in an assay to screen muteins for RANKL binding activity. 10,000 cells/well from this clone are seeded in 96-well white cell culture plates (Packard) in media without phenol red and incubated overnight. Muteins are added to the wells in various concentrations. Subsequently, a constant amount of RANKL, sufficient to give rise to 70-90% of maximum luciferase activity, is added to all wells and the plates are incubated for 5 hours. Plates are sealed after addition of LucLite substrate (Packard Bioscience, Groningen, The Netherlands) and luminescence is measured on a TopCount (Packard) in SPC (single photon counting) mode. Each individual plate will contain wells incubated with RANKL alone as a stimulated control and other wells containing normal media as an unstimulated control. The ratio between stimulated and unstimulated luciferase activity will serve as an internal standard for both mutein activity and experiment-to-experiment variation.

Another setup of this assay will be performed with cells expressing membrane-bound RANKL in place of soluble RANKL.

Osteoclastogenesis and Osteoclast Activity Assays

Selected compounds will be tested for their ability to inhibit RANKL-mediated osteoclastogenesis, and for their ability to inhibit RANKL-mediated osteoclast activtity. The assay procedure will be similar to that reported by Shalhoub et al., (1999) J. Cell. Biochem. 72 251-61, Fox, et al., (2000) J. Cell. Physiol. 184 334-40, and Faust, et al., (1999) J. Cell. Biochem. 72 67-80.

### Example 8

### In Vivo Evaluation

The biological activity of the compounds may be evaluated by using animal experiments (Hsu 1999, Simonet 1997, Tomoyasu, (1998) Biochem. Biophys. Res. Commun. 245 382-7).

The circulation time of the compound may be evaluated in rodents or higher animals (animals with bone remodeling: apes, pigs, dogs, etc.). Measurement of the circulating concentration may be performed by using ELISA, BIACORE® or activity analysis.

The bone degradation inhibitory effect may be evaluated in growing rodents by injecting a formulation of the compound (i.v., s.c., or intraperitonaelly) and subsequently measuring the bone mass, bone mineral density, bone fracture strength, and number of osteoclasts.

Upon administration to normal rodents, effective compounds are expected to result in an immediate lowering of circulating ionized calcium levels with maximal effect reflecting the half-life and activity of the compound.

The bone degradation inhibitory effect can also be evaluated in adult rodents with osteoporosis-like symptoms (i.e. ovariectomized rats). The osteoporotic phenotype of the animals should be partially or completely removed by effective compounds.

The bone degradation inhibitory effect can also be evaluated in adult animals with bone remodeling (e.g. dogs, pigs or apes). These animals are tested for bone fracture strength after a period of administration of the compound. In addition, the standard tests may be performed on these animals, e.g. bone mineral density, osteoclast number, calcium levels, and total bone mass.

A typical experiment may be performed using adult normal female rats that have been subjected to ovariectomy, e.g. three groups of five animals. One group is administered formulation compounds only, one group is administered OPG-Fc or RANK-Fc produced as described above, and a third group is administered the compound of interest (i.e. a protein compound isolated from a second round of shuffling that has been subjected to site directed mutagenesis to substitute a single lysine with an arginine residue, expressed in CHO cells, purified, PEGylated, and subsequently purified from excess PEG groups and non-pegylated protein). Blood can then be drawn every 2-4 hours and assayed for concentration of the administered compound, and blood ionized calcium levels. After 4-14 days, the animals are sacrificed and the BMD, total bone mass, and bone fracture strength can be measured. In addition, osteoclast numbers of certain bones can be analyzed.

In order for a compound to be evaluated as successful, the compound should be equivalent to or preferably better than the control protein (OPG-Fc or RANK-Fc) in increasing bone fracture strength, and the osteoclast numbers should preferably be lower or at least not higher than in animals treated with control protein. In addition, it is desirable that the half-life of the protein of interest is increased, preferably by at least 50% compared to that of the control protein.

1. A polypeptide having an amino acid sequence that differs from and is at least about 70% identical to the amino acid sequence of hRANK, and which has a binding affinity to RANKL that is at least as high as the binding affinity of hRANK to RANKL, as determined by the functional competition assay described herein.

2. The polypeptide of claim 1, which has an increased binding affinity to RANKL compared to the binding affinity of hRANK in the functional competition assay.

3. The polypeptide of claim 1 or 2, having an amino acid sequence that is at least about 75% identical to the amino acid sequence of hRANK, e.g. at least about 80%, 85%, 90% or 95%.

4. The polypeptide of any of claims 1-3, having at least one non-polypeptide moiety bound to an attachment group of the polypeptide.

5. The polypeptide of claim 4, wherein the non-polypeptide moiety is selected from the group consisting of polymer molecules, oligosaccharide moieties, lipophilic compounds and organic derivatizing agents.

6. The polypeptide of claim 5, wherein the non-polypeptide moiety is a PEG molecule.

7. The polypeptide of any of claims 1-6, which has an increased functional in vivo half-life and/or serum half-life compared to hRANK.

8. A polypeptide having an amino acid sequence that differs from and is least about 70% identical to the amino acid sequence of hOPG, and which has a binding affinity to RANKL that is at least as high as the binding affinity of hOPG to RANKL, as determined by the functional competition assay described herein.

9. The polypeptide of claim 8, which has an increased binding affinity to RANKL compared to the binding affinity of hOPG in the functional competition assay.

10. The polypeptide of claim 8 or 9, having an amino acid sequence that is at least about 75% identical to the amino acid sequence of hOPG, e.g. at least about 80%, 85%, 90% or 95%.

11. The polypeptide of any of claims 8-10, having at least one non-polypeptide moiety bound to an attachment group of the polypeptide.

12. The polypeptide of claim 11, wherein the non-polypeptide moiety is selected from the group consisting of polymer molecules, oligosaccharide moieties, lipophilic compounds and organic derivatizing agents.

13. The polypeptide of claim 12, wherein the non-polypeptide moiety is a PEG molecule.

14. The polypeptide of any of claims 8-13, which has an increased functional in vivo half-life and/or serum half-life compared to hOPG.

15. A polypeptide having an amino acid sequence that is at least 40% identical to the amino acid sequence of hRANK and at least 40% identical to the amino acid sequence of hOPG, and which has a binding affinity to RANKL at least as high as the binding affinity of hRANK and hOPG to RANKL, as determined by the functional competition assay described herein.

16. The polypeptide of claim 15, which has an increased binding affinity to RANKL compared to the binding affinity of hRANK and hOPG in the functional competition assay.

17. The polypeptide of claim 15 or 16, having an amino acid sequence that is at least about 45% identical to the amino acid sequence of hRANK and/or hOPG, e.g. at least about 50%, 55%, 60%, 65%, 70%, 75% or 80%.

18. The polypeptide of any of claims 15-17, having at least one non-polypeptide moiety bound to an attachment group of the polypeptide.

19. The polypeptide of claim 18, wherein the non-polypeptide moiety is selected from the group consisting of polymer molecules, oligosaccharide moieties, lipophilic compounds and organic derivatizing agents.

20. The polypeptide of claim 19, wherein the non-polypeptide moiety is a PEG molecule.

21. A chimeric polypeptide comprising a RANK backbone wherein at least one amino acid residue of the RANK backbone has been substituted with the corresponding amino acid residue from an OPG polypeptide as determined by a sequence alignment.

22. The chimeric polypeptide of claim 21, wherein at least 2, preferably at least 3, e.g. at least 4, 5, 6, 7, 8, 9 or 10, such as up to about 15 or 20 amino acid residues of the RANK backbone have been substituted with the corresponding amino acid residues from the OPG polypeptide.

23. The chimeric polypeptide of claim 21 or 22, wherein at least one amino acid residue substitution is in the TNF receptor-like domain, preferably in a ligand binding domain.

24. The chimeric polypeptide of any of claims 21-23, wherein the RANK backbone is hRANK.

25. The chimeric polypeptide of any of claims 21-24, which has an improved binding affinity to RANKL compared to the binding affinity of hRANK to RANKL, as determined by the functional competition assay described herein.

26. The chimeric polypeptide of any of claims 21-25, having at least one non-polypeptide moiety bound to an attachment group of the polypeptide.

27. A chimeric polypeptide comprising an OPG backbone wherein at least one amino acid residue of the OPG backbone has been substituted with the corresponding amino acid residue from a RANK polypeptide as determined by a sequence alignment.

28. The chimeric polypeptide of claim 27, wherein at least 2, preferably at least 3, e.g. at least 4, 5, 6, 7, 8, 9 or 10, such as up to about 15 or 20 amino acid residues of the OPG backbone have been substituted with the corresponding amino acid residues from the RANK polypeptide.

29. The chimeric polypeptide of claim 27 or 28, wherein at least one amino acid residue substitution is in the TNFR-like domain, preferably in a ligand binding domain.

30. The chimeric polypeptide of any of claims 27-29, wherein the OPG backbone is hOPG.

31. The chimeric polypeptide of any of claims 27-30, which has an improved binding affinity to RANKL compared to the binding affinity of hOPG to RANKL, as determined by the functional competition assay described herein.

32. The chimeric polypeptide of any of claims 27-31, having at least one non-polypeptide moiety bound to an attachment group of the polypeptide.

33. A method for obtaining a nucleic acid encoding a recombinant polypeptide having RANKL binding activity, the method comprising:
(a) creating a library of recombinant polynucleotides encoding one or more recombinant RANK polypeptides; and
(b) screening the library to identify a recombinant polynucleotide encoding a recombinant polypeptide with a binding affinity to RANKL at least as high as the binding affinity of hRANK to RANKL.

34. The method of claim 34, comprising selecting at least one recombinant polynucleotide encoding a recombinant polypeptide with a binding affinity to RANKL higher than the binding affinity of hRANK to RANKL.

35. The method of claim 34, wherein said library is created by subjecting a plurality of parental polynucleotides to site-directed or random mutagenesis to produce at least one recombinant RANK polynucleotide encoding said improved recombinant polypeptide

36. The method of claim 33, wherein said library is created by shuffling a plurality of parental polynucleotides to produce at least one recombinant RANK polynucleotide encoding said improved recombinant polypeptide.

37. The method of claim 36, wherein said parental polynucleotides are homologous.

38. The method of claim 36, wherein said parental polynucleotides are shuffled in a plurality of cells selected from prokaryotes and eukaryotes, e.g. in eukaryotic cells selected from bacteria, yeast, fungi and mammalian cells.

39. The method of claim 36, further comprising:
(c) recombining at least one distinct or improved recombinant polynucleotide with a further polynucleotide encoding a polypeptide with RANKL binding affinity, which further polynucleotide is identical to or different from one or more of said plurality of parental polynucleotides, to produce a library of recombinant polynucleotides;
(d) screening said library to identify at least one further distinct or improved recombinant polynucleotide encoding a RANKL binding polypeptide that exhibits a further improvement or distinct property compared to a polypeptide encoded by said plurality of parental polynucleotides; and, optionally,
(e) repeating (c) and (d) until said resulting further distinct or improved recombinant polynucleotide shows an additionally distinct or improved property.

40. The method of claim 36, wherein said recombinant polynucleotides are present in one or more cells selected from bacterial, yeast, fungal and mammalian cells, and said method comprises:
pooling multiple separate polynucleotides;
screening said resulting pooled polynucleotides to identify an improved recombinant polynucleotide encoding a polypeptide that exhibits an improved binding affinity to RANKL compared to a polypeptide encoded by a non-recombinant activity polynucleotide; and
cloning said improved recombinant nucleic acid.

41. The method of claim 40, further comprising transducing said improved polynucleotide into a member selected from a prokaryote and a eukaryote.

42. The method of claim 36, wherein said shuffling of a plurality of parental polynucleotides comprises at least one shuffling technique selected from family gene shuffling, individual gene shuffling and in silico shuffling.

43. A library of recombinant polynucleotides encoding at least one polypeptide with binding affinity to RANKL, wherein said library is made by the method of any of claims 33-42.

44. The library of claim 43, wherein polypeptides encoded by said recombinant polynucleotides are displayed on the surface of phage, bacteria cells, yeast cells or mammalian cells.

45. A nucleic acid encoding a polypeptide with binding affinity to RANKL, wherein said nucleic acid is prepared by the method of any of claims 33-42.

46. A nucleic acid shuffling mixture, comprising: at least three homologous DNAs, each of which is derived from a polynucleotide encoding a polypeptide selected from a parent RANK polypeptide, a polypeptide fragment having RANKL binding affinity, and combinations thereof.

47. The nucleic acid shuffling mixture of claim 46, wherein said at least three homologous DNAs are present in cell culture or in vitro.

48. A polypeptide having RANKL binding affinity encoded by a nucleic acid produced by the method of any of claims 33-42.

49. A method for obtaining a nucleic acid encoding a recombinant polypeptide having RANKL binding activity, the method comprising:
(a) creating a library of recombinant polynucleotides encoding one or more recombinant OPG polypeptides; and
(b) screening the library to identify a recombinant polynucleotide encoding a recombinant polypeptide with a binding affinity to RANKL at least as high as the binding affinity of hOPG to RANKL.

50. The method of claim 49, comprising selecting at least one recombinant polynucleotide encoding a recombinant polypeptide with a binding affinity to RANKL higher than the binding affinity of hOPG to RANKL.

51. The method of claim 49, wherein said library is created by subjecting a plurality of parental polynucleotides to site-directed or random mutagenesis to produce at least one recombinant OPG polynucleotide encoding said improved recombinant polypeptide

52. The method of claim 49, wherein said library is created by shuffling a plurality of parental polynucleotides to produce at least one recombinant OPG polynucleotide encoding said improved recombinant polypeptide.

53. The method of claim 52, wherein said parental polynucleotides are homologous.

54. The method of claim 52, wherein said parental polynucleotides are shuffled in a plurality of cells selected from prokaryotes and eukaryotes, e.g. in eukaryotic cells selected from bacteria, yeast, fungi and mammalian cells.

55. The method of claim 52, further comprising:
(c) recombining at least one distinct or improved recombinant polynucleotide with a further polynucleotide encoding a polypeptide with RANKL binding affinity, which further polynucleotide is identical to or different from one or more of said plurality of parental polynucleotides, to produce a library of recombinant polynucleotides;
(d) screening said library to identify at least one further distinct or improved recombinant polynucleotide encoding a RANKL binding polypeptide that exhibits a further improvement or distinct property compared to a polypeptide encoded by said plurality of parental polynucleotides; and, optionally,
(e) repeating (c) and (d) until said resulting further distinct or improved recombinant polynucleotide shows an additionally distinct or improved property.

56. The method of claim 52, wherein said recombinant polynucleotides are present in one or more cells selected from bacterial, yeast, fungal and mammalian cells, and said method comprises:
pooling multiple separate polynucleotides;
screening said resulting pooled polynucleotides to identify an improved recombinant polynucleotide encoding a polypeptide that exhibits an improved binding affinity to RANKL compared to a polypeptide encoded by a non-recombinant activity polynucleotide; and
cloning said improved recombinant nucleic acid.

57. The method of claim 56, further comprising transducing said improved polynucleotide into a member selected from a prokaryote and a eukaryote.

58. The method of claim 52, wherein said shuffling of a plurality of parental polynucleotides comprises at least one shuffling technique selected from family gene shuffling, individual gene shuffling and in silico shuffling.

59. A library of recombinant polynucleotides encoding at least one polypeptide with binding affinity to RANKL, wherein said library is made by the method of any of claims 49-58.

60. The library of claim 58, wherein polypeptides encoded by said recombinant polynucleotides are displayed on the surface of phage, bacteria cells, yeast cells or mammalian cells.

61. A nucleic acid encoding a polypeptide with binding affinity to RANKL, wherein said nucleic acid is prepared by the method of any of claims 49-58.

62. A nucleic acid shuffling mixture, comprising: at least three homologous DNAs, each of which is derived from a polynucleotide encoding a polypeptide selected from a parent OPG polypeptide, a polypeptide fragment having RANKL binding affinity, and combinations thereof.

63. The nucleic acid shuffling mixture of claim 62, wherein said at least three homologous DNAs are present in cell culture or in vitro.

64. A polypeptide having RANKL binding affinity encoded by a nucleic acid produced by the method of any of claims 49-58.

65. A polypeptide conjugate exhibiting RANKL-binding activity, comprising a RANK polypeptide that differs from wild-type human RANK in that at least one amino acid residue acid residue comprising an attachment group for a non-polypeptide moiety has been introduced or removed, and having at least one non-polypeptide moiety bound to an attachment group of the polypeptide.

66. The polypeptide conjugate of claim 65, wherein the RANK polypeptide is a RANK variant as defined in any of claims 1-7 or 21-26 or encoded by a nucleic acid produced by the method of any of claims 33-42.

67. A polypeptide conjugate exhibiting RANKL-binding activity, comprising an OPG polypeptide that differs from wild-type human OPG in that at least one amino acid residue acid residue comprising an attachment group for a non-polypeptide moiety has been introduced or removed, and having at least one non-polypeptide moiety bound to an attachment group of the polypeptide.

68. The polypeptide conjugate of claim 67, wherein the OPG polypeptide is an OPG variant as defined in any of claims 8-14 or 27-32 or encoded by a nucleic acid produced by the method of any of claims 49-58.

69. An oligomeric fusion protein comprising at least two RANK monomers, at least two OPG monomers, or at least one RANK monomer and at least one OPG monomer, wherein at least one monomer of the fusion protein is a RANK and/or OPG variant as defined in any of claims 1-32 or encoded by a nucleic acid produced by the method of any of claims 33-42 or 49-58.

70. The fusion protein of claim 69, wherein the monomers are joined by a peptide bond or a peptide linker, or by a PEG molecule.

71. The fusion protein of claim 69, comprising at least one RANKL-binding monomeric fusion protein, wherein said monomeric fusion protein is produced as a protein fused in frame with an immunoglobulin Fc polypeptide or a GCN4 leucine zipper.

72. A composition comprising a polypeptide according to any of claims 1-31 or 65-71 or encoded by a nucleic acid produced by the method of any of claims 33-42 or 49-58, and at least one pharmaceutically acceptable carrier or excipient.

73. Use of a polypeptide according to any of claims 1-31 or 65-71 or encoded by a nucleic acid produced by the method of any of claims 33-42 or 49-58, or a composition according to claim 72, as a pharmaceutical.

74. Use of a polypeptide according to any of claims 1-31 or 65-71 or encoded by a nucleic acid produced by the method of any of claims 33-42 or 49-58, or a composition according to claim 72, for the preparation of a medicament for the prevention or treatment of osteoporosis or other bone diseases or other diseases associated with binding of RANKL to the RANK receptor.

75. A method for preventing or treating osteoporosis or other bone diseases or other diseases associated with binding of RANKL to the RANK receptor, the method comprising administering to a patient in need thereof an effective amount of a polypeptide according to any of claims 1-32 or 65-71 or encoded by a nucleic acid produced by the method of any of claims 33-42 or 49-58, or a composition according to claim 72.

76. An expression vector comprising a nucleic acid produced by the method of any of claims 33-42 or 49-58.

77. A host cell comprising an expression vector according to claim 76.

78. A method for producing a polypeptide having binding affinity to RANKL, comprising culturing a host cell according to claim 77 under conditions conducive for expression of the polypeptide, and recovering the polypeptide.

79. The method of claim 78, wherein a) the polypeptide comprises at least one N- or O-glycosylation site and the host cell is a eukaryotic host cell capable of in vivo glycosylation, and/or b) the polypeptide is subjected to conjugation to a non-polypeptide moiety in vitro.

80. The chimeric polypeptide of claim 21, comprising all or part of at least one TNF receptor-like domain of OPG as defined in FIG. 4B.

81. The chimeric polypeptide of claim 80, wherein said part comprises at least one ligand binding subsequence of OPG comprising at least three amino acid residues as defined in FIG. 4B.

82. The chimeric polypeptide of claim 27, comprising all or part of at least one TNF receptor-like domain of RANK as defined in FIG. 4B.

83. The chimeric polypeptide of claim 82, wherein said part comprises at least one ligand binding subsequence of RANK comprising at least three amino acid residues as defined in FIG. 4B.

84. A method for obtaining a nucleic acid encoding a recombinant polypeptide having a desired RANKL binding activity, the method comprising:
(a) providing a polynucleotide encoding a recombinant chimeric polypeptide comprising at least one ligand binding sequence from an OPG domain and at least one ligand binding sequence from a RANK domain;
(b) subjecting said polynucleotide to mutagenesis to create a library of recombinant polynucleotides encoding one or more recombinant chimeric polypeptides; and
(c) screening the library to identify a recombinant polynucleotide encoding a recombinant polypeptide with a desired binding affinity to RANKL.

85. The method of claim 84, wherein said recombinant chimeric polypeptide in (a) comprises at least one OPG domain and at least one RANK domain.

86. The method of claim 84 or 85, wherein mutagenesis is performed using at least one of site-directed mutagenesis, random mutagenesis and shuffling.

87. A polypeptide having an amino acid sequence that is least about 70% identical to the amino acid sequence of hOPG(22-194) and wherein one or more of the amino acid residues selected from T71, K108, R111, and T154 have been substituted with a different amino acid residue.

88. A polypeptide comprising the amino acid sequence hOPG(22-194) wherein one or more of the amino acid residues selected from T71, K108, R111, and T154 have been substituted with a different amino acid residue.

89. The polypeptide of claim 87 or 88 wherein T71 has been substituted with A.

90. The polypeptide of any one of claims 87-89 wherein K108 has been substituted with N.

91. The polypeptide of any one of claims 87-90 wherein R111 has been substituted with W.

92. The polypeptide of any one of claims 87-91 wherein T154 has been substituted with L.

93. The polypeptide of any one of claims 87-92 whereint the polypeptide is selected from the group comprising T71A,K108N-hOPG(22-194), R111W-hOPG(22-194), K108M,R111W-hOPG(22-194), and T154L-hOPG(22-194).
